(19) United States
(12) Patent Application Publication
(10) Pub. No.: US 2025/0372262 A1
(43) Pub. Date: Dec. 4, 2025

(54) METHODS AND SYSTEMS FOR DETECTING SKIN CONDITIONS

(71) Applicant: DERMTECH, LLC, San Diego, CA (US)

(72) Inventors: Ryan CEDENO, Escondido, CA (US); Zuxu YAO, San Diego, CA (US); Loren CLARKE, San Diego, CA (US); Burkhard JANSEN, San Diego, CA (US); James ROCK, San Diego, CA (US); John DOBAK, III, La Jolla, CA (US)

(21) Appl. No.: 19/304,519

(22) Filed: Aug. 19, 2025

Related U.S. Application Data
(63) Continuation-in-part of application No. PCT/US2025/022128, filed on Mar. 28, 2025.
Application No. PCT/US2025/022128 is a continuation-in-part of application No. 18/056,157, filed on Nov. 16, 2022.
Application No. 18/056,157 is a division of application No. 16/828,289, filed on Mar. 24, 2020, now Pat. No. 11,578,373.
(63) Continuation-in-part of application No. 17/534,894, filed on Nov. 24, 2021, now Pat. No. 12,029,075.
Application No. 17/534,894 is a continuation of application No. 16/603,435, filed on Oct. 7, 2019.
(63) Continuation-in-part of application No. 16/874,473, filed on May 14, 2020, now abandoned.
Application No. 16/874,473 is a continuation of application No. PCT/US2019/031203, filed on May 7, 2019.
(63) Continuation-in-part of application No. 29/796,477, filed on Jun. 24, 2021, now Pat. No. D1097166.
Application No. 29/796,477 is a continuation of application No. 17/183,589, filed on Feb. 24, 2021.
Application No. 17/183,589 is a continuation of application No. 17/002,676, filed on Aug. 25, 2020.
Application No. 17/002,676 is a continuation of application No. 16/886,611, filed on May 28, 2020, now abandoned.
Application No. 16/886,611 is a continuation of application No. 15/571,247, filed on Nov. 1, 2017, now Pat. No. 10,709,428.
(63) Continuation-in-part of application No. 17/183,589, filed on Feb. 24, 2021.
(63) Continuation-in-part of application No. 17/002,676, filed on Aug. 25, 2020.
(63) Continuation-in-part of application No. 17/217,568, filed on Mar. 30, 2021.
Application No. 17/217,568 is a continuation of application No. 17/195,541, filed on Mar. 8, 2021, now Pat. No. 11,753,687.
Application No. 17/195,541 is a continuation of application No. 16/522,291, filed on Jul. 25, 2019, now Pat. No. 11,332,795.
Application No. 16/522,291 is a continuation of application No. 14/832,966, filed on Aug. 21, 2015, now Pat. No. 10,407,729.
Application No. 14/832,966 is a continuation of application No. 14/172,784, filed on Feb. 4, 2014, now abandoned.
(63) Continuation-in-part of application No. 17/315,199, filed on May 7, 2021.
(63) Continuation-in-part of application No. 17/534,177, filed on Nov. 23, 2021.
(63) Continuation-in-part of application No. 17/832,394, filed on Jun. 3, 2022.
(63) Continuation-in-part of application No. 18/548,321, filed on Aug. 29, 2023.
(63) Continuation-in-part of application No. 18/555,195, filed on Oct. 12, 2023.
(63) Continuation-in-part of application No. 18/602,952, filed on Mar. 12, 2024.
Application No. 18/602,952 is a continuation of application No. 16/969,526, filed on Aug. 12, 2020, now Pat. No. 11,976,332.
(63) Continuation-in-part of application No. 18/865,624, filed on Jan. 1, 0001.
(63) Continuation-in-part of application No. PCT/US2024/038611, filed on Jul. 18, 2024.
(63) Continuation-in-part of application No. 18/056,157, filed on Nov. 16, 2022.
(60) Provisional application No. 62/156,091, filed on May 1, 2015.
(60) Provisional application No. 62/630,627, filed on Feb. 14, 2018.
(60) Provisional application No. 62/669,297, filed on May 9, 2018.
(60) Provisional application No. 62/824,163, filed on Mar. 26, 2019.
(60) Provisional application No. 63/022,364, filed on May 8, 2020.
(60) Provisional application No. 63/117,946, filed on Nov. 24, 2020.
(60) Provisional application No. 63/155,665, filed on Mar. 2, 2021.
(60) Provisional application No. 63/174,345, filed on Apr. 13, 2021.
(60) Provisional application No. 63/175,514, filed on Apr. 15, 2021.
(60) Provisional application No. 63/197,212, filed on Jun. 4, 2021.
(60) Provisional application No. 63/245,118, filed on Sep. 16, 2021.
(60) Provisional application No. 63/285,328, filed on Dec. 2, 2021.
(60) Provisional application No. 63/322,968, filed on Mar. 23, 2022.
(60) Provisional application No. 63/341,963, filed on May 13, 2022.
(60) Provisional application No. 63/527,670, filed on Jul. 19, 2023.
(60) Provisional application No. 63/571,039, filed on Mar. 28, 2024.

(51) Int. Cl. G16H 50/20; G16B 20/00; G16B 25/10; G16B 40/20; G16H 10/40; G16H 10/60

(52) U.S. Cl. CPC G16H 50/20; G16B 20/00; G16B 25/10; G16B 40/20; G16H 10/40; G16H 10/60

(57) ABSTRACT

Disclosed herein, in certain embodiments, are systems and methods of detecting the presence of a skin condition using a machine learning model based on molecular risk factors. In some instances, the skin condition is cancer, such as cutaneous T cell lymphoma (CTCL). In some cases, the skin cancer can be mycosis fungoides (MF) or Sézary syndrome (SS).

### CROSS-REFERENCE

This application is a Continuation-in-Part of International Application No. PCT/US2025/022128, filed on Mar. 28, 2025, which claims the benefit of U.S. Provisional Application No. 63/571,039, filed on Mar. 28, 2024. International Application No. PCT/US2025/022128 is a Continuation-in-Part of U.S. application Ser. No. 18/056,157, filed on Nov. 16, 2022, which is a divisional of U.S. application Ser. No. 16/828,289, issued as U.S. Pat. No. 11,578,373, filed Mar. 24, 2020; which claims the benefit of U.S. Provisional Application No. 62/824,163, filed Mar. 26, 2019.

This application a Continuation-in-Part of International Application No. PCT/US2024/038611, filed on Jul. 18, 2024, which claims the benefit of U.S. Provisional Application No. 63/527,670, filed Jul. 19, 2023.

This application is also a Continuation-in-Part of U.S. application Ser. No. 18/056,157, filed Nov. 16, 2022, which is a divisional of U.S. application Ser. No. 16/828,289, filed Mar. 24, 2020, now U.S. Pat. No. 11,578,373, which claims the benefit of U.S. Provisional Application No. 62/824,163, filed Mar. 26, 2019.

This application is also a Continuation-in-Part of U.S. application Ser. No. 17/354,894, filed Jun. 22, 2021, which is a continuation of U.S. application Ser. No. 16/603,435, filed on Oct. 7, 2019, which is a National Stage Entry of International Application No. PCT/US2018/026902, filed Apr. 10, 2018, which claims the benefit of U.S. Provisional Application No. 62/483,834, filed Apr. 10, 2017 and U.S. Provisional Application No. 62/562,250, filed Sep. 22, 2017.

This application is also a Continuation-in-Part of U.S. application Ser. No. 16/874,473, filed May 14, 2020, which is a continuation of International Application No. PCT/US2019/031203, filed May 7, 2019, which claims the benefit of U.S. Provisional Application No. 62/669,297, filed May 9, 2018.

This application is also a Continuation-in-Part of U.S. Design application Ser. No. 29/796,477, filed Jun. 24, 2021, which is a continuation of U.S. application Ser. No. 17/183,589, filed Feb. 24, 2021, which is a continuation of U.S. application Ser. No. 17/002,676, filed Aug. 25, 2020, which is a continuation of U.S. application Ser. No. 16/886,611, filed May 28, 2020, which is a continuation of U.S. application Ser. No. 15/571,247, filed Nov. 1, 2017, now U.S. Pat. No. 10,709,428, which is a National Stage Entry of International Application No. PCT/US2016/030287, filed Apr. 29, 2016, which claims the benefit of U.S. Provisional Application No. 62/156,091, filed May 1, 2015.

This application is also a Continuation-in-Part of U.S. application Ser. No. 17/183,589, filed Feb. 24, 2021, which is a continuation of U.S. application Ser. No. 17/002,676, filed Aug. 25, 2020, which is a continuation of U.S. application Ser. No. 16/886,611, filed May 28, 2020, which is a continuation of U.S. application Ser. No. 15/571,247, filed Nov. 1, 2017, now U.S. Pat. No. 10,709,428, which is a National Stage Entry of International Application No. PCT/US2016/030287, filed Apr. 29, 2016, which claims the benefit of U.S. Provisional Application No. 62/156,091, filed May 1, 2015.

This application is also a Continuation-in-Part of U.S. application Ser. No. 17/002,676, filed Aug. 25, 2020, which is a continuation of U.S. application Ser. No. 16/886,611, filed May 28, 2020, which is a continuation of U.S. application Ser. No. 15/571,247, filed Nov. 1, 2017, now U.S. Pat. No. 10,709,428, which is a National Stage Entry of International Application No. PCT/US2016/030287, filed Apr. 29, 2016, which claims the benefit of U.S. Provisional Application No. 62/156,091, filed May 1, 2015.

This application is also a Continuation-in-Part of U.S. application Ser. No. 17/217,568, filed Mar. 30, 2021, which is a continuation of U.S. application Ser. No. 17/195,541, filed Mar. 8, 2021, now U.S. Pat. No. 11,753,687, which is a continuation of U.S. application Ser. No. 16/522,291, filed Jul. 25, 2019, now U.S. Pat. No. 11,332,795, which is a continuation of U.S. application Ser. No. 14/832,966, filed Aug. 21, 2015, now U.S. Pat. No. 10,407,729, which is a continuation of U.S. application Ser. No. 14/172,784, filed Feb. 4, 2014.

This application is also a Continuation-in-Part of U.S. application Ser. No. 17/315,199, filed May 7, 2021, which claims the benefit of U.S. Provisional Application No. 63/022,364, filed May 8, 2020.

This application is also a Continuation-in-Part of U.S. application Ser. No. 17/534,177, filed Nov. 23, 2021, which claims the benefit of U.S. Provisional Application No. 63/117,946, filed Nov. 24, 2020.

This application is also a Continuation-in-Part of U.S. application Ser. No. 17/832,394, filed Jun. 3, 2022, which claims the benefit of U.S. Provisional Application No. 63/197,212, filed Jun. 4, 2021, U.S. Provisional Application No. 63/285,328, filed Dec. 2, 2021, and U.S. Provisional Application No. 63/322,968, filed Mar. 23, 2022.

This application is also a Continuation-in-Part of U.S. application Ser. No. 18/548,321, filed Aug. 29, 2023, which is a National Stage Entry of International Application No. PCT/US2022/018274, filed Mar. 1, 2022, which claims the benefit of U.S. Provisional Application No. 63/155,665, filed Mar. 2, 2021.

This application is also a Continuation-in-Part of U.S. application Ser. No. 18/555,195, filed Oct. 12, 2023, which is a National Stage Entry of International Application No. PCT/US2022/024488, filed Apr. 12, 2022, which claims the benefit of U.S. Provisional Application No. 63/174,345, filed Apr. 13, 2021, U.S. Provisional Application No. 63/175,514, filed Apr. 15, 2021 and U.S. Provisional Application No. 63/245,118, filed Sep. 16, 2021.

This application is also a Continuation-in-Part of U.S. application Ser. No. 18/602,952, filed Mar. 12, 2024, which is a continuation of U.S. application Ser. No. 16/969,526, filed Aug. 12, 2020, now U.S. Pat. No. 11,976,332, which is a National Stage Entry of International Application No. PCT/US2019/018102, filed Feb. 14, 2019, which claims the benefit of U.S. Provisional Application No. 62/630,627, filed Feb. 14, 2018.

This application is also a Continuation-in-Part of U.S. application Ser. No. 18/865,624, filed Nov. 13, 2024, which is a National Stage Entry of International Application No. PCT/US2023/066973, filed May 12, 2023, which claims the benefit of U.S. Provisional Application No. 63/341,963, filed May 13, 2022.

Each of the above-referenced patent applications is incorporated by reference in its entirety herein.

### BACKGROUND OF THE DISCLOSURE

Skin diseases are some of the most common human illnesses and represent an important global burden in healthcare. Three skin diseases are in the top ten most prevalent diseases worldwide, and eight fall into the top 50. When considered collectively, skin conditions range from being the second to the 11th leading causes of years lived with disability.

Non-melanoma skin cancer (NMSC) is the most common type of skin cancer and encompasses a collection of skin cancers including angiosarcoma, basal cell carcinoma (BCC), cutaneous B-cell lymphoma, cutaneous T-cell lymphoma (CTCL), dermatofibrosarcoma protuberans, Merkel cell carcinoma, sebaceous carcinoma, and squamous cell carcinoma of the skin (SCC). Cutaneous T-cell lymphoma (CTCL) is a class of non-Hodgkin lymphoma due to altered T cells. In general, the annual incidence of CTCL is about 0.5 per 100,000 in the population and can be observed in adults with a median age of 55-60 years. Further, there are about 7 clinical stages for CTCL (IA, IB, IIA, IIB, III, IVA, and IVB).

CTCL further comprises several subtypes including, but not limited to, mycosis fungoides (MF), Sézary syndrome (SS), pagetoid reticulosis, granulomatous slack skin, lymphomatoid papulosis, pityriasis lichenoides chronica, pityriasis lichenoides et varioliformis acuta, CD30+ cutaneous T-cell lymphoma, secondary cutaneous CD30+ large cell lymphoma, non-mycosis fungoides CD30− cutaneous large T-cell lymphoma, pleomorphic T-cell lymphoma, Lennert lymphoma, subcutaneous T-cell lymphoma, angiocentric lymphoma, and blastic NK-cell lymphoma. Mycosis fungoides (MF) is the most common type of CTCL and the disease phenotype can vary among patients. Sézary syndrome (SS) is an advanced and aggressive subtype of CTCL and is characterized by the presence of malignant lymphoma cells in the blood.

Heterogeneity is observed in the molecular changes (or dysregulated gene expression) between CTCL patients and in some instances within the same patient overtime. In some cases, this heterogeneity is attributed to the different causes which convert normal T cells into malignant T cells. In additional cases, this heterogeneity contributes to the difficulties in detecting the presence of CTCL and in diagnosing a subject in having CTCL. Subjects having CTCL may present with one or more symptoms that indicate other skin diseases, disorders, or conditions. For example, CTCL can present as patches, plaques, tumors, and/or generalized erythroderma, and such symptoms can be temporarily resolved with topical and/or over-the-counter treatments, such as topical corticosteroids. Thus, CTCL can be misdiagnosed as a benign skin disorder, such as eczema, psoriasis, atopic dermatitis, and/or contact dermatitis.

U.S. Pat. No. 11,578,373 describes detection of skin cancer based on molecular risk factors. However, such disclosures do not distinguish among skin cancer and non-cancerous conditions based on a gene expression profile.

### SUMMARY OF THE DISCLOSURE

Disclosed herein, in certain embodiments, is a method of detecting the presence of a skin cancer based on molecular risk factors. In some instances, the skin cancer is a non-Hodgkin lymphoma. In some instances, the skin cancer is cutaneous T cell lymphoma (CTCL). In some instances, the non-Hodgkin lymphoma is CTCL. In some cases, the skin cancer is mycosis fungoides (MF) or Sézary syndrome (SS). In some cases, the method described herein can distinguish between skin cancer (e.g., CTCL), other skin disorders, diseases, and/or conditions (e.g., atopic dermatitis, lupus, rubeola, acne, hemangioma, psoriasis, eczema, candidiasis, impetigo, shingles, leprosy, Crohn's disease, inflammatory dermatoses, bullous disease, solar lentigo, dermatofibrosarcoma protuberans, dysplastic nevi), and normal (e.g., healthy or non-diseased) skin. In some embodiments, the method described herein includes non-transitory computer readable media storing computer-executable instructions that generate a diagnosis of CTCL and/or other skin disorder, disease or condition, based on gene expression data from a sample.

In some implementations, a non-transitory computer readable media storing computer-executable instructions that, when executed by at least one processor, cause a computing device to receive gene data, the gene data extracted from a tissue sample collected using an adhesive skin sample collector, input the gene data into one or more diagnostic models, generate prediction data using the one or more diagnostic models, the prediction data indicating if the tissue sample includes a skin disease based on the gene data, and generate output data indicating if the tissue sample includes the skin disease.

In some implementations, a method for detecting a skin condition, the method comprising receiving gene data, the gene data extracted from a tissue sample collected using an adhesive patch, inputting the gene data into one or more diagnostic models, generating prediction data using the one or more diagnostic models, the prediction data indicating if the tissue sample includes the skin condition based on the gene data, and generating output data indicating if the tissue sample includes the skin condition.

In some implementations, a system for detecting a skin disease, the system comprising: a skin diagnostic system in communication with a computing device and one or more databases over a network, the skin diagnostic system receiving gene data, the gene data extracted from a tissue sample collected by a non-invasive skin sample collector, one or more diagnostic models processing the gene data to generate prediction data associated with the skin disease, and an output generation system generating output data indicating if the tissue sample includes the skin disease.

Disclosed herein, in certain embodiments, is a method of detecting gene expression level of FYN binding protein (FYB), IL2 inducible T-cell kinase (ITK), interleukin 26 (IL26), signal transducer and activator of transcription 5A (STAT5A), TRAF3 interacting protein 3 (TRAF3IP3), granulysin (GNLY), dynamin 3 (DNM3), tumor necrosis factor superfamily member 11 (TNFSF11), C-C chemokine ligand 27 (CCL27), C-X-C chemokine ligand 8 (CXCL8), C-X-C chemokine ligand 9 (CXCL9), C-X-C chemokine ligand 10 (CXCL10), tumor necrosis factor (TNF) or a combination thereof in a subject in need thereof, comprising: (a) isolating nucleic acids from a skin sample obtained from the subject, wherein the skin sample comprises cells from the stratum corneum; and (b) detecting the expression levels of FYB, LEF1, GNLY, DMN3, ITK, IL26, STAT5, TRAF3IP3, TNFSF11, CCL27, CXCL8, CXCL9, CXCL10, TNF, or a combination thereof, by contacting the isolated nucleic acids with a set of probes that recognizes FYB, LEF1, GNLY, DMN3, ITK, IL26, STAT5, TRAF3IP3, TNFSF11, CCL27, CXCL8, CXCL9, CXCL10, TNF, or a combination thereof, and detects binding between FYB, LEF1, GNLY, DMN3, ITK, IL26, STAT5, TRAF3IP3, TNFSF11, CCL27, CXCL8, CXCL9, CXCL10, TNF, or a combination thereof and the set of probes. In some embodiments, the method comprises detecting the expression levels of ITK, STAT5A, and TNFSF11. In some embodiments, the method comprises detecting the expression levels of ITK, IL26, STAT5A, and TNFSF11. In some embodiments, the method comprises detecting the expression levels of FYB, ITK, IL26, STAT5A, and TNFSF11. In some embodiments, the method comprises detecting the expression levels of FYB, ITK, IL26, STAT5A, TRAF3IP3, and TNFSF11. In some embodiments, the method comprises detecting the expression levels of FYB, ITK, IL26, STAT5A, TRAF3IP3, DNM3, and TNFSF11. In some embodiments, the method comprises detecting the expression levels of FYB, ITK, IL26, STAT5A, TRAF3IP3, GNLY, DNM3, and TNFSF11. In some embodiments, the expression level is an elevated gene expression level, compared to a gene expression level of an equivalent gene from a control sample. In some embodiments, the gene expression level of FYB, ITK, IL26, STAT5A, TRAF3IP3, DNM3, TNFSF11, or a combination thereof is elevated. In some embodiments, the expression level is a down-regulated gene expression level, compared to a gene expression level of an equivalent gene from a control sample. In some embodiments, the gene expression level of GNLY is down-regulated. In some embodiments, the set of probes recognizes at least one but no more than eight genes. In some embodiments, the method further comprises detecting the expression levels of TOX, LEF1, CCR4, POU2AF1, GTSF1, PLS3, MMP12, LCK, NEDD4L, or a combination thereof. In some embodiments, the detecting comprises contacting the isolated nucleic acids with an additional set of probes that recognizes TOX, LEF1, CCR4, POU2AF1, GTSF1, PLS3, MMP12, LCK, NEDD4L, or a combination thereof, and detects binding between TOX, LEF1, CCR4, POU2AF1, GTSF1, PLS3, MMP12, LCK, NEDD4L, or a combination thereof and the additional set of probes. In some embodiments, the expression level(s) of one or more additional genes, such as those disclosed in Table 1, can be detected. In some embodiments, the additional set of probes recognizes one but no more than nine genes. In some embodiments, the cells from the stratum corneum comprise T cells or components of T cells. In some embodiments, the cells from the stratum corneum comprise keratinocytes. In some embodiments, the skin sample does not comprise melanocytes. In some embodiments, the skin sample is obtained by applying an adhesive patch to a skin region of the subject in a manner sufficient to adhere cells to the adhesive patch, and removing the adhesive patch from the skin region in a manner sufficient to retain the adhered cells to the adhesive patch. In some embodiments, the skin sample is obtained by applying a plurality of adhesive patches to a skin region of the subject in a manner sufficient to adhere cells to each of the adhesive patches, and removing each of the adhesive patches from the skin region in a manner sufficient to retain the adhered cells to each of the adhesive patches. In some embodiments, the plurality of adhesive patches comprises at least 4 adhesive patches. In some embodiments, the skin region is a skin lesion region. In some embodiments, the subject is suspected of having cutaneous T cell lymphoma (CTCL). In some embodiments, the subject is suspected of having mycosis fungoides (MF). In some embodiments, the subject is suspected of having Sézary syndrome (SS). In some embodiments, the subject is a human.

Disclosed herein, in certain embodiments, is a method of detecting gene expression levels from a first gene classifier and a second gene classifier in a subject in need thereof, comprising: (a) isolating nucleic acids from a skin sample obtained from the subject, wherein the skin sample comprises cells from the stratum corneum; (b) detecting the expression levels of one or more genes from the first gene classifier: FYB, LEF1, GNLY, DMN3, ITK, IL26, STAT5, TRAF3IP3, TNFSF11, CCL27, CXCL8, CXCL9, CXCL10, TNF, by contacting the isolated nucleic acids with a set of probes that recognizes one or more genes from the first gene classifier, and detects binding between one or more genes from the first gene classifier and the set of probes; and (c) detecting the expression levels of one or more genes from the second gene classifier: TOX, LEF1, CCR4, POU2AF1, GTSF1, PLS3, MMP12, LCK, and NEDD4L, by contacting the isolated nucleic acids with an additional set of probes that recognizes one or more genes from the second gene classifier, and detects binding between one or more genes from the second gene classifier and the additional set of probes. In some embodiments, the method comprises detecting the expression levels of ITK, STAT5A, and TNFSF11 from the first gene classifier. In some embodiments, the method comprises detecting the expression levels of ITK, IL26, STAT5A, and TNFSF11 from the first gene classifier. In some embodiments, the method comprises detecting the expression levels of FYB, ITK, IL26, STAT5A, and TNFSF11 from the first gene classifier. In some embodiments, the method comprises detecting the expression levels of FYB, ITK, IL26, STAT5A, TRAF3IP3, and TNFSF11 from the first gene classifier. In some embodiments, the method comprises detecting the expression levels of FYB, ITK, IL26, STAT5A, TRAF3IP3, DNM3, and TNFSF11 from the first gene classifier. In some embodiments, the method comprises detecting the expression levels of FYB, ITK, IL26, STAT5A, TRAF3IP3, GNLY, DNM3, and TNFSF11 from the first gene classifier. In some embodiments, the expression level is an elevated gene expression level, compared to a gene expression level of an equivalent gene from a control sample. In some embodiments, the gene expression level of FYB, LEF1, GNLY, DMN3, ITK, IL26, STAT5, TRAF3IP3, TNFSF11, CCL27, CXCL8, CXCL9, CXCL10, TNF, or a combination thereof is elevated. In some embodiments, the expression level is a down-regulated gene expression level, compared to a gene expression level of an equivalent gene from a control sample. In some embodiments, the gene expression level of GNLY is down-regulated. In some embodiments, the set of probes recognizes at least one but no more than eight genes. In some embodiments, the additional set of probes recognizes one but no more than nine genes. In some embodiments, the nucleic acids comprise RNA, DNA, or a combination thereof. In some embodiments, the RNA is mRNA. In some embodiments, the RNA is cell-free circulating RNA. In some embodiments, the cells from the stratum corneum comprise T cells or components of T cells. In some embodiments, the cells from the stratum corneum comprise keratinocytes. In some embodiments, the skin sample does not comprise melanocytes. In some embodiments, the skin sample is obtained by applying an adhesive patch to a skin region of the subject in a manner sufficient to adhere cells to the adhesive patch, and removing the adhesive patch from the skin region in a manner sufficient to retain the adhered cells to the adhesive patch. In some embodiments, the skin sample is obtained by applying a plurality of adhesive patches to a skin region of the subject in a manner sufficient to adhere cells to each of the adhesive patches, and removing each of the adhesive patches from the skin region in a manner sufficient to retain the adhered cells to each of the adhesive patches. In some embodiments, the plurality of adhesive patches comprises at least 4 adhesive patches. In some embodiments, the skin region is a skin lesion region. In some embodiments, the subject is suspected of having cutaneous T cell lymphoma (CTCL). In some embodiments, the subject is suspected of having mycosis fungoides (MF). In some embodiments, the subject is suspected of having Sézary syndrome (SS). In some embodiments, the subject is a human.

Disclosed herein, in certain embodiments, is a method of determining the presence of cutaneous T cell lymphoma (CTCL) in a skin sample, comprising: identifying a subject suspected of having CTCL; isolating nucleic acids from a skin sample obtained from the subject by applying an adhesive patch to a skin region of the subject in a manner sufficient to adhere skin sample cells to the adhesive patch, and removing the adhesive patch from the skin sample in a manner sufficient to retain the adhered skin sample cells to the adhesive patch, wherein the skin sample cells comprise cells from the stratum corneum; and detecting an expression level of at least one target gene known to be upregulated or downregulated in subjects with CTCL, by contacting the isolated nucleic acids with a set of probes that recognize the target gene, and detecting binding between the at least one target gene and the set of probes. In some embodiments, the nucleic acids comprise mRNA. In some embodiments, the cells from the stratum corneum comprise T cells or components of T cells. In some embodiments, the cells from the stratum corneum comprise keratinocytes. In some embodiments, the skin sample does not comprise melanocytes. In some embodiments, the skin sample is obtained by applying a plurality of adhesive patches to the skin region of the subject in a manner sufficient to adhere skin sample cells to each of the adhesive patches, and removing each of the plurality of adhesive patches from the skin region in a manner sufficient to retain the adhered skin sample cells to each of the adhesive patches. In some embodiments, the skin region comprises a skin lesion. Some embodiments include determining whether the subject has CTCL based on the expression level of the at least one target gene. Some embodiments include administering a CTCL treatment to the subject based on the determination of whether the subject has CTCL. In some embodiments, the CTCL treatment comprises a steroid, interferon, chemotherapy, phototherapy, radiation therapy, or a bone marrow transplant. In some embodiments, the subject has CTCL. In some embodiments, the CTCL comprises mycosis fungoides. In some embodiments, the CTCL comprises Sézary syndrome. In some embodiments, the subject is a human. In some embodiments, the expression level is upregulated compared to a gene expression level of an equivalent gene from a control sample. In some embodiments, the expression level is downregulated compared to a gene expression level of an equivalent gene from a control sample. In some embodiments, the at least one target gene comprises a gene encoding an adapter protein, a gene encoding a tyrosine kinase, a gene encoding an interleukin, a gene encoding a transcription factor, a gene encoding a TNF receptor associated factor protein, a gene encoding a TNF, a gene encoding a TNF superfamily member, a gene encoding a saposin-like protein, a gene encoding a GTP-binding protein, a gene encoding a chromatin associated protein, a gene encoding a G-protein-coupled receptor, a gene encoding a transcriptional coactivator, a gene encoding a spermatogenesis protein, a gene encoding an actin-binding protein, a gene encoding a matrix metalloproteinase, a gene encoding a FYN-binding protein family member, a gene encoding a TEC kinase family member, a gene encoding a STAT, a gene encoding a TRAF3 interacting protein, a gene encoding a dynamin family member, a gene encoding a ubiquitin ligase, a gene encoding a thymocyte selection associated high mobility group box family member, a gene encoding a lymphoid enhancer binding factor family member, a gene encoding a C-C chemokine receptor type family member, a gene encoding an Oct binding factor family member, a gene encoding an gametocyte-specific family member, a gene encoding a plastin family member, a gene encoding a lymphocyte-specific protein tyrosine kinase family member, a gene encoding a member of the NEDD4 family of E3 HECT domain ubiquitin ligases, a gene encoding a C-C motif chemokine ligand family member, a gene encoding a chemokine, or a gene encoding a CXC chemokine, or a combination thereof. In some embodiments, the at least one target gene comprises a gene encoding a saposin-like protein, a gene encoding a FYN-binding protein family member, a gene encoding a TEC kinase family member, a gene encoding a STAT, a gene encoding a TRAF3 interacting protein, a gene encoding a CXC chemokine family member, or a combination thereof. In some embodiments, the at least one target gene comprises a gene encoding modulator of cell death, a gene encoding an antimicrobial, a gene encoding a cytokine, or a gene encoding a DNA-binding protein, or a combination thereof. In some embodiments, the at least one target gene comprises FYB, GNLY, ITK, STAT5, TRAF3IP3, CXCL10, CXCL8, and/or TNF, or a combination thereof. In some embodiments, the at least one target gene comprises a gene encoding a microRNA. In some embodiments, the microRNA comprises miR-21, miR-29b, miR-155, miR-186, miR-214, or miR-221. Some embodiments further comprise detecting the presence at least one genotype of one more additional target genes known to be mutated in subjects with CTCL, in the nucleic acids or in a separate set of nucleic acids isolated from the skin sample. In some embodiments, the nucleic acids or the separate set of nucleic acids comprise DNA. In some embodiments, determining whether the subject has CTCL further comprises determining whether the subject has CTCL based on the presence of the at least one genotype. In some embodiments, the one or more additional target genes comprise TP53, ZEB1, ARID1A, DNMT3A, CDKN2A, FAS, STAT5B, PRKCQ, RHOA, DNMT3A, PLCG1, or NFKB2.

Disclosed herein, in certain embodiments, is a method of determining the presence of a non-cancerous skin condition in a skin sample, comprising: identifying a subject suspected of having non-cancerous skin condition; isolating nucleic acids from a skin sample obtained from the subject by applying an adhesive patch to a skin region of the subject in a manner sufficient to adhere skin sample cells to the adhesive patch, and removing the adhesive patch from the skin sample in a manner sufficient to retain the adhered skin sample cells to the adhesive patch, wherein the skin sample cells comprise cells from the stratum corneum; and detecting an expression level of at least one target gene known to be upregulated or downregulated in subjects with non-cancerous skin condition, by contacting the isolated nucleic acids with a set of probes that recognize the target gene, and detecting binding between the at least one target gene and the set of probes. In some embodiments, the nucleic acids comprise mRNA. In some embodiments, the cells from the stratum corneum comprise T cells or components of T cells. In some embodiments, the cells from the stratum corneum comprise keratinocytes. In some embodiments, the skin sample does not comprise melanocytes. In some embodiments, the skin sample is obtained by applying a plurality of adhesive patches to the skin region of the subject in a manner sufficient to adhere skin sample cells to each of the adhesive patches, and removing each of the plurality of adhesive patches from the skin region in a manner sufficient to retain the adhered skin sample cells to each of the adhesive patches. In some embodiments, the skin region comprises a skin lesion. Some embodiments include determining whether the subject has non-cancerous skin condition based on the expression level of the at least one target gene. Some embodiments include administering a non-cancerous skin condition treatment to the subject based on the determination of whether the subject has non-cancerous skin condition. In some embodiments, the non-cancerous skin condition treatment comprises a steroid, interferon, chemotherapy, phototherapy, radiation therapy, or a bone marrow transplant. In some embodiments, the subject has non-cancerous skin condition. In some embodiments, the non-cancerous skin condition comprises eczema. In some embodiments, the non-cancerous skin condition comprises psoriasis. In some embodiments, the non-cancerous skin condition comprises eczema. In some embodiments, the non-cancerous skin condition comprises atopic dermatitis or contact dermatitis. In some embodiments, the subject is a human. In some embodiments, the expression level is upregulated compared to a gene expression level of an equivalent gene from a control sample. In some embodiments, the expression level is downregulated compared to a gene expression level of an equivalent gene from a control sample. In some embodiments, the at least one target gene comprises a gene encoding an adapter protein, a gene encoding a tyrosine kinase, a gene encoding an interleukin, a gene encoding a transcription factor, a gene encoding a TNF receptor associated factor protein, a gene encoding a TNF, a gene encoding a TNF superfamily member, a gene encoding a saposin-like protein, a gene encoding a GTP-binding protein, a gene encoding a chromatin associated protein, a gene encoding a G-protein-coupled receptor, a gene encoding a transcriptional coactivator, a gene encoding a spermatogenesis protein, a gene encoding an actin-binding protein, a gene encoding a matrix metalloproteinase, a gene encoding a FYN-binding protein family member, a gene encoding a TEC kinase family member, a gene encoding a STAT, a gene encoding a TRAF3 interacting protein, a gene encoding a dynamin family member, a gene encoding a ubiquitin ligase, a gene encoding a thymocyte selection associated high mobility group box family member, a gene encoding a lymphoid enhancer binding factor family member, a gene encoding a C-C chemokine receptor type family member, a gene encoding an Oct binding factor family member, a gene encoding an gametocyte-specific family member, a gene encoding a plastin family member, a gene encoding a lymphocyte-specific protein tyrosine kinase family member, a gene encoding a member of the NEDD4 family of E3 HECT domain ubiquitin ligases, a gene encoding a C-C motif chemokine ligand family member, a gene encoding a chemokine, or a gene encoding a CXC chemokine, or a combination thereof. In some embodiments, the at least one target gene comprises a gene encoding a saposin-like protein, a gene encoding a FYN-binding protein family member, a gene encoding a TEC kinase family member, a gene encoding a STAT, a gene encoding a TRAF3 interacting protein, a gene encoding a CXC chemokine family member, or a combination thereof. In some embodiments, the at least one target gene comprises a gene encoding modulator of cell death, a gene encoding an antimicrobial, a gene encoding a cytokine, or a gene encoding a DNA-binding protein, or a combination thereof. In some embodiments, the at least one target gene comprises FYB, LEF1, GNLY, DMN3, ITK, IL26, STAT5, TRAF3IP3, TNFSF11, CCL27, CXCL8, CXCL9, CXCL10, TNF, or a combination thereof. In some embodiments, the at least one target gene comprises a gene encoding a microRNA. In some embodiments, the microRNA comprises miR-21, miR-29b, miR-155, miR-186, miR-214, or miR-221. Some embodiments further comprise detecting the presence at least one genotype of one more additional target genes known to be mutated in subjects with CTCL, in the nucleic acids or in a separate set of nucleic acids isolated from the skin sample. In some embodiments, the nucleic acids or the separate set of nucleic acids comprise DNA. In some embodiments, determining whether the subject has CTCL further comprises determining whether the subject has CTCL based on the presence of the at least one genotype. In some embodiments, the one or more additional target genes comprise TP53, ZEB1, ARID1A, DNMT3A, CDKN2A, FAS, STAT5B, PRKCQ, RHOA, DNMT3A, PLCG1, or NFKB2.

Disclosed herein, in certain embodiments, is a method of treating a subject with cutaneous T cell lymphoma (CTCL), comprising: identifying a subject suspected of having CTCL; isolating nucleic acids from a skin sample obtained from the subject by applying an adhesive patch to a skin region of the subject in a manner sufficient to adhere skin sample cells to the adhesive patch, and removing the adhesive patch from the skin sample in a manner sufficient to retain the adhered skin sample cells to the adhesive patch, wherein the skin sample cells comprise cells from the stratum corneum; detecting an expression level of at least one target gene known to be upregulated or downregulated in subjects with CTCL, by contacting the isolated nucleic acids with a set of probes that recognize the target gene, and detecting binding between the at least one target gene and the set of probes; determining whether the subject has CTCL based on the expression level of the at least one target gene; and administering a CTCL treatment to the subject when the subject is determined to have CTCL based on the expression level of the at least one target gene, and not administering the CTCL treatment to the subject when the subject is not determined to have CTCL based on the expression level of the at least one target gene. In some embodiments, the nucleic acids comprise mRNA. In some embodiments, the cells from the stratum corneum comprise T cells or components of T cells. In some embodiments, the cells from the stratum corneum comprise keratinocytes. In some embodiments, the skin sample does not comprise melanocytes. In some embodiments, the skin sample is obtained by applying a plurality of adhesive patches to the skin region of the subject in a manner sufficient to adhere skin sample cells to each of the adhesive patches, and removing each of the plurality of adhesive patches from the skin region in a manner sufficient to retain the adhered skin sample cells to each of the adhesive patches. In some embodiments, the skin region comprises a skin lesion. Some embodiments include determining that the subject has CTCL based on the expression level of the at least one target gene. Some embodiments include administering a CTCL treatment to the subject based on the determination of whether the subject has CTCL. In some embodiments, the CTCL treatment comprises a steroid, interferon, chemotherapy, phototherapy, radiation therapy, or a bone marrow transplant. In some embodiments, the skin sample comprises a CTCL skin lesion. In some embodiments, the CTCL comprises mycosis fungoides. In some embodiments, the CTCL comprises Sézary syndrome. In some embodiments, the subject is a human. In some embodiments, the expression level is upregulated compared to a gene expression level of an equivalent gene from a control sample. In some embodiments, the expression level is downregulated compared to a gene expression level of an equivalent gene from a control sample. In some embodiments, the at least one target gene comprises a gene encoding an adapter protein, a gene encoding a tyrosine kinase, a gene encoding an interleukin, a gene encoding a transcription factor, a gene encoding a TNF receptor associated factor protein, a gene encoding a TNF, a gene encoding a TNF superfamily member, a gene encoding a saposin-like protein, a gene encoding a GTP-binding protein, a gene encoding a chromatin associated protein, a gene encoding a G-protein-coupled receptor, a gene encoding a transcriptional coactivator, a gene encoding a spermatogenesis protein, a gene encoding an actin-binding protein, a gene encoding a matrix metalloproteinase, a gene encoding a FYN-binding protein family member, a gene encoding a TEC kinase family member, a gene encoding a STAT, a gene encoding a TRAF3 interacting protein, a gene encoding a dynamin family member, a gene encoding a ubiquitin ligase, a gene encoding a thymocyte selection associated high mobility group box family member, a gene encoding a lymphoid enhancer binding factor family member, a gene encoding a C-C chemokine receptor type family member, a gene encoding an Oct binding factor family member, a gene encoding an gametocyte-specific family member, a gene encoding a plastin family member, a gene encoding a lymphocyte-specific protein tyrosine kinase family member, a gene encoding a member of the NEDD4 family of E3 HECT domain ubiquitin ligases, a gene encoding a C-C motif chemokine ligand family member, a gene encoding a chemokine, or a gene encoding a CXC chemokine, or a combination thereof. In some embodiments, the at least one target gene comprises a gene encoding a saposin-like protein, a gene encoding a FYN-binding protein family member, a gene encoding a TEC kinase family member, a gene encoding a STAT, a gene encoding a TRAF3 interacting protein, a gene encoding a CXC chemokine family member, or a combination thereof. In some embodiments, the at least one target gene comprises FYN binding protein (FYB), IL2 inducible T-cell kinase (ITK), interleukin 26 (IL26), signal transducer and activator of transcription 5A (STAT5A), TRAF3 interacting protein 3 (TRAF3IP3), granulysin (GNLY), dynamin 3 (DNM3), or tumor necrosis factor superfamily member 11 (TNFSF11), or a combination thereof. In some embodiments, the at least one target gene comprises TOX, LEF1, CCR4, POU2AF1, GTSF1, PLS3, MMP12, LCK, or NEDD4L, or a combination thereof. In some embodiments, the at least one target gene comprises FYB, GNLY, ITK, STAT5, TRAF3IP3, CXCL10, CXCL8, or TNF, or a combination thereof. In some embodiments, the at least one target gene comprises a gene encoding a microRNA. In some embodiments, the microRNA comprises miR-21, miR-29b, miR-155, miR-186, miR-214, or miR-221. Some embodiments include detecting the presence at least one genotype of one more additional target genes known to be mutated in subjects with CTCL, in the nucleic acids or in a separate set of nucleic acids isolated from the skin sample. In some embodiments, the nucleic acids or the separate set of nucleic acids comprise DNA. In some embodiments, determining whether the subject has CTCL further comprises determining whether the subject has CTCL based on the presence of the at least one genotype. In some embodiments, the one or more additional target genes comprise TP53, ZEB1, ARID1A, DNMT3A, CDKN2A, FAS, STAT5B, PRKCQ, RHOA, DNMT3A, PLCG1, or NFKB2.

Disclosed herein, in certain embodiments, is a kit for determining the presence of cutaneous T cell lymphoma (CTCL) or a non-cancerous skin condition (e.g., eczema, psoriasis, atopic dermatitis, and/or contact dermatitis in a skin sample, comprising: an adhesive patch comprising an adhesive matrix configured to adhere skin sample cells from the stratum corneum of a subject; a nucleic acid isolation reagent; and a plurality of probes that recognize at least one target gene known to be upregulated or downregulated in subjects with CTCL.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates an example prediction system.

FIG. 2 illustrates an example skin diagnostic system.

FIG. 3 illustrates an example computing system that may implement various aspects of the prediction system.

FIG. 4 illustrates example operations for predicting a skin disease.

FIG. 5 illustrates example operations for training a machining learning model.

FIG. 6A is a chart depicting information regarding exemplary genes.

FIG. 6B is a chart depicting information regarding exemplary genes.

FIG. 7 illustrates exemplary gene biomarkers obtained from skin samples and tested for use as a diagnostic marker. The ‘V’ denotes genes displaying differential expression between CTCL tumor and normal skin samples, in FFPE tissues from biopsies, as reported in the respective study shown in the top row of the Figure.

FIG. 8 shows the expression results of 17 exemplary genes tested in lesional, non-lesional, and healthy unaffected control skin samples obtained non-invasively via adhesive patches.

FIG. 9 shows the expression levels of exemplary target genes normalized to housekeeping genes analyzed in parallel (shown as ΔCt (=Ct.target−Ct.HouseKeeping).

FIG. 10 shows fold change (FC) of the target genes from FIG. 3 in CTCL lesional skin samples compared to healthy unaffected controls (normal skin).

FIG. 11 depicts a gene expression analysis.

FIG. 12 depicts average gene expression data from lesional and non-lesional skin.

FIG. 13A is chart including gene expression data from lesional and non-lesional skin.

FIG. 13B is chart including gene expression data from lesional and non-lesional skin.

FIG. 14 depicts normalized gene expression in target genes in CTCL lesional skin samples compared to healthy unaffected controls (normal skin).

FIG. 15 depicts normalized gene expression in target genes in CTCL lesional skin samples compared to psoriasis lesional samples (non-cancerous skin condition).

FIG. 16 depicts normalized gene expression in target genes in psoriasis lesional skin samples compared to atopic dermatitis lesional samples.

### DETAILED DESCRIPTION OF THE DISCLOSURE

Aspects of the present disclosure involve systems and methods to process gene data with a diagnostic model to predict skin conditions of a tissue sample collected using an adhesive patch. The systems and methods described herein use the diagnostic model to provide a robust prediction of skin conditions, such as, cutaneous T cell lymphoma (CTCL), psoriasis, or atopic dermatitis. The diagnostic model leverages historical gene data relating to gene indicators of a skin condition to provide a prediction for a presence of a skin condition in a tissue sample collected using a non-invasive adhesive patch. This results in a more efficient platform that provides accurate predictions of skin conditions without requiring painful biopsies. Additional advantages of the presently disclosed technology will become apparent from the detailed description below.

In some embodiments, disclosed herein is a method of utilizing the expression level of genes in a gene classifier to determine the presence of CTCL. In some cases, the method comprises determining a change in the expression level of genes in a gene classifier, in which the change is compared to a gene expression level of an equivalent gene from a normal sample. In additional embodiments, disclosed herein is a method of determining whether a subject has CTCL based on the expression level of genes in a gene classifier. Some embodiments include the use of a genotype in determining the presence of the CTCL.

Disclosed herein, in some embodiments, are methods of determining the presence of cutaneous T cell lymphoma (CTCL) in a skin sample, comprising: identifying a subject suspected of having CTCL; isolating nucleic acids from a skin sample obtained from the subject by applying an adhesive patch to a skin region of the subject in a manner sufficient to adhere skin sample cells to the adhesive patch, and removing the adhesive patch from the skin sample in a manner sufficient to retain the adhered skin sample cells to the adhesive patch, wherein the skin sample cells comprise cells from the stratum corneum; and detecting an expression level of at least one target gene known to be upregulated or downregulated in subjects with CTCL, by contacting the isolated nucleic acids with a set of probes that recognize the target gene, and detecting binding between the at least one target gene and the set of probes. Some embodiments include the use of a genotype in determining the presence of the CTCL.

Disclosed herein, in some embodiments, are methods of treating a subject with cutaneous T cell lymphoma (CTCL), comprising: identifying a subject suspected of having CTCL; isolating nucleic acids from a skin sample obtained from the subject by applying an adhesive patch to a skin region of the subject in a manner sufficient to adhere skin sample cells to the adhesive patch, and removing the adhesive patch from the skin sample in a manner sufficient to retain the adhered skin sample cells to the adhesive patch, wherein the skin sample cells comprise cells from the stratum corneum; detecting an expression level of at least one target gene known to be upregulated or downregulated in subjects with CTCL, by contacting the isolated nucleic acids with a set of probes that recognize the target gene, and detecting binding between the at least one target gene and the set of probes; determining whether the subject has CTCL based on the expression level of the at least one target gene; and administering a CTCL treatment to the subject when the subject is determined to have CTCL based on the expression level of the at least one target gene, and not administering the CTCL treatment to the subject when the subject is not determined to have CTCL based on the expression level of the at least one target gene. Some embodiments include the use of a genotype in determining the presence of the CTCL.

Disclosed herein, in some embodiments, are kits for determining the presence of cutaneous T cell lymphoma (CTCL) in a skin sample, comprising: an adhesive patch comprising an adhesive matrix configured to adhere skin sample cells from the stratum corneum of a subject; a nucleic acid isolation reagent; and a plurality of probes. In some embodiments, the probes recognize at least one target gene known to be upregulated or downregulated in subjects with CTCL. In some embodiments, the probes recognize a genotype of at least one target gene known to be mutated in subjects with CTCL.

The kits and methods disclosed herein have several advantages over the prior art. An advantage of using target genes for identifying subjects with skin cancer such as CTCL, or for determining the presence of a skin cancer such as CTCL in a skin sample, is the relatively low cost of obtaining genetic data such as information about gene expression or genotypes. An advantage of using an adhesive tape to collect a skin sample is its non-invasiveness.

In some cases, gene expression data, such as measured amounts of mRNA of one or more target genes, are indicative of a skin cancer such as CTCL. Because mRNA levels do not always correlate with protein levels for a given gene, an existing method that measures protein levels would not render obvious the methods described herein. The usefulness of expression levels of the various genes and type of genes described herein is unexpected in light of such methods because of the unpredictability of whether mRNA levels and protein levels will always align. For example, in one instance a mRNA expression level for a gene may be increased in a CTCL skin lesion compared to a control sample while the protein level of the gene may be unchanged; or vice versa, a protein level may be increased or decreased in a CTCL skin lesion while an mRNA level for the same gene as the protein is unchanged.

### Method and System for Analyzing Gene Expression to Determine Skin Disorders

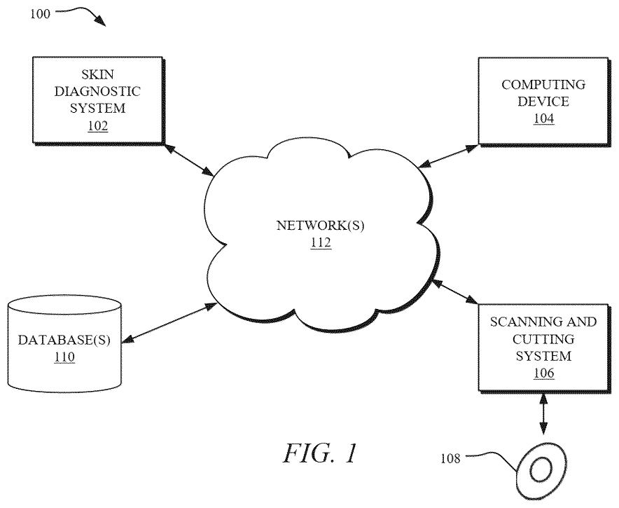

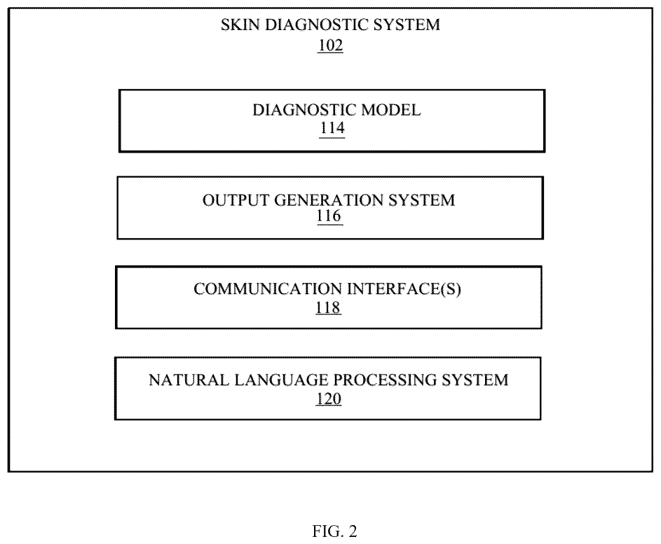

To begin a detailed description of an example system 100 for detecting a skin condition in a tissue sample, reference is made to FIGS. 1-5. In an implementation, the tissue sample is collected by a non-invasive skin sample collector, such as, an adhesive patch 108. In an implementation, the system 100 extracts gene data for target genes that correspond with the presence of a skin disease, such as, cutaneous T cell lymphoma (CTCL), psoriasis, or atopic dermatitis, from the tissue sample. The specific target genes are described in more detail below. The system 100 can include a skin diagnostic system 102, a computing device 104, a scanning and cutting system 106, and one or more databases. In an implementation, the skin diagnostic system is configured to receive inputs by an operator via one or more input systems using, for example, a computing device 104 to input text, audio, and/or interact with an interactive user interface displayed on one or more output systems of, for example, the computing device 104. In an implementation, gene data is received from the computing device 104 and/or one or more databases 110. The skin diagnostic system 102, the computing device 104, and the one or more databases 110 are configured to interact with one another via a network(s) 112. As illustrated in greater detail below, any and/or all of the skin diagnostic system 102, the computing device 104, the scanning and cutting system 106, and the one or more databases 110 may, in some instances, be special-purpose computing devices configured to perform specific functions.

The skin diagnostic system 102 can include one or more computing devices (e.g., servers, routers, user interface devices, internet telephony computing device, and the like) that store and/or retrieve data in the one or more databases 110, generate user interfaces, execute a diagnostic model 114, an output generation system 116, a natural language processing system 120 etc. by processing instructions. The skin diagnostic system 102 may include a communication interface(s) 118 that is able to communicate with the one or more input systems and one or more output systems of, for example, the computing device 104 and/or the scanning and cutting system 106, via the network(s) 112. For instance, the communication interface(s) 118 may be a network interface configured to support communication between the skin diagnostic system 102, the scanning and cutting system 106, and/or the computing device 104 with the network(s) 112. The one or more input systems and one or more output systems may be part of the computing device 104 and/or the scanning and cutting system 106 or separate from the computing device 104 and/or the scanning and cutting system 106. The skin diagnostic system 102 can be configured to train and maintain the diagnostic model 114 to execute the techniques, as discussed in greater detail below. The skin diagnostic system 102 can be configured to monitor and store (e.g., with appropriate permissions) data from the one or more databases 110 for further analysis and/or training of the diagnostic model 114. In an implementation, the skin diagnostic system 102 is configured to transmit output data to another computing device or database, such as the computing device 104 and/or the one or more databases 110. The skin diagnostic system 102, the computing device 104, the scanning and cutting system 106, and the one or more databases 110 are configured to interact with one another via the network(s) 112. In an implementation, the skin diagnostic system 102 is associated with an organization or entity, and the computing device is associated with a medical provider.

In an implementation, the computing device 104 includes one or more input systems and one or more output systems. For instance, the operator is able to input data to the skin diagnostic system 102 via one or more interactive user interfaces using the computing device 104. In an implementation, the input data is a natural language input by the user. The computing device 104 can be a smartphone, a tablet, a desktop computer, a laptop computer, or other personal computing device that may be used by an individual (e.g., the operator) to receive notification(s) and enter input data. In some instances, the computing device 104 may be used to display notifications and/or other alerts using graphical user interfaces.

In an implementation, the skin diagnostic system 102 includes instructions that direct and/or cause the natural language processing system 120 to execute processing techniques on the input data received by the one or more interactive user interfaces to generate processed input data. Based on the processed input data, the skin diagnostic system 102 generates an output, such as, for example, an indication of the presence of a skin condition, and performs one or more of the operations described herein. In an implementation, the natural language processing system 120 processes the input data using a large language model (LLM).

In an implementation, the skin diagnostic system 102 includes instructions that direct and/or cause the diagnostic model 114 to execute processing techniques on gene data received from the computing device 104, the scanning and cutting system 106 and/or the one or more databases 110 to generate prediction data associated with detection of a skin disease, such as cutaneous T cell lymphoma (CTCL), psoriasis, or atopic dermatitis. In an implementation, the gene data is extracted from a tissue sample that is obtained using the adhesive patch 108. In an implementation, the diagnostic model 114 utilizes machine learning techniques, such as, for example, one or more of a random forest model, a boosting model, a logit model, a lasso model, or any suitable machine learning model. In an implementation, nucleic acids are isolated from the tissue sample adhered to an adhesive patch, the tissue sample having been obtained from skin of a subject suspected of having a skin condition. In an implementation, a set of probes contact the isolated nucleic acids that recognize one or more target genes of interest implicated in the skin condition. The target genes of interest are described in more detail below. In an implementation, the diagnostic model 114 accounts for interactions of the target genes of interest. For instance, the gene data includes an amount of binding between the genes of interest and the set of probes. The presence of CTCL in the skin sample is identified based on the amount of binding between the target genes of interest and the set of probes relative to a control or threshold binding. In an implementation, a therapeutic agent is applied to the subject identified as having the skin condition.

In an implementation, the diagnostic model 114 is trained using training data. In an implementation, the training data includes historic gene data of successfully diagnosed skin conditions, thereby leveraging a large amount of historic gene data to accurately predict a skin condition in a tissue sample. The diagnostic model 114 is capable of processing a large amount of data to identify a correlation between the target genes and the skin condition. The diagnostic model 114 is able to be re-trained to increase accuracy. For instance, the diagnostic model 114 is re-trained based on successful or unsuccessful prediction of the skin condition.

In an implementation, the output generation system 116 is configured to perform one or more of the functions described herein. For example, the output generation system 116 may have instructions that direct and/or cause the output generation system 116 to generate a notification regarding the prediction data. For instance, the notification is audio, visual, and/or textual notification. In an implementation, the notification indicates the presence of the skin condition. In an implementation, the notification may be sent upon request and/or automatically to the computing device 104, such as, for example, e-mail, to indicate the prediction data. For instance, the notification may be sent upon completion of prediction, after approval by an operator, hourly, daily, weekly, monthly, etc. In another implementation, the notification indicates that the prediction data requires validation. In this implementation, the diagnostic model 114 is updated based on operator input with regards to the validation. In an implementation, the notification is presented via one or more interactive user interfaces, such as a report, a plot, and/or a bar graph, generated by the output generation system 116 and transmitted, via the communication interface(s) 118, to the computing device 104 for display by the output system of the computing device 104. In another implementation, the output generation system 116 indicates a recommendation regarding a treatment plan based on the prediction data. In another implementation, the output generation system 116 generates instructions to automatically develops a treatment plan for a therapeutic treatment based on the prediction data.

The network(s) 112 can be any combination of one or more of a cellular network such as a 3rd Generation Partnership Project (3GPP) network, a third generation (3G) network, a fourth generation (4G) network, a fifth generation (5G) network, a Long-Term Evolution (LTE), an LTE Advanced Network, a Global System for Mobile Communications (GSM) network, a Universal Mobile Telecommunications System (UMTS) network, and the like. Moreover, the network(s) 112 can include any type of network, such as the Internet, an intranet, a Virtual Private Network (VPN), a Voice over Internet Protocol (VOIP) network, a wireless network (e.g., Bluetooth), a cellular network, a satellite network, combinations thereof, etc. The network(s) 112 can include communications network components such as, but not limited to gateways routers, servers, and registrars, which enable communication across the network(s) 112. In one implementation, the communications network components include multiple ingress/egress routers, which may have one or more ports, in communication with the network(s) 112.

In an implementation, the adhesive patch 108 collects a combination of cells from epidermal lesional skin tissue as well as cells from an area the surrounding lesion. In an implementation, the adhesive patch 108 is an adhesive tape. The adhesive patch 108 can be of any suitable size such that it is sized larger than the desired collection area (e.g., lesion, mole) and is composed of a flexible material. In an implementation, the adhesive patch 108 is transparent or translucent such that the area of interest is visible. In another implementation, the adhesive patch 108 is opaque. In an implementation, the adhesive patch 108 includes an adhesive portion including an adhesive matrix that forms a collection area. In an implementation, the adhesive patch 108 includes a non-adhesive portion extending from at least a portion the periphery of the adhesive portion, thereby forming a handling area. For instance, the handling area may include a tab for applying and removing the adhesive patch 108 without coming in contact with the collection area. In an implementation, the collection area is a polyurethane carrier film. In an implementation, the adhesive matrix is comprised of a synthetic rubber compound or a styrene-isoprene-styrene (SIS) linear block copolymer compound. In an implementation, the adhesive patch 108 does not comprise latex, silicone, or both. In an implementation, the adhesive patch 108 is manufactured by applying an adhesive material as a liquid-solvent mixture to the collection area and subsequently removing the solvent. In an implementation, the adhesive matrix comprises one or more of acrylics, silicones and hydrocarbon rubbers (like butyl rubber, styrene-butadiene rubber, ethyl-vinyl acetate polymers, styrene-isoprene-butadiene rubbers), or combination thereof.

In an implementation, the scanning and cutting system 106 executes a software application to identify a delineation representing a border between cells of interest and a surrounding portion of the sample collector using image processing on image data captured using one or more image sensors. In an implementation, the delineation is drawn using a marking tool, such as, for example a pen or marker. In an implementation, the scanning and cutting system 106 cuts the adhesive patch 108 along the boundary formed by the delineation using a cutting device to separate the cells of interest from the surrounding portion. In an implementation, the scanning and cutting system 106 identifies the delineation and/or cuts along the delineation automatically without intervention of the operator. The cutting may be performed via mechanical cutting, plasma cutting, or laser cutting. In an implementation, laser cutting methods may include CO2, microjet, or fiber laser cutting.

In an implementation, the adhesive material and backing material are dissolved and membranes of the cells in the cells of interest are broken down using, for example, lysis, to expose the genetic material. The resulting cells are then cleaned and the remaining genetic material is recovered to generate gene data.

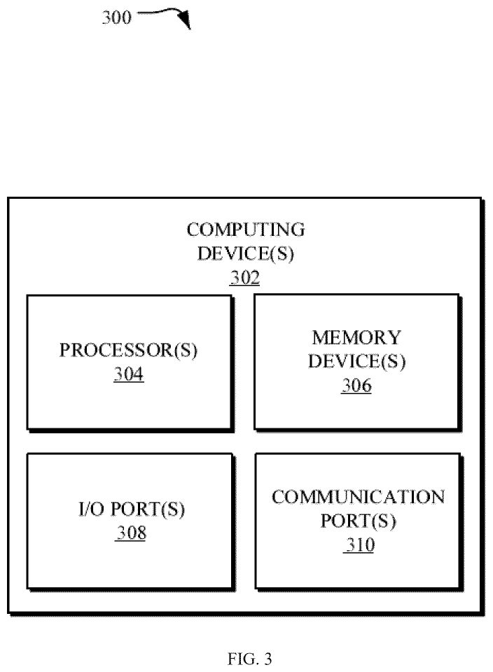

Turning to FIG. 3, a system 300 to process communication data can include one or more computing devices 302 for performing the techniques discussed herein. In one implementation, the one or more computing devices 302 include the computing device 104 and/or one or more servers of the skin diagnostic system 102 to generate and execute the diagnostic model 114, the output generation system 116, the natural language processing system 120, etc. as a software application and/or a module or algorithmic component of software.

In some instances, the computing device 302 can include a computer, a personal computer, a desktop computer, a laptop computer, a terminal, a workstation, a server device, a cellular or mobile phone, a mobile device, a smart mobile device a tablet, a wearable device (e.g., a smart watch, smart glasses, a smart epidermal device, etc.) a multimedia console, a television, an Internet-of-Things (IoT) device, a smart home device, a medical device, a virtual reality (VR) or augmented reality (AR) device, a vehicle (e.g., a smart bicycle, an automobile computer, etc.), and/or the like. The computing device 302 may be integrated with, form a part of, or otherwise be associated with the systems 100-300. It will be appreciated that specific implementations of these devices may be of differing possible specific computing architectures not all of which are specifically discussed herein but will be understood by those of ordinary skill in the art.

The computing device 302 may be a computing system capable of executing a computer program product to execute a computer process. Data and program files may be input to the computing device 302, which reads the files and executes the programs therein. Some of the elements of the computing device 302 include one or more processors 304, one or more memory devices 306, and/or one or more ports, such as input/output (IO) port(s) 308 and communication port(s) 310. Additionally, other elements that will be recognized by those skilled in the art may be included in the computing device 302 but are not explicitly depicted in FIG. 3 or discussed further herein. Various elements of the computing device 302 may communicate with one another by way of the communication port(s) 310 and/or one or more communication buses, point-to-point communication paths, or other communication means.

The processor 304 may include, for example, a central processing unit (CPU), a microprocessor, a microcontroller, a digital signal processor (DSP), and/or one or more internal levels of cache. There may be one or more processors 304, such that the processor 304 comprises a single central-processing unit, or a plurality of processing units capable of executing instructions and performing operations in parallel with each other, commonly referred to as a parallel processing environment.

The computing device 302 may be a conventional computer, a distributed computer, or any other type of computer, such as one or more external computers made available via a cloud computing architecture. The presently described technology is optionally implemented in software stored on the data storage device(s) such as the memory device(s) 306, and/or communicated via one or more of the I/O port(s) 308 and the communication port(s) 310, thereby transforming the computing device 302 in FIG. 3 to a special purpose machine for implementing the operations described herein. Moreover, the computing device 302, as implemented in the systems 100-300, receives various types of input data (e.g., the input data, well data, etc.) and transforms the input data through various stages of the data flow into new types of data files (e.g., embedding data). Moreover, these new data files are transformed further into the prediction data and sent to the computing device 104 to provide information regarding the predictions, which enables the computing device 302 to do something it could not do before-predicting, using a diagnostic model, presence of a skin condition in a tissue sample collected using a non-invasive method.

Additionally, the systems and operations disclosed herein represent an improvement to the technical field of machine learning processing. For instance, the skin diagnostic system 102 can generate accurate prediction data with less data and fewer computing resources without human intervention. Moreover, data can be leveraged from different data sources with varying levels of abstraction to provide a highly efficient and effective estimation of prediction data. These techniques are rooted in technology and could not have existed prior to the advent of machine learning analytics and simulation software.

The one or more memory device(s) 306 may include any non-volatile data storage device capable of storing data generated or employed within the computing device 302, such as computer executable instructions for performing a computer process, which may include instructions of both application programs and an operating system (OS) that manages the various components of the computing device 302. The memory device(s) 306 may include, without limitation, magnetic disk drives, optical disk drives, solid state drives (SSDs), flash drives, and the like. The memory device(s) 306 may include removable data storage media, non-removable data storage media, and/or external storage devices made available via a wired or wireless network architecture with such computer program products, including one or more database management products, web server products, application server products, and/or other additional software components. Examples of removable data storage media include Compact Disc Read-Only Memory (CD-ROM), Digital Versatile Disc Read-Only Memory (DVD-ROM), magneto-optical disks, flash drives, and the like. Examples of non-removable data storage media include internal magnetic hard disks, SSDs, and the like. The one or more memory device(s) 306 may include volatile memory (e.g., dynamic random access memory (DRAM), static random access memory (SRAM), etc.) and/or non-volatile memory (e.g., read-only memory (ROM), flash memory, etc.).

Computer program products containing mechanisms to effectuate the systems and methods in accordance with the presently described technology may reside in the memory device(s) 306 which may be referred to as machine-readable media. It will be appreciated that machine-readable media may include any tangible non-transitory medium that is capable of storing or encoding instructions to perform any one or more of the operations of the present disclosure for execution by a machine or that is capable of storing or encoding data structures and/or modules utilized by or associated with such instructions. Machine-readable media may include a single medium or multiple media (e.g., a centralized or distributed database, and/or associated caches and servers) that store the one or more executable instructions or data structures.

In some implementations, the computing device 302 includes one or more ports, such as the I/O port(s) 308 and the communication port(s) 310, for communicating with other computing, network, or vehicle computing devices. It will be appreciated that the I/O port 308 and the communication port 310 may be combined or separate and that more or fewer ports may be included in the computing device 302.

The I/O port 308 may be connected to an I/O device, or other device, by which information is input to or output from the computing device 302. Such I/O devices may include, without limitation, one or more input devices, output devices, and/or environment transducer devices.

In one implementation, the input devices convert a human-generated signal, such as, human voice, physical movement, physical touch or pressure, and/or the like, into electrical signals as input data into the computing device 302 via the I/O port 308. Similarly, the output devices may convert electrical signals received from the computing device 302 via the I/O port 308 into signals that may be sensed as output by a human, such as sound, light, and/or touch. The input device may be an alphanumeric input device, including alphanumeric and other keys for communicating information and/or command selections to the processor 304 via the I/O port 308. The input device may be another type of user input device including, but not limited to direction and selection control devices, such as a mouse, a trackball, cursor direction keys, a joystick, and/or a wheel; one or more sensors, such as a camera, a microphone, a positional sensor, an orientation sensor, an inertial sensor, and/or an accelerometer; and/or a touch-sensitive display screen (“touchscreen”). The output devices may include, without limitation, a display, a touchscreen, a speaker, a tactile and/or haptic output device, and/or the like. In some implementations, the input device and the output device may be the same device, for example, in the case of a touchscreen.

The environment transducer devices convert one form of energy or signal into another for input into or output from the computing device 302 via the I/O port 308. For example, an electrical signal generated within the computing device 302 may be converted to another type of signal, and/or vice-versa. In one implementation, the environment transducer devices sense characteristics or aspects of an environment local to or remote from the computing device 302, such as, light, sound, temperature, pressure, magnetic field, electric field, chemical properties, physical movement, orientation, acceleration, gravity, and/or the like.

In one implementation, the communication port 310 is connected to the network(s) 112 so the computing device 302 can receive network data useful in executing the methods and systems set out herein as well as transmitting information and network configuration changes determined thereby. Stated differently, the communication port 310 connects the computing device 302 to one or more communication interface devices configured to transmit and/or receive information between the computing device 302 and other devices by way of one or more wired or wireless communication networks or connections. Examples of such networks or connections include, without limitation, Universal Serial Bus (USB), Ethernet, Wi-Fi, Bluetooth®, Near Field Communication (NFC), and so on. One or more such communication interface devices may be utilized via the communication port 310 to communicate with one or more other machines, either directly over a point-to-point communication path, over a wide area network (WAN) (e.g., the Internet), over a local area network (LAN), over a cellular network (e.g., third generation (3G), fourth generation (4G), Long-Term Evolution (LTE), fifth generation (5G), etc.) or over another communication means. Further, the communication port 310 may communicate with an antenna or other link for electromagnetic signal transmission and/or reception.

In an example, the skin diagnostic system 102, the diagnostic model 114, the output generation system 116, the natural language processing system 120, etc., and/or other software, modules, services, and operations discussed herein may be embodied by instructions stored on the memory device(s) 306 and executed by the processor 304.

The system set forth in FIG. 3 is but one possible example of a computing device 302 or computer system that may be configured in accordance with aspects of the present disclosure. It will be appreciated that other non-transitory tangible computer-readable storage media storing computer-executable instructions for implementing the presently disclosed technology on a computing system may be utilized. In the present disclosure, the methods disclosed may be implemented as sets of instructions or software readable by the computing device 302.

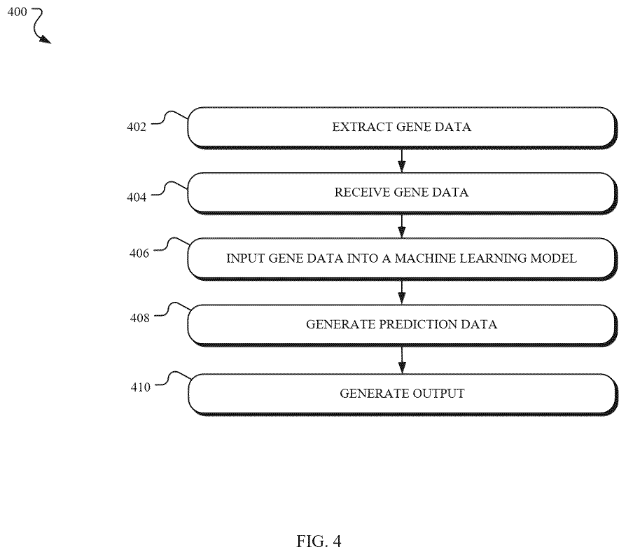

FIG. 4 depicts an example method 400 for detecting a skin condition, which can be performed by any of the systems 100-300 discussed herein. The method 400 can, in some instances, occur in real time.

At operation 402, the method 400 can extract gene data, via the scanning and cutting system 106 from a tissue sample collected using an adhesive patch 108.

At operation 404, the method 400 can receive the gene data at the skin diagnostic system 102.

At operation 406, the method 400 can input the gene data into the diagnostic model 114.

At operation 408, the method 400 can generate, via the diagnostic model 114, prediction data indicating presence of the skin disease in the tissue sample.

At operation 510, the method 400 can generate an output using the output generation system 116.

It is to be understood that the specific order or hierarchy of operations in the method depicted in FIG. 4 and throughout this disclosure are instances of example approaches and can be rearranged while remaining within the disclosed subject matter. For instance, any of the operations depicted in FIG. 4 may be omitted, repeated, performed in parallel, performed in a different order, and/or combined with any other of the operations depicted in FIG. 4 or discussed herein.

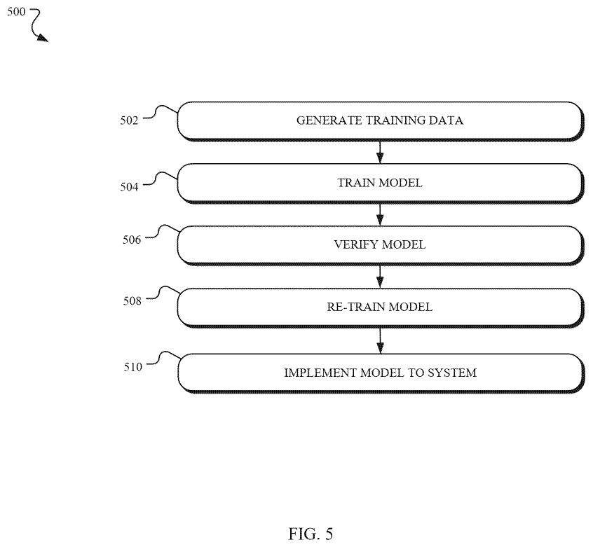

FIG. 5 depicts an example method 500 for training the diagnostic model 114, which can be performed by any of the systems 100-300 discussed herein. The method 500 can, in some instances, occur in real time.

At operation 502, the method 500 can generate training data based on historic gene data.

At operation 504, the method 500 can train the diagnostic model 114 using the training data.

At operation 506, the method 500 can verify the diagnostic model 114. In an implementation, the diagnostic model is verified based on a comparison of prediction data generated by the diagnostic model 114 with known data. The transformer is verified when the prediction data is within an error threshold of the known data.

At operation 508, the method 500 can re-train the diagnostic model based on the verification.

At operation 510, the method 500 can implement the transformer to the prediction system 100.

In an implementation, the method 500 is repeated until an acceptable error threshold is reached. It is to be understood that the specific order or hierarchy of operations in the method depicted in FIG. 5 and throughout this disclosure are instances of example approaches and can be rearranged while remaining within the disclosed subject matter. For instance, any of the operations depicted in FIG. 5 may be omitted, repeated, performed in parallel, performed in a different order, and/or combined with any other of the operations depicted in FIG. 5 or discussed herein.

### Target Genes, Gene Classifiers, and Methods of Use

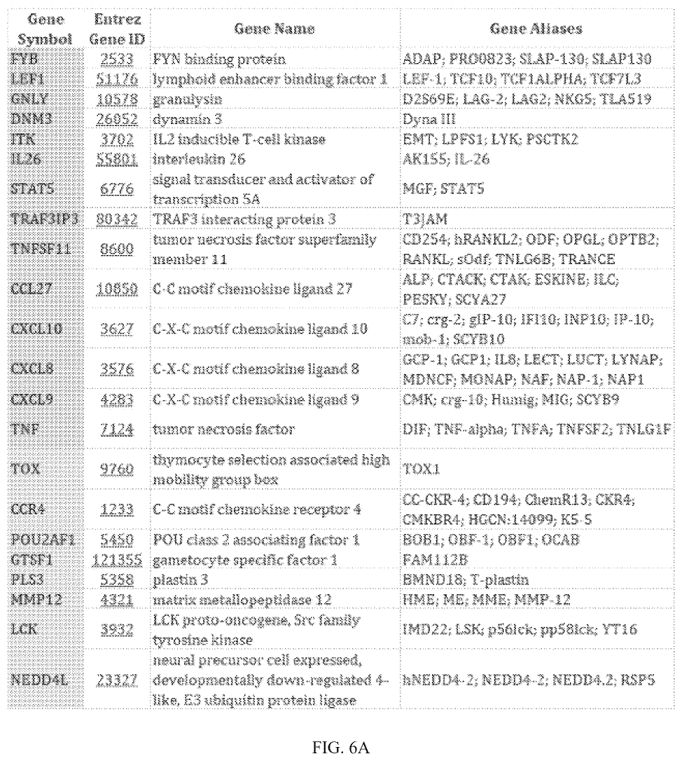

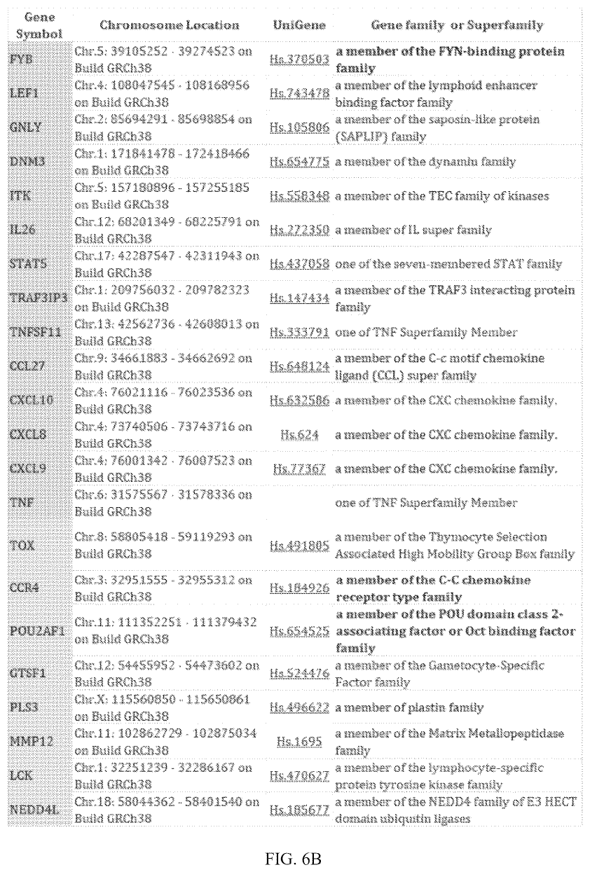

Disclosed herein, in some embodiments, are methods that include measuring, detecting, or using a target gene. For example, some embodiments relate to a method of determining the presence of a skin condition, such as skin cancer such as a cutaneous T cell lymphoma (CTCL), non-Hodgkin lymphoma, melanoma, basal cell carcinoma (BCC), Merkel cell carcinoma, sebaceous carcinoma, and/or squamous cell carcinoma (SCC), and/or other skin conditions, such as eczema, psoriasis, atopic dermatitis, lupus, rubeola, acne, hemangioma, candidiasis, impetigo, shingles, leprosy, Crohn's disease, inflammatory dermatoses, bullous disease, solar lentigo, dermatofibrosarcoma protuberans, and/or dysplastic nevi based on a presence or expression level of the target gene, and/or based on a mutation in the target gene. Some embodiments relate to a method of identifying a subject with the skin cancer (e.g. CTCL) based on a presence or expression level of the target gene, and/or based on a mutation in the target gene. Some embodiments include determining the presence of the skin cancer (e.g. CTCL) based on a presence or expression level of the target gene. Some embodiments include determining the presence of the skin cancer (e.g. CTCL) based on a mutation in the target gene. Some embodiments include the use of multiple target genes. Some embodiments include a target gene described in FIGS. 6A-6B. In some embodiments, the target genes described herein are used in any method described herein.

Disclosed herein, in some embodiments, are methods that include measuring, detecting, or using a target gene. For example, some embodiments relate to a method of determining the presence of a non-cancerous skin condition such as eczema, psoriasis, atopic dermatitis, contact dermatitis lupus, rubeola, acne, hemangioma, candidiasis, impetigo, shingles, leprosy, Crohn's disease, inflammatory dermatoses, bullous disease, solar lentigo, dermatofibrosarcoma protuberans, and/or dysplastic nevi based on a presence or expression level of the target gene, and/or based on a mutation in the target gene. Some embodiments relate to a method of identifying a subject with the skin condition (e.g., eczema, psoriasis, atopic dermatitis, or contact dermatitis) based on a presence or expression level of the target gene, and/or based on a mutation in the target gene. Some embodiments include determining the presence of the skin condition (e.g., eczema, psoriasis, atopic dermatitis, or contact dermatitis) based on a presence or expression level of the target gene. Some embodiments include determining the presence of the skin condition (e.g., eczema, psoriasis, atopic dermatitis, or contact dermatitis) based on a mutation in the target gene. Some embodiments include the use of multiple target genes. Some embodiments include a target gene described in FIGS. 6A-6B. In some embodiments, the target genes described herein are used in any method described herein.

Disclosed herein, in some embodiments, are methods that utilize the non-transitory computer readable media, systems, and methods described above that receive gene data, which can be extracted from the tissue sample collected from adhesive skin sample collectors described herein. In some embodiments, the gene data can be inputted into one or more diagnostic models to generate prediction data with one or more diagnostic models. In some embodiments, the non-transitory computer readable media, systems, and methods described herein can generate output data that can indicate whether the tissue sample includes a skin disease (e.g., CTCL or a non-cancerous skin condition).

In some embodiments, the target gene can encode an adapter protein. In some embodiments, the adapter protein is a cytosolic adapter protein. In some embodiments, the adapter protein acts as an adapter protein in a signaling cascade such as a FYN and/or LCP2 signaling cascade. In some embodiments, the adapter protein is expressed by platelets, T cells, natural killer cells, myeloid cells, and/or dendritic cells. In some embodiments, the adapter protein is involved in cell motility, proliferation, activation, and cytokine production. A non-limiting example of such an adapter protein is the protein encoded by FYB. Some embodiments include measuring or detecting the presence or an amount of an mRNA encoding one or more adaptor proteins.

In some embodiments, the adapter protein is a FYN-binding protein family member. In some embodiments, the target gene encodes a FYN-binding protein family member. In some embodiments, the FYN-binding protein family member is FYB. Some embodiments include measuring or detecting the presence or an amount of mRNA encoding one or more FYN-binding protein family members.

In some embodiments, the target gene can encode an enzyme. In some embodiments, the enzyme is a kinase. In some embodiments, the target gene encodes a kinase. In some embodiments, the kinase is a tyrosine kinase. In some embodiments, the target gene encodes a tyrosine kinase. Examples of tyrosine kinases include but are not limited to proteins encoded by ITK and LCK. Some embodiments include multiple genes encoding tyrosine kinases as target genes. In some embodiments, the tyrosine kinases include ITK and LCK. Some embodiments include measuring or detecting the presence or an amount of an mRNA encoding one or more tyrosine kinase.

In some embodiments, the tyrosine kinase can be an intracellular tyrosine kinase. In some embodiments, the tyrosine kinase is thought to play a role in T-cell proliferation and differentiation. In some embodiments, the tyrosine kinase is expressed in T-cells. A non-limiting example of such a tyrosine kinase is the protein encoded by ITK.

In some embodiments, the tyrosine kinase can be a member of the TEC family of kinases. In some embodiments, the target gene encodes a TEC kinase family member. In some embodiments, the TEC family member is the protein encoded by ITK. Some embodiments include measuring or detecting the presence or an amount of mRNA encoding one or more TEC kinase family members.

In some embodiments, the tyrosine kinase can be a lymphocyte-specific protein tyrosine kinase family member. In some embodiments, the target gene encodes a lymphocyte-specific protein tyrosine kinase family member. In some embodiments, the lymphocyte-specific protein tyrosine kinase family member is a non-receptor tyrosine kinase. In some embodiments, the lymphocyte-specific protein tyrosine kinase family member is a member of the Src family of protein tyrosine kinases. In some embodiments, the lymphocyte-specific protein tyrosine kinase family member is expressed in T cells. In some embodiments, the lymphocyte-specific protein tyrosine kinase family member is anchored to a plasma membrane. In some embodiments, the lymphocyte-specific protein tyrosine kinase family member associates with cytoplasmic tails of CD4 or CD8 co-receptors. In some embodiments, the lymphocyte-specific protein tyrosine kinase family member phosphorylates an intracellular chain of CD3 or ζ-chains of a TCR complex. In some embodiments, the lymphocyte-specific protein tyrosine kinase family member phosphorylates ZAP-70. In some embodiments, upon T cell activation, the lymphocyte-specific protein tyrosine kinase family member translocates from outside a lipid raft to inside the lipid raft and activates Fyn. A non-limiting example of such a lymphocyte-specific protein tyrosine kinase family member is the protein encoded by LCK. Some embodiments include measuring or detecting the presence or an amount of mRNA encoding one or more lymphocyte-specific protein tyrosine kinase family members.

In some embodiments, the enzyme can be a matrix metalloproteinasc. In some embodiments, the target gene encodes a matrix metalloproteinase. In some embodiments, the matrix metalloproteinase can be a member of the peptidase M10 family of matrix metalloproteinases. In some embodiments, the matrix metalloproteinase is involved in the breakdown of an extracellular matrix. A non-limiting example of such a matrix metalloproteinase is the protein encoded by MMP12. Some embodiments include measuring or detecting the presence or an amount of mRNA encoding one or more matrix metalloproteinases.

In some embodiments, the enzyme can be a ubiquitin ligase. In some embodiments, the target gene encodes a ubiquitin ligase. In some embodiments, the ubiquitin ligase is an E3 ubiquitin ligase. In some embodiments, the ubiquitin ligase comprises a HECT domain. In some embodiments, the ubiquitin ligase is a member of the Nedd4 family of HECT domain E3 ubiquitin ligases. In some embodiments, the ubiquitin ligase ubiquitinates an epithelial sodium channel, a Na⁺—Cl⁻ co-transporter, or a voltage gated sodium channel. In some embodiments, the ubiquitin ligase comprises a Ca²⁺-phospholipid binding domain. In some embodiments, the ubiquitin ligase comprises a WW protein-protein interaction domain. A non-limiting example of such a ubiquitin ligase is the protein encoded by NEDD4L. Some embodiments include measuring or detecting the presence or an amount of mRNA encoding one or more ubiquitin ligases as described herein.

In some embodiments, the ubiquitin ligase can be a member of the NEDD4 family of E3 HECT domain ubiquitin ligases. In some embodiments, the target gene encodes a member of the NEDD4 family of E3 HECT domain ubiquitin ligases. In some embodiments, the member of the NEDD4 family of E3 HECT domain ubiquitin ligases is NEDD4L. Some embodiments include measuring or detecting the presence or an amount of mRNA encoding one or more members of the NEDD4 family of E3 HECT domain ubiquitin ligases.

In some embodiments, the enzyme can be a guanosine triphosphate (GTP)-binding protein. In some embodiments, the target gene encodes a GTP-binding protein. In some embodiments, the GTP-binding protein is a GTPase. In some embodiments, the GTP-binding protein is involved in actin-membrane a process such as membrane budding. In some embodiments, the GTP-binding protein associates with microtubules. In some embodiments, the GTP-binding protein is involved in vesicular transport. A non-limiting example of such a GTP-binding protein is the protein encoded by DNM3. Some embodiments include measuring or detecting the presence or an amount of mRNA encoding one or more GTP-binding proteins.

In some embodiments, the enzyme can be a nitric oxide synthase (NOS; e.g., inducible nitric oxide synthase), such as NOS2. In some embodiments, the target gene encodes a NOS. In some embodiments, the NOS is NOS2. In some embodiments, the NOS2 is generating nitric oxide. Some embodiments include measuring or detecting the presence or an amount of mRNA encoding one or more NOS enzymes.

In some embodiments, the GTP-binding protein can be a dynamin. In some embodiments, the target gene encodes a dynamin. In some embodiments, the dynamin is DNM3. Some embodiments include measuring or detecting the presence or an amount of mRNA encoding one or more dynamins.

In some embodiments, the target gene can encode a member of a TNF receptor associated factor protein family. In some embodiments, the TNF receptor associated factor protein family member is TRAF3IP3. Some embodiments include measuring or detecting the presence or an amount of mRNA encoding one or more TNF receptor associated factor proteins.

In some embodiments, the member of a TNF receptor associated factor protein family can be a TRAF3 interacting protein. In some embodiments, the target gene encodes a TRAF3 interacting protein. In some embodiments, the TRAF3 interacting protein mediates growth. In some embodiments, the TRAF3 interacting protein modulates the c-Jun N-terminal kinas signal transduction pathway. In some embodiments, the TRAF3 interacting protein interacts with a multi-protein assembly containing a phosphatase 2A catalytic subunit. A non-limiting example of such a TRAF3 interacting protein is the protein encoded by TRAF3IP3. Some embodiments include measuring or detecting the presence or an amount of mRNA encoding one or more TRAF3 interacting proteins.

In some embodiments, the target gene can encode a cytokine. Examples of cytokines include but are not limited to proteins encoded by TNFSF11, IL26, CCL27, CXCL8, CXCL9, CXCL10, and TNF. Examples of cytokines include but are not limited to chemokines and interleukins. Some embodiments include multiple genes encoding cytokines as target genes. In some embodiments, the cytokines include TNFSF11. In some embodiments, the cytokines include IL26. In some embodiments, the cytokines include CCL27. In some embodiments, the cytokines include CXCL8. In some embodiments, the cytokines include CXCL9. In some embodiments, the cytokines include CXCL10. In some embodiments, the cytokines include TNF. In some embodiments, the cytokines include 1, 2, 3, 4, 5, 6, or 7, or a range defined by any of the aforementioned integers, of TNFSF11, IL26, CCL27, CXCL8, CXCL9, CXCL10, or TNF. Some embodiments include measuring or detecting the presence or an amount of an mRNA encoding one or more cytokines.

In some embodiments, the cytokine can be a TNF superfamily member. In some embodiments, the target gene encodes a TNF superfamily member. In some embodiments, the TNF superfamily member is involved in inflammation. In some embodiments, the TNF superfamily member is part of an acute phase inflammatory reaction. In some embodiments, the TNF superfamily member comprises a TNF domain. In some embodiments, the TNF superfamily member is a pyrogen. In some embodiments, the TNF superfamily member induces apoptosis. In some embodiments, the TNF superfamily member is secreted by a macrophage. In some embodiments, the TNF superfamily member binds TNFRSF1A/TNFR1 and/or TNFRSF1B/TNFBR. A non-limiting example of such a TNF superfamily member is TNFα, the protein encoded by TNF. In some embodiments, the cytokine is TNFα (encoded by TNF). Some embodiments include measuring or detecting the presence or an amount of mRNA encoding one or more TNF superfamily members.

In some embodiments, the cytokine can be a modulator of cell death. In some embodiments, the cell death comprises or consists of apoptosis In some embodiments, the target gene encodes a modulator of cell death. Examples of cell death modulators include but are not limited to proteins encoded by IL26, GNLY, TNFSF11, and TNF. In some embodiments, the modulator of cell death is encoded by IL26. In some embodiments, the modulator of cell death is encoded by GNLY. In some embodiments, the modulator of cell death is encoded by TNFSF11. In some embodiments, the modulator of cell death is encoded by TNF. Some embodiments include multiple genes encoding modulators of cell death as target genes. In some embodiments, the modulators of cell death include proteins encoded by IL26 and GNLY. In some embodiments, the modulators of cell death include proteins encoded by GNLY and TNFSF11. In some embodiments, the modulators of cell death include proteins encoded by IL26 and TNFSF11. In some embodiments, the modulators of cell death include proteins encoded by IL26, GNLY, and TNFSF11. In some embodiments, the modulators of cell death include proteins encoded by TNF, IL26 and GNLY. In some embodiments, the modulators of cell death include proteins encoded by TNF, GNLY and TNFSF11. In some embodiments, the modulators of cell death include proteins encoded by TNF, IL26 and TNFSF11. In some embodiments, the modulators of cell death include proteins encoded by TNF, IL26, GNLY, and TNFSF11. Some embodiments include measuring or detecting the presence or an amount of an mRNA encoding one or more modulators of cell death as described herein.

In some embodiments, the cytokine can be a chemokine. In some embodiments, the target gene encodes a chemokine. Examples of chemokines include but are not limited to proteins encoded by CCL27, CXCL8, CXCL9, and CXCL10. Some embodiments include multiple genes encoding chemokines as target genes. In some embodiments, the chemokines include CCL27 and CXCL8. In some embodiments, the chemokines include CCL27 and CXCL9. In some embodiments, the chemokines include CCL27 and CXCL10. In some embodiments, the chemokines include CXCL8, CXCL9, and CXCL10. In some embodiments, the chemokines include CCL27, CXCL8, and CXCL9. In some embodiments, the chemokines include CCL27, CXCL8, and CXCL10. In some embodiments, the chemokines include CCL27, CXCL9, and CXCL10. In some embodiments, the chemokines include CCL27, CXCL8, CXCL9, and CXCL10. In some embodiments, the chemokines include CXCL8 and CXCL9. In some embodiments, the chemokines include CXCL8 and CXCL10. In some embodiments, the chemokines include CXCL9 and CXCL10. Some embodiments include measuring or detecting the presence or an amount of an mRNA encoding one or more chemokines.

In some embodiments, the chemokine can be a C-C motif chemokine ligand family member. In some embodiments, the target gene encodes a C-C motif chemokine ligand family member. Some embodiments include measuring or detecting the presence or an amount of mRNA encoding one or more C-C motif chemokine ligand family members. In some embodiments, the C-C motif chemokine ligand family member is CCL27.

In some embodiments, the C-C motif chemokine ligand family member can be a CC cytokine. In some embodiments, the target gene encodes a CC cytokine. In some embodiments, the CC cytokine is clustered on the p-arm of chromosome 9. In some embodiments, the CC chemokine is secreted. In some embodiments, the CC cytokine is involved in an immunoregulatory or inflammatory process. In some embodiments, the CC cytokine comprises two adjacent cysteines. In some embodiments, the CC cytokine is chemotactic for skin-associated memory T lymphocytes. In some embodiments, the CC cytokine is associated with homing of memory T lymphocytes to the skin. In some embodiments, the CC cytokine plays a role in skin inflammation. In some embodiments, the CC cytokine binds a chemokine receptor such as CCR10. A non-limiting example of such a CC cytokine is the protein encoded by CCL27. Some embodiments include measuring or detecting the presence or an amount of mRNA encoding one or more CC cytokines.

In some embodiments, the chemokine can be a CXC chemokine. In some embodiments, the target gene encodes a CXC chemokine. Examples of CXC chemokines include but are not limited to proteins encoded by CXCL8, CXCL9, and CXCL10. Some embodiments include multiple genes encoding CXC chemokines as target genes. In some embodiments, the CXC chemokines include CXCL8 and CXCL9. In some embodiments, the CXC chemokines include CXCL8 and CXCL10. In some embodiments, the CXC chemokines include CXCL9 and CXCL10. In some embodiments, the CXC chemokines include CXCL8, CXCL9, and CXCL10. Some embodiments include measuring or detecting the presence or an amount of an mRNA encoding one or more CXC chemokines.

In some embodiments, the CXC chemokine can be produced by a macrophage. In some embodiments, the CXC chemokine is produced by an epithelial cell, airway smooth muscle cell, or an endothelial cell. In some embodiments, the CXC chemokine is stored in a storage vesicle such as a Weibel-Palade body by a cell such as an endothelial cell. In some embodiments, the CXC chemokine is initially produced as a precursor peptide which undergoes cleavage. In some embodiments, the CXC chemokine binds heparin. In some embodiments, the CXC chemokine binds a receptor such as a GPCR, or a serpentine receptor such as CXCR1 or CXCR2. In some embodiments, the CXC chemokine is secreted. In some embodiments, the CXC chemokine mediates an immune reaction such as an innate immune reaction. In some embodiments, the CXC chemokine mediates activation of a neutrophil. In some embodiments, the CXC chemokine mediates migration of neutrophils into tissue from peripheral blood. A non-limiting example of such a CXC chemokine is the protein encoded by CXCL8.

In some embodiments, the CXC chemokine can be a monokine induced by gamma interferon (IFN-γ). In some embodiments, the CXC chemokine plays a role in chemotaxis. In some embodiments, the CXC chemokine promotes differentiation or multiplication of a leukocyte. In some embodiments, the CXC chemokine causes tissue extravasion. In some embodiments, the CXC chemokine mediates lymphocytic infiltration to the focal sites. In some embodiments, the CXC chemokine suppresses tumor growth. In some embodiments, the CXC chemokine interacts with CXCR3. In some embodiments, the CXC chemokine elicits a chemotactic function by interacting with CXCR3. In some embodiments, the CXC chemokine is involved in T cell trafficking. In some embodiments, the CXC chemokine is an antimicrobial. In some embodiments, the CXC chemokine is a chemoattractant for lymphocytes. In some embodiments, the CXC chemokine is not a chemoattractant for neutrophils. A non-limiting example of such a CXC chemokine is the protein encoded by CXCL9.

In some embodiments, the CXC chemokine can be a chemoattractant. In some embodiments, the CXC chemokine is an antimicrobial. In some embodiments, the CXC chemokine interacts with CXCR3. In some embodiments, the CXC chemokine elicits a chemotactic function by interacting with CXCR3. A non-limiting example of such a CXC chemokine is the protein encoded by CXCL10. Some embodiments include measuring or detecting the presence or an amount of mRNA encoding one or more CXC chemokines.

In some embodiments, the cytokine can be an interleukin. In some embodiments, the target gene encodes an interleukin. Examples of interleukins include but are not limited to proteins encoded by IL26 and CXCL8. Some embodiments include multiple genes encoding interleukins as target genes. In some embodiments, the interleukins include IL26 and CXCL8.

In some embodiments, the interleukin can be expressed in a T cell such as a herpesvirus-transformed T cell. In some embodiments, the interleukin is a TH17-cell derived interleukin. In some embodiments, the TH17-cell derived cytokine is IL-26. In some embodiments, the interleukin induces phosphorylation of a transcription factor such as STAT1 or STAT3. In some embodiments, the interleukin enhances the secretion of another interleukin such as IL-10 or IL-8. In some embodiments, the interleukin is an antimicrobial. In some embodiments, the interleukin promotes sensing of bacterial and host cell death. In some embodiments, the interleukin is a cationic amphipathic protein. In some embodiments, the interleukin kills extracellular bacteria by membrane-pore formation. In some embodiments, the interleukin complexes with bacterial DNA or self-DNA released by dying bacterial or host cells. In some embodiments, the interleukin activates a Toll-like receptor such as Toll-like receptor 9. In some embodiments, the interleukin activates an IL-26 receptor. A non-limiting example of such an interleukin is the protein encoded by IL26. Some embodiments include measuring or detecting the presence or an amount of mRNA encoding one or more interleukins.

In some embodiments, the chemokine can be an antimicrobial. In some embodiments, the interleukin is an antimicrobial. In some embodiments, the target gene encodes an antimicrobial. Examples of antimicrobials include but are not limited to proteins encoded by IL26 and GNLY. In some embodiments, the antimicrobial has an anti-tumor effect, or is also an anti-tumor protein. Some embodiments include multiple genes encoding antimicrobials as target genes. In some embodiments, the antimicrobials include IL26 and GNLY. Some embodiments include measuring or detecting the presence or an amount of an mRNA encoding one or more antimicrobials.

In some embodiments, the chemokine can be an interleukin. In some embodiments, the interleukin is a member of the CXC chemokine family. In some embodiments, the interleukin is CXCL8.

In some embodiments, the interleukin can be a member of the IL-10 family of cytokines. In some embodiments, the member of the IL-10 family of cytokines is IL26 and/or IL22. In some embodiments, the interleukin can include IL13, IL17A, and/or IL23A. In some embodiments, the interleukin may be an interleukin receptor, such as IL4R.

In some embodiments, the target gene can encode a DNA-binding protein. Examples of genes encoding DNA-binding proteins include but are not limited to IL26, STAT5A, TOX, and LEF1. Some embodiments include multiple genes encoding DNA-binding proteins as target genes. In some embodiments, the DNA-binding proteins include IL26 and STAT5A. In some embodiments, the DNA-binding proteins include IL26 and TOX. In some embodiments, the DNA-binding proteins include IL26 and LEF1. In some embodiments, the DNA-binding proteins include STAT5A and TOX. In some embodiments, the DNA-binding proteins include STAT5A and LEF1. In some embodiments, the DNA-binding proteins include TOX and STAT5A. In some embodiments, the DNA-binding proteins include IL26, STAT5A, and TOX. In some embodiments, the DNA-binding proteins include IL26, STAT5A, and LEF1. In some embodiments, the DNA-binding proteins include IL26, TOX, and LEF1. In some embodiments, the DNA-binding proteins include STAT5A, TOX, and LEF1. In some embodiments, the DNA-binding proteins include IL26, STAT5A, TOX, and LEF1. Some embodiments include measuring or detecting the presence or an amount of an mRNA encoding one or more DNA-binding proteins.

In some embodiments, the DNA-binding protein can be a transcription factor. In some embodiments, the target gene encodes a transcription factor. Examples of transcription factors include but are not limited to proteins encoded by STAT5A and LEF1. Some embodiments include multiple genes encoding transcription factors as target genes. In some embodiments, the transcription factors include STAT5A and LEF1. Some embodiments include measuring or detecting the presence or an amount of an mRNA encoding one or more transcription factors.

In some embodiments, the transcription factor can be a signal transducer and activator of transcription (STAT) family member. In some embodiments, the target gene encodes a STAT family member. In some embodiments, the STAT family member includes an N-terminal domain, a coiled-coil domain, a DNA binding domain, a linker domain, a Src Homology 2 domain, and/or a transcriptional activation domain. In some embodiments, the STAT family member is phosphorylated by a receptor associated kinase. In some embodiments, the STAT family member forms homo- or heterodimers that translocate to the cell nucleus upon phosphorylation. In some embodiments, the STAT family member mediates the response of a cell ligand such as IL2, IL3, IL7 GM-CSF, erythropoietin, thrombopoietin, or a growth hormone. A non-limiting example of such a STAT family member is the protein encoded by STAT5A. Some embodiments include measuring or detecting the presence or an amount of mRNA encoding one or more STAT family members.

In some embodiments, the transcription factor can be a lymphoid enhancer binding factor family member. In some embodiments, the target gene encodes a lymphoid enhancer binding factor family member. In some embodiments, the lymphoid enhancer binding factor family member is a nuclear protein. In some embodiments, the lymphoid enhancer binding factor family member is expressed in pre-B cells and/or in T cells. In some embodiments, the lymphoid enhancer binding factor family member binds to a T-cell receptor-alpha enhancer. In some embodiments, the lymphoid enhancer binding factor family member binding to the T-cell receptor-alpha enhancer increases enhancer activity. In some embodiments, the lymphoid enhancer binding factor family member is a member of a family of regulatory proteins that share homology with high mobility group protein-1. A non-limiting example of such a lymphoid enhancer binding factor family member is the protein encoded by LEF1. Some embodiments include measuring or detecting the presence or an amount of mRNA encoding one or more lymphoid enhancer binding factor family members.

In some embodiments, the target gene can encode a transcriptional coactivator. In some embodiments, the transcriptional coactivator is expressed in B-cell lymphocytes. In some embodiments, the transcriptional coactivator controls expression of immunoglobulin, CD20, CRISP-3, or CD36. A non-limiting example of such a transcriptional coactivator is the protein encoded by POU2AF1. Some embodiments include measuring or detecting the presence or an amount of mRNA encoding one or more transcriptional coactivators.

In some embodiments, the transcriptional coactivator can be a POU domain class 2-associating factor family member. In some embodiments, the target gene encodes a POU domain class 2-associating factor family member. In some embodiments, the POU domain class 2-associating factor family member is an Oct binding factor family member. In some embodiments, the POU domain class 2-associating factor family member is POU2AF1. Some embodiments include measuring or detecting the presence or an amount of mRNA encoding one or more POU domain class 2-associating factor family members.

In some embodiments, the target gene can encode a saposin-like protein family member. In some embodiments, the saposin-like protein family member is present in cytotoxic granules of cytolytic T cells or natural killer (NK) cells and is released from the granules upon antigen stimulation. In some embodiments, the saposin-like protein family member is an antimicrobial. In some embodiments, the saposin-like protein family member induces cell death (e.g. apoptosis) in target cell. A non-limiting example of such a saposin-like protein family member is the protein encoded by GNLY. Some embodiments include measuring or detecting the presence or an amount of mRNA encoding one or more saposin-like protein family members as described herein.

In some embodiments, the target gene can encode a tumor necrosis factor (TNF) superfamily member. In some embodiments, the TNF superfamily member regulates apoptosis. In some embodiments, the TNF superfamily member is a ligand for a receptor such as receptor activator of nuclear factor κB (RANK) or osteoprotegerin. In some embodiments, the TNF superfamily member controls cell proliferation, for example by modifying protein levels of Id4, Id2 or cyclin D1. In some embodiments, the TNF superfamily member functions as a factor in osteoclast differentiation or activation. In some embodiments, the TNF superfamily member is a cell survival factor. In some embodiments, the TNF superfamily member is involved in the regulation of T cell-dependent immune response. In some embodiments, the TNF superfamily member activates AKT/PKB, for example through a signaling complex involving SRC kinase and tumor necrosis factor receptor-associated factor (TRAF) 6. A non-limiting example of such a TNF superfamily member is the protein encoded by TNFSF11.

In some embodiments, the target gene can encode a chromatin associated protein. In some embodiments, the chromatin associated protein binds DNA in a sequence-specific manner binding protein. In some embodiments, the chromatin associated protein induces a bend in DNA bound by the protein. A non-limiting example of such a chromatin associated protein is the protein encoded by TOX. Some embodiments include measuring or detecting the presence or an amount of mRNA encoding one or more chromatin associated proteins.

In some embodiments, the chromatin associated protein can be a thymocyte selection associated high mobility group (HMG) box family member. In some embodiments, the target gene encodes a thymocyte selection associated HMG box family member. In some embodiments, the HMG box family member includes a HMG box DNA binding domain. In some embodiments, the HMG box family member includes multiple HMG box DNA binding domains. In some embodiments, the HMG box family member includes no more than one HMG box DNA binding domain. In some embodiments, the HMG box family member binds DNA in a sequence-independent manner. In some embodiments, the thymocyte selection associated HMG box family member is TOX. Some embodiments include measuring or detecting the presence or an amount of mRNA encoding one or more thymocyte selection associated HMG box family members.

In some embodiments, the target gene can encode a G-protein-coupled receptor (GPCR). In some embodiments, the GPCR is a receptor for a CC chemokine such as MCPCCL2, CCL4, CCL5, CCL17, CCL22, or CCL26. A non-limiting example of such a GPCR is the protein encoded by CCR4. Some embodiments include measuring or detecting the presence or an amount of mRNA encoding one or more GPCRs.

In some embodiments, the GPCR can be a C-C chemokine receptor type family member. In some embodiments, the target gene encodes a C-C chemokine receptor type family member. In some embodiments, the C-C chemokine receptor type family member is CCR4. Some embodiments include measuring or detecting the presence or an amount of mRNA encoding one or more C-C chemokine receptor type family members.

In some embodiments, the target gene can encode a gametocyte-specific family member. In some embodiments, the gametocyte-specific family member is GTSF1. Some embodiments include measuring or detecting the presence or an amount of mRNA encoding one or more gametocyte-specific family members.

In some embodiments, the gametocyte-specific family member can be a spermatogenesis protein. In some embodiments, the target gene encodes a spermatogenesis protein. In some embodiments, the spermatogenesis protein is expressed in testes. A non-limiting example of such a spermatogenesis protein is the protein encoded by GTSF1. Some embodiments include measuring or detecting the presence or an amount of mRNA encoding one or more spermatogenesis proteins.

In some embodiments, the target gene can encode an actin-binding protein. A non-limiting example of an actin-binding protein is the protein encoded by PLS3. Some embodiments include measuring or detecting the presence or an amount of mRNA encoding one or more actin-binding proteins.

In some embodiments, the actin-binding protein can be a plastin family member. In some embodiments, the target gene encodes a plastin family member. Some embodiments include measuring or detecting the presence or an amount of mRNA encoding one or more plastin family members. In some embodiments, the plastin family member is PLS3.

In some embodiments, the target gene can encode FYN binding protein, and is represented by “FYB.” In some embodiments, the target gene encodes lymphoid enhancer binding factor 1, and is represented by “LEF1.” In some embodiments, the target gene encodes IL2 inducible T-cell kinase, and is represented by “ITK.” In some embodiments, the target gene encodes interleukin 26, and is represented by “IL26.” In some embodiments, the target gene encodes signal transducer and activator of transcription 5A, and is represented by “STAT5A.” In some embodiments, the target gene encodes TRAF3 interacting protein 3, and is represented by “TRAF3IP3.” In some embodiments, the target gene encodes granulysin, and is represented by “GNLY.” In some embodiments, the target gene encodes dynamin 3, and is represented by “DNM3.” In some embodiments, the target gene encodes tumor necrosis factor superfamily member 11, and is represented by “TNFSF11.” In some embodiments, the target gene encodes thymocyte selection associated high mobility group box, and is represented by “TOX” In some embodiments, the target gene encodes C-C motif chemokine receptor 4, and is represented by “CCR4.” In some embodiments, the target gene encodes POU class 2 associating factor 1, and is represented by “POU2AF1.” In some embodiments, the target gene encodes gamctocyte specific factor 1, and is represented by “GTSF1.” In some embodiments, the target gene encodes plastin 3, and is represented by “PLS3.” In some embodiments, the target gene encodes matrix metallopeptidase 12, and is represented by “MMP12.” In some embodiments, the target gene encodes LCK proto-oncogene, Src family tyrosine kinase, and is represented by “LCK.” In some embodiments, the target gene encodes Neural precursor cell expressed, developmentally down-regulated, and is represented by “NEDD4L.” In some embodiments, the target gene encodes C-C motif chemokine ligand 27, and is represented by “CCL27.” In some embodiments, the target gene encodes chemokine (C-X-C motif) ligand 8, and is represented by “CXCL8.” CXCL8 may also be referred to as IL8. In some embodiments, the target gene encodes a chemokine such as the protein encoded by CXCL8. In some embodiments, the target gene encodes chemokine (C-X-C motif) ligand 9, and is represented by “CXCL9.” In some embodiments, the target gene encodes C-X-C motif chemokine 10, and is represented by “CXCL10.” In some embodiments, the target gene encodes tumor necrosis factor, and is represented by “TNF.” In some embodiments, the target gene encodes interleukin 13, and is represented by “IL13.” In some embodiments, the target gene encodes interleukin-4 receptor, and is represented by “IL4R.” In some embodiments, the target gene encodes C-C motif chemokine ligand 17, and is represented by “CCL17.” In some embodiments, the target gene encodes C-C motif chemokine ligand 26, and is represented by “CCL26.” In some embodiments, the target gene encodes interleukin 17A and is represented by “IL17A.” In some embodiments, the target gene encodes interleukin 23A, and is represented by “IL23A.” In some embodiments, the target gene encodes interleukin 22, and is represented by “IL22.” In some embodiments, the target gene encodes nitric oxide synthase 2, and is represented by “NOX2.”

In some embodiments, the at least one target gene comprises FYB, LEF1, GNLY, DMN3, ITK, IL26, STAT5, TRAF3IP3, TNFSF11, CCL27, CXCL8, CXCL9, CXCL10, and/or TNF, or a combination thereof. Some embodiments include measuring, obtaining, or measuring a gene expression level of FYB, LEF1, GNLY, DMN3, ITK, IL26, STAT5, TRAF3IP3, TNFSF11, CCL27, CXCL8, CXCL9, CXCL10, and/or TNF, or a combination thereof. In some embodiments, the at least one target gene comprises FYB. In some embodiments, the at least one target gene comprises GNLY. In some embodiments, the at least one target gene comprises ITK. In some embodiments, the at least one target gene comprises STAT5. In some embodiments, the at least one target gene comprises TRAF3IP3. In some embodiments, the at least one target gene comprises CXCL10. In some embodiments, the at least one target gene comprises CXCL8. In some embodiments, the at least one target gene comprises LEF1. In some embodiments, the at least one target gene comprises DMN3. In some embodiments, the at least one target gene comprises IL26. In some embodiments, the at least one target gene comprises TNFSF11. In some embodiments, the at least one target gene comprises CCL27. In some embodiments, the at least one target gene comprises CXCL9. In some embodiments, the at least one target gene comprises TNF. In some embodiments, the at least one target gene comprises TNF. In some embodiments, the at least one target gene one, two, three, four, five, six, seven, eight, nine, ten, eleven, twelve, thirteen, or fourteen of FYB, LEF1, GNLY, DMN3, ITK, IL26, STAT5, TRAF3IP3, TNFSF11, CCL27, CXCL8, CXCL9, CXCL10, and/or TNF. In some embodiments, gene data from the at least one target gene can be inputted into the non-transitory computer readable media or system described above.

In some embodiments, additional target genes may be measured, detected, or otherwise assayed and included in gene data inputted into the non-transitory computer readable media and/or system described above, and can be used in a method to generate prediction data using one or more diagnostic models to generate output data indicating if the tissue sample includes a skin disease and/or distinguish between CTCL and non-cancerous skin conditions (e.g., psoriasis, eczema, atopic dermatitis, and/or contact dermatitis). For example, in some embodiments the relate to a method of determining the presence of a non-cancerous skin condition such as eczema, psoriasis, atopic dermatitis, or contact dermatitis based on a presence or expression level of the target gene, and/or based on a mutation in the target gene. Some embodiments relate to a method of identifying a subject with the skin condition (e.g., eczema, psoriasis, atopic dermatitis, or contact dermatitis) based on a presence or expression level of the target gene, and/or based on a mutation in the target gene. Some embodiments include determining the presence of the skin condition (e.g., eczema, psoriasis, atopic dermatitis, or contact dermatitis) based on a presence or expression level of the target gene. Some embodiments include determining the presence of the skin condition (e.g., eczema, psoriasis, atopic dermatitis, or contact dermatitis) based on a mutation in the target gene. Some embodiments include the use of multiple target genes.

In some embodiments, the additional target gene may include one or more of IL13, IL4R, CCL17, CCL26, IL21A, IL22, NOS2, and/or IL17A, or a combination thereof. Some embodiments include measuring, obtaining, or measuring a gene expression level of IL13, IL4R, CCL17, CCL26, IL21A, IL22, NOS2, and/or IL17A, or a combination thereof. In some embodiments, the at least one target gene comprises IL13. In some embodiments, the at least one target gene comprises IL4R. In some embodiments, the at least one target gene comprises CCL17. In some embodiments, the at least one target gene comprises CCL26. In some embodiments, the at least one target gene comprises IL21A. In some embodiments, the at least one target gene comprises IL22. In some embodiments, the at least one target gene comprises NOS2. In some embodiments, the at least one target gene comprises IL17A. In some embodiments, the at least one target gene one, two, three, four, five, six, seven, or of IL13, IL4R, CCL17, CCL26, IL21A, IL22, NOS2, and/or IL17A. In some embodiments, gene data from the at least one target gene can be inputted into the non-transitory computer readable media or system described above. In some embodiments, one or more additional genes can be one or more of those disclosed in Table 1.

Measuring or determining expression levels of one or more target genes may be useful because some microRNAs are dysregulated in skin cancers such as CTCL. In some embodiments, one or more target genes are used to diagnose, identify, or determine the presence of a CTCL. In some embodiments, one or more target genes are used to rule out a skin cancer other than CTCL.

In some embodiments, the target gene encodes a microRNA (miRNA). In some embodiments, the miRNA is a small non-coding RNA. In some embodiments, the miRNA comprises or consists of 19-25 nucleotides. In some embodiments, the miRNA is from an intronic, intergenic, or antisense nucleic acid region. In some embodiments, the miRNA regulates post-transcriptional gene expression. Some embodiments described herein, include an RNA comprising a miRNA as described herein. Measuring or determining expression levels of one or more miRNAs may be useful because some miRNAs are dysregulated in skin cancers such as CTCL.

Examples of microRNAs include but are not limited to miR-21, miR-27b, miR-29b, miR-30c, miR-34a, miR-93, miR-141/200c, miR-142, miR-146, miR-148a, miR-152, miR-155, miR-181a/b, miR-186, miR-203, miR-205, miR-214, miR-221, miR-326, miR-486, miR-663b, and miR-711. In some embodiments, the microRNA comprises miR-21, miR-29b, miR-155, miR-186, miR-214, or miR-221. In some embodiments, the microRNA comprises miR-21. In some embodiments, the miR-21 is upregulated in a CTCL skin sample relative to a control. In some embodiments, the microRNA comprises miR-27b. In some embodiments, the miR-27b is upregulated in a CTCL skin sample relative to a control. In some embodiments, the microRNA comprises miR-29b. In some embodiments, the miR-29b is downregulated in a CTCL skin sample relative to a control. In some embodiments, the microRNA comprises miR-30c. In some embodiments, the miR-30c is upregulated in a CTCL skin sample relative to a control. In some embodiments, the microRNA comprises miR-34a. In some embodiments, the miR-34a is upregulated in a CTCL skin sample relative to a control. In some embodiments, the microRNA comprises miR-93. In some embodiments, the miR-93 is upregulated in a CTCL skin sample relative to a control. In some embodiments, the microRNA comprises miR-141/200c. In some embodiments, the miR-141/200c is upregulated in a CTCL skin sample relative to a control. In some embodiments, the microRNA comprises miR-142. In some embodiments, the miR-142 is upregulated in a CTCL skin sample relative to a control. In some embodiments, the microRNA comprises miR-146. In some embodiments, the miR-146 is upregulated in a CTCL skin sample relative to a control. In some embodiments, the microRNA comprises miR-148a. In some embodiments, the miR-148a is upregulated in a CTCL skin sample relative to a control. In some embodiments, the microRNA comprises miR-148b. In some embodiments, the miR-148b is upregulated in a CTCL skin sample relative to a control. In some embodiments, the microRNA comprises miR-152. In some embodiments, the miR-152 is upregulated in a CTCL skin sample relative to a control. In some embodiments, the microRNA comprises miR-155. In some embodiments, the miR-155 is upregulated in a CTCL skin sample relative to a control. In some embodiments, the microRNA comprises miR-181a/b. In some embodiments, the miR-181a/b is upregulated in a CTCL skin sample relative to a control. In some embodiments, the microRNA comprises miR-186. In some embodiments, the miR-186 is upregulated in a CTCL skin sample relative to a control. In some embodiments, the microRNA comprises miR-203. In some embodiments, the miR-203 is downregulated in a CTCL skin sample relative to a control. In some embodiments, the microRNA comprises miR-205. In some embodiments, the miR-205 is downregulated in a CTCL skin sample relative to a control. In some embodiments, the microRNA comprises miR-214. In some embodiments, the miR-214 is upregulated in a CTCL skin sample relative to a control. In some embodiments, the microRNA comprises miR-221. In some embodiments, the miR-221 is upregulated in a CTCL skin sample relative to a control. In some embodiments, the microRNA comprises miR-326. In some embodiments, the miR-326 is upregulated in a CTCL skin sample relative to a control. In some embodiments, the microRNA comprises miR-486. In some embodiments, the miR-486 is upregulated in a CTCL skin sample relative to a control. In some embodiments, the microRNA comprises miR-663b. In some embodiments, the miR-663b is upregulated in a CTCL skin sample relative to a control. In some embodiments, the microRNA comprises miR-711. In some embodiments, the miR-711 is upregulated in a CTCL skin sample relative to a control. Some embodiment include the use of multiple microRNAs as target genes. Some embodiment include the use of 1, 2, 3, 4, 5, 6, 7, 8, 9, 10, 11, 12, 13, 14, 15, or more microRNAs as target genes. Some embodiment include the use of a range of microRNAs as target genes, for example a range defined by any two of 1, 2, 3, 4, 5, 6, 7, 8, 9, 10, 11, 12, 13, 14, or 15.

In some embodiments, an amount of the miRNA is increased in CTCL relative to a non-CTCL control. In some embodiments, an amount of the miRNA is decreased in CTCL relative to a non-CTCL control. In some embodiments, the relative increase and/or decrease of miRNA can differ in tissue samples from a subject with CTCL as compared to tissue samples from a subject with a non-cancer skin condition (e.g., eczema, psoriasis, atopic dermatitis, and/or contact dermatitis).

In some embodiments, the miRNA is part of a cytokine or interleukin signaling pathway. For example, IL2 signaling may lead to upregulation of miR-155, miR-21, and miR-214, and/or downregulation of miR-29b. In some embodiments, STAT5 leads to miR-155 upregulation in response to IL2 signaling. In some embodiments, STAT3 leads to miR-21 upregulation in response to IL2 signaling. In some embodiments, CTCL comprises increased IL2 signaling and upregulated miR-155, miR-21, and miR-214, and downregulated miR-29b. MiR-21 may target PTEN. MiR-155 may target FOXO3A. MiR-214 may target PTEN, LHX6, Bcl2, and/or KIF12. MiR-29b may target MMP2, DNMT3, SP-1, and/or BRD4. Any of these microRNA targets may be dysregulated in a skin cancer such as CTCL, and thus may be used as target genes in the methods described herein.

In some aspects, CTCL may be diagnosed or determined, and/or benign inflammatory dermatoses (BID) and/or non-cancerous skin conditions (e.g., eczema, psoriasis, atopic dermatitis, and/or contact dermatitis) may be ruled out, based on upregulated expression of miR-326, miR-663b, miR-711, and/or miR-155 in CTCL compared to a control. In some aspects, CTCL may be diagnosed or determined, and/or BID may be ruled out, based on downregulated expression of miR-203 and/or miR-205 in CTCL compared to a control. In some embodiments, the miRNA expression is measured by microarray followed by PCR analysis. In some embodiments, these target genes are used to rule out a skin cancer other than CTCL.

In some aspects, CTCL may be diagnosed or determined, and/or benign inflammatory dermatoses (BID) and/or non-cancerous skin conditions (e.g., eczema, psoriasis, atopic dermatitis, and/or contact dermatitis) may be ruled out, based on upregulated expression of miR-155, miR-21, miR-142, miR-146, and/or miR-181a/b in CTCL compared to a control. In some aspects, CTCL may be diagnosed or determined, and/or BID may be ruled out, based on downregulated expression of miR-141/200c in CTCL compared to a control. In some embodiments, the microRNA expression is measured using a microarray. In some embodiments, these target genes are used to rule out a skin cancer other than CTCL.

In some aspects, Sezary syndrome (a type of CTCL) may be diagnosed or determined, or ruled out, based on upregulated expression of miR-21, miR-214, and/or miR-486 in Sézary syndrome compared to a control. In some embodiments, the microRNA expression is measured using a microarray. In some embodiments, these target genes are used to rule out a skin cancer or CTCL other than Sézary syndrome.

In some aspects, an aggressive form of CTCL may be diagnosed or determined based on upregulated expression of miR-181a, miR-93, and/or miR-34a in aggressive forms of CTCL compared to a control such as a non-cancerous skin sample or compared to a non-aggressive or benign form of CTCL. In some embodiments, the microRNA expression is measured with PCR.

In some aspects, CTCL may be diagnosed or determined, and/or benign inflammatory dermatoses (BID) and/or non-cancerous skin conditions (e.g., eczema, psoriasis, atopic dermatitis, and/or contact dermatitis) may be ruled out, based on upregulated or downregulated expression of miR-21. In some embodiments, the miR-21 expression is upregulated in a cancer such as bladder cancer. In some embodiments, the miR-21 expression is downregulated in a cancer such as PCNSL, glioblastoma, serosa-invasive gastric disorder, esophageal cancer, ovarian cancer, and/or NSCLC. In some embodiments, the miR-21 expression is measured in cerebrospinal fluid, ascites, urine, saliva, serum, and/or plasma.

In some embodiments, disclosed herein is a method of detecting the expression level of a gene from a gene classifier. In some instances, the method comprises detecting the expression level of FYN binding protein (FYB), IL2 inducible T-cell kinase (ITK), interleukin 26 (IL26), signal transducer and activator of transcription 5A (STAT5A), TRAF3 interacting protein 3 (TRAF3IP3), granulysin (GNLY), dynamin 3 (DNM3), tumor necrosis factor superfamily member 11 (TNFSF11), or a combination thereof. In some instances, the method comprises (a) isolating nucleic acids from a skin sample obtained from the subject, wherein the skin sample comprises cells from the stratum corneum; and (b) detecting the expression levels of FYB, LEF1, GNLY, DMN3, ITK, IL26, STAT5, TRAF3IP3, TNFSF11, CCL27, CXCL8, CXCL9, CXCL10, and/or TNF, or a combination thereof, by contacting the isolated nucleic acids with a set of probes that recognizes FYB, LEF1, GNLY, DMN3, ITK, IL26, STAT5, TRAF3IP3, TNFSF11, CCL27, CXCL8, CXCL9, CXCL10, and/or TNF, or a combination thereof, and detects binding between FYB, LEF1, GNLY, DMN3, ITK, IL26, STAT5, TRAF3IP3, TNFSF11, CCL27, CXCL8, CXCL9, CXCL10, and/or TNF, or a combination thereof and the set of probes. In the methods described herein, a gene classifier may include any target gene or combination of target genes described herein, and may include target gene expression levels or target gene mutations. Methods that describe a gene classifier may be used with target genes described herein in place of the gene classifier. In some embodiments, gene data from the at least one target gene can be inputted into the non-transitory computer readable media or system described above. In some embodiments, one or more additional genes as disclosed in Table 1 can be detected and quantified and imputed into the non-transitory computer readable media or system described above.

In some instances, the method comprises detecting the expression levels of two or more, three or more, or four or more of genes from the gene classifier: FYB, LEF1, GNLY, DMN3, ITK, IL26, STAT5, TRAF3IP3, TNFSF11, CCL27, CXCL8, CXCL9, CXCL10, and/or TNF. In some cases, the method comprises detecting the expression levels of ITK, STAT5A, and TNFSF11. In some cases, the method comprises detecting the expression levels of ITK, IL26, STAT5A, and TNFSF11. In some cases, the method comprises detecting the expression levels of FYB, ITK, IL26, STAT5A, and TNFSF11. In some cases, the method comprises detecting the expression levels of FYB, ITK, IL26, STAT5A, TRAF3IP3, and TNFSF11. In some cases, the method comprises detecting the expression levels of FYB, ITK, IL26, STAT5A, TRAF3IP3, DNM3, and TNFSF11. In some cases, the method comprises detecting the expression levels of FYB, ITK, IL26, STAT5A, TRAF3IP3, GNLY, DNM3, and TNFSF11. In some embodiments, gene data from the at least one target gene can be inputted into the non-transitory computer readable media or system described above. In some embodiments, one or more additional genes as disclosed in Table I can be detected and quantified and imputed into the non-transitory computer readable media or system described above.

In some instances, the expression level is an elevated gene expression level. In some cases, the elevated gene expression level is compared to a gene expression level of an equivalent gene from a control sample. In some cases, the control sample is a normal skin sample. In some cases, the gene expression level of FYB, LEF1, GNLY, DMN3, ITK, IL26, STAT5, TRAF3IP3, TNFSF11, CCL27, CXCL8, CXCL9, CXCL10, and/or TNF, or a combination thereof is elevated.

In some embodiments, the target gene expression is elevated by at least 1-fold, 2-fold, 3-fold, 4-fold, 5-fold, 10-fold, 20-fold, 30-fold, 40-fold, 50-fold, 60-fold, 70-fold, 80-fold, 90-fold, 100-fold, 110-fold, 120-fold, 130-fold, 150-fold, 200-fold, 300-fold, 500-fold, or more. In some embodiments, the target gene expression is decreased by at least 1-fold, 2-fold, 3-fold, 4-fold, 5-fold, 10-fold, 20-fold, 30-fold, 40-fold, 50-fold, 60-fold, 70-fold, 80-fold, 90-fold, 100-fold, 110-fold, 120-fold, 130-fold, 150-fold, 200-fold, 300-fold, 500-fold, or more. In some cases, the down-regulated gene expression level is compared to a control. In some embodiments, the control is a gene expression level of an equivalent gene from a control sample. In some cases, the control sample is a normal skin sample.

In some cases, the gene expression level of FYB, LEF1, GNLY, DMN3, ITK, IL26, STAT5, TRAF3IP3, TNFSF11, CCL27, CXCL8, CXCL9, CXCL10, and/or TNF is elevated by at least 1-fold, 2-fold, 3-fold, 4-fold, 5-fold, 10-fold, 20-fold, 30-fold, 40-fold, 50-fold, 60-fold, 70-fold, 80-fold, 90-fold, 100-fold, 110-fold, 120-fold, 130-fold, 150-fold, 200-fold, 300-fold, 500-fold, or more. In some cases, the gene expression level of FYB, LEF1, GNLY, DMN3, ITK, IL26, STAT5, TRAF3IP3, TNFSF11, CCL27, CXCL8, CXCL9, CXCL10, and/or TNF is elevated by at least 10-fold. In some cases, the gene expression level of FYB, LEF1, GNLY, DMN3, ITK, IL26, STAT5, TRAF3IP3, TNFSF11, CCL27, CXCL8, CXCL9, CXCL10, and/or TNF is elevated by at least 20-fold. In some cases, the gene expression level of FYB, LEF1, GNLY, DMN3, ITK, IL26, STAT5, TRAF3IP3, TNFSF11, CCL27, CXCL8, CXCL9, CXCL10, and/or TNF is elevated by at least 30-fold. In some cases, the gene expression level of FYB, LEF1, GNLY, DMN3, ITK, IL26, STAT5, TRAF3IP3, TNFSF11, CCL27, CXCL8, CXCL9, CXCL10, and/or TNF is elevated by at least 40-fold. In some cases, the gene expression level of FYB, LEF1, GNLY, DMN3, ITK, IL26, STAT5, TRAF3IP3, TNFSF11, CCL27, CXCL8, CXCL9, CXCL10, and/or TNF is elevated by at least 50-fold. In some cases, the gene expression level of FYB, LEF1, GNLY, DMN3, ITK, IL26, STAT5, TRAF3IP3, TNFSF11, CCL27, CXCL8, CXCL9, CXCL10, and/or TNF is elevated by at least 80-fold. In some cases, the gene expression level of FYB, LEF1, GNLY, DMN3, ITK, IL26, STAT5, TRAF3IP3, TNFSF11, CCL27, CXCL8, CXCL9, CXCL10, and/or TNF is elevated by at least 100-fold. In some cases, the gene expression level of FYB, LEF1, GNLY, DMN3, ITK, IL26, STAT5, TRAF3IP3, TNFSF11, CCL27, CXCL8, CXCL9, CXCL10, and/or TNF is elevated by at least 130-fold. In some cases, the gene expression level of FYB, LEF1, GNLY, DMN3, ITK, IL26, STAT5, TRAF3IP3, TNFSF11, CCL27, CXCL8, CXCL9, CXCL10, and/or TNF is elevated by at least 150-fold. In some cases, the elevated gene expression level is compared to a gene expression level of an equivalent gene from a control sample. In some cases, the control sample is a normal skin sample. In some embodiments, gene data from the at least one target gene expression can be inputted into the non-transitory computer readable media or system described above.

In some cases, the gene expression level of FYB, LEF1, GNLY, DMN3, ITK, IL26, STAT5, TRAF3IP3, TNFSF11, CCL27, CXCL8, CXCL9, CXCL10, and/or TNF is elevated by at least 10%, 20%, 30%, 40%, 50%, 60%, 70%, 80%, 90%, 100%, 200%, 300%, 400%, 500%, or more. In some cases, the gene expression level of FYB FYB, LEF1, GNLY, DMN3, ITK, IL26, STAT5, TRAF3IP3, TNFSF11, CCL27, CXCL8, CXCL9, CXCL10, and/or TNF is elevated by at least 10%. In some cases, the gene expression level of FYB, LEF1, GNLY, DMN3, ITK, IL26, STAT5, TRAF3IP3, TNFSF11, CCL27, CXCL8, CXCL9, CXCL10, and/or TNF is elevated by at least 30%. In some cases, the gene expression level of FYB, LEF1, GNLY, DMN3, ITK, IL26, STAT5, TRAF3IP3, TNFSF11, CCL27, CXCL8, CXCL9, CXCL10, and/or TNF is elevated by at least 50%. In some cases, the gene expression level of FYB, LEF1, GNLY, DMN3, ITK, IL26, STAT5, TRAF3IP3, TNFSF11, CCL27, CXCL8, CXCL9, CXCL10, and/or TNF is elevated by at least 80%. In some cases, the gene expression level of FYB, LEF1, GNLY, DMN3, ITK, IL26, STAT5, TRAF3IP3, TNFSF11, CCL27, CXCL8, CXCL9, CXCL10, and/or TNF is elevated by at least 100%. In some cases, the gene expression level of FYB, LEF1, GNLY, DMN3, ITK, IL26, STAT5, TRAF3IP3, TNFSF11, CCL27, CXCL8, CXCL9, CXCL10, and/or TNF is elevated by at least 200%. In some cases, the gene expression level of FYB, LEF1, GNLY, DMN3, ITK, IL26, STAT5, TRAF3IP3, TNFSF11, CCL27, CXCL8, CXCL9, CXCL10, and/or TNF is elevated by at least 300%. In some cases, the gene expression level of FYB, LEF1, GNLY, DMN3, ITK, IL26, STAT5, TRAF3IP3, TNFSF11, CCL27, CXCL8, CXCL9, CXCL10, and/or TNF is elevated by at least 500%. In some cases, the elevated gene expression level is compared to a gene expression level of an equivalent gene from a control sample. In some cases, the control sample is a normal skin sample. In some embodiments, gene data from the at least one target gene expression can be inputted into the non-transitory computer readable media or system described above.

In some instances, the expression level is a down-regulated gene expression level. In some cases, the gene expression level of GNLY is down-regulated. In some cases, the down-regulated gene expression level is compared to a gene expression level of an equivalent gene from a control sample. In some cases, the control sample is a normal skin sample.

In some instances, the gene expression level of GNLY is down-regulated by at least 1-fold, 2-fold, 3-fold, 4-fold, 5-fold, 10-fold, 20-fold, 30-fold, 40-fold, 50-fold, 60-fold, 70-fold, 80-fold, 90-fold, 100-fold, 110-fold, 120-fold, 130-fold, 150-fold, 200-fold, 300-fold, 500-fold, or more. In some cases, the gene expression level of GNLY is down-regulated by at least 1-fold. In some cases, the gene expression level of GNLY is down-regulated by at least 5-fold. In some cases, the gene expression level of GNLY is down-regulated by at least 10-fold. In some cases, the gene expression level of GNLY is down-regulated by at least 20-fold. In some cases, the gene expression level of GNLY is down-regulated by at least 30-fold. In some cases, the gene expression level of GNLY is down-regulated by at least 40-fold. In some cases, the gene expression level of GNLY is down-regulated by at least 50-fold. In some cases, the gene expression level of GNLY is down-regulated by at least 100-fold.

In some instances, the gene expression level of GNLY is down-regulated by at least 10%, 20%, 30%, 40%, 50%, 60%, 70%, 80%, 90%, 100%, 200%, 300%, 400%, 500%, or more. In some cases, the gene expression level of GNLY is down-regulated by at least 10%. In some cases, the gene expression level of GNLY is down-regulated by at least 20%. In some cases, the gene expression level of GNLY is down-regulated by at least 30%. In some cases, the gene expression level of GNLY is down-regulated by at least 50%. In some cases, the gene expression level of GNLY is down-regulated by at least 80%. In some cases, the gene expression level of GNLY is down-regulated by at least 100%.

In some embodiments, the set of probes recognizes at least one gene selected from FYB, LEF1, GNLY, DMN3, ITK, IL26, STAT5, TRAF3IP3, TNFSF11, CCL27, CXCL8, CXCL9, CXCL10, and/or TNF. In some cases, the set of probes recognizes ITK, STAT5A, and TNFSF11. In some cases, the set of probes recognizes ITK, IL26, STAT5A, and TNFSF11. In some cases, the set of probes recognizes FYB, ITK, IL26, STAT5A, and TNFSF11. In some cases, the set of probes recognizes FYB, ITK, IL26, STAT5A, TRAF3IP3, and TNFSF11. In some cases, the set of probes recognizes FYB, ITK, IL26, STAT5A, TRAF3IP3, DNM3, and TNFSF11. In some cases, the set of probes recognizes FYB, ITK, IL26, STAT5A, TRAF3IP3, GNLY, DNM3, and TNFSF11.

In some embodiments, the method further comprises detecting the expression levels of thymocyte selection associated high mobility group box (TOX); lymphoid enhancer binding factor 1 (LEF1); C-C motif chemokine receptor 4 (CCR4); POU class 2 associating factor 1 (POU2AF1); gametocyte specific factor 1 (GTSF1); plastin 3 (PLS3); matrix metallopeptidase 12 (MMP12); LCK proto-oncogene, Src family tyrosine kinase (LCK); neural precursor cell expressed, developmentally down-regulated (NEDD4L); or a combination thereof. In some cases, the detecting comprises contacting the isolated nucleic acids with an additional set of probes that recognizes TOX, LEF1, CCR4, POU2AF1, GTSF1, PLS3, MMP12, LCK, NEDD4L, or a combination thereof, and detects binding between TOX, LEF1, CCR4, POU2AF1, GTSF1, PLS3, MMP12, LCK, NEDD4L, or a combination thereof and the additional set of probes. In some embodiments, gene data from the gene expression of TOX, LEF1, CCR4, POU2AF1, GTSF1, PLS3, MMP12, LCK, and/or NEDD4L can be inputted into the non-transitory computer readable media or system described above.

In some cases, the additional set of probes recognizes one but no more than nine genes. In some cases, the additional set of probes recognizes 2, 3, 4, 5, 6, 7, 8, or 9 genes selected from TOX, LEF1, CCR4, POU2AF1, GTSF1, PLS3, MMP12, LCK, and NEDD4L.

In some cases, the expression level of one or more genes selected from TOX, LEF1, CCR4, POU2AF1, GTSF1, PLS3, MMP12, LCK, and NEDD4L is an elevated gene expression level. In such cases, the gene expression level is elevated by at least 1-fold, 2-fold, 3-fold, 4-fold, 5-fold, 10-fold, 20-fold, 30-fold, 40-fold, 50-fold, 60-fold, 70-fold, 80-fold, 90-fold, 100-fold, 110-fold, 120-fold, 130-fold, 150-fold, 200-fold, 300-fold, 500-fold, or more. In some instances, the gene expression level is elevated by at least 10%, 20%, 30%, 40%, 50%, 60%, 70%, 80%, 90%, 100%, 200%, 300%, 400%, 500%, or more. In some instances, the expression level is compared to a gene expression level of an equivalent gene from a control sample. In some instances, the control sample is a normal skin sample.

In additional cases, the expression level of one or more genes selected from TOX, LEF1, CCR4, POU2AF1, GTSF1, PLS3, MMP12, LCK, and NEDD4L is a down-regulated gene expression level. In such cases, the gene expression level is down-regulated by at least 1-fold, 2-fold, 3-fold, 4-fold, 5-fold, 10-fold, 20-fold, 30-fold, 40-fold, 50-fold, 60-fold, 70-fold, 80-fold, 90-fold, 100-fold, 110-fold, 120-fold, 130-fold, 150-fold, 200-fold, 300-fold, 500-fold, or more. In some instances, the gene expression level is down-regulated by at least 10%, 20%, 30%, 40%, 50%, 60%, 70%, 80%, 90%, 100%, 200%, 300%, 400%, 500%, or more. In some instances, the expression level is compared to a gene expression level of an equivalent gene from a control sample. In some instances, the control sample is a normal skin sample.

In some embodiments, the expression level of one or more additional genes from Table 1 can be determined and the expression level of the one or more additional genes can be elevated by at least 1-fold, 2-fold, 3-fold, 4-fold, 5-fold, 10-fold, 20-fold, 30-fold, 40-fold, 50-fold, 60-fold, 70-fold, 80-fold, 90-fold, 100-fold, 110-fold, 120-fold, 130-fold, 150-fold, 200-fold, 300-fold, 500-fold, or more, or elevated by at least 10%, 20%, 30%, 40%, 50%, 60%, 70%, 80%, 90%, 100%, 200%, 300%, 400%, 500%, or more. In some embodiments, the expression level of the one or more additional genes from Table 1 can be determined and expression of the one or more additional genes can be down-regulated by at least 1-fold, 2-fold, 3-fold, 4-fold, 5-fold, 10-fold, 20-fold, 30-fold, 40-fold, 50-fold, 60-fold, 70-fold, 80-fold, 90-fold, 100-fold, 110-fold, 120-fold, 130-fold, 150-fold, 200-fold, 300-fold, 500-fold, or more, or down-regulated elevated by at least 10%, 20%, 30%, 40%, 50%, 60%, 70%, 80%, 90%, 100%, 200%, 300%, 400%, 500%, or more.

In some embodiments, comparison of gene expression levels can be included in the gene data that can be inputted into one or more diagnostic models to generate prediction data using one or more diagnostic models to generate output data to indicate if the tissue sample includes a skin disease.

In some embodiments, a method described herein further comprises differentiating a skin cancer sample (e.g., a CTCL positive sample) from a non-cancer sample (e.g., normal skin, psoriasis, or atopic dermatitis). In some cases, the method has an improved specificity. In some instances, the specificity is at least or about 70%, 75%, 80%, 85%, 90%, or more than 95% when detecting the gene expression level of FYB, LEF1, GNLY, DMN3, ITK, IL26, STAT5, TRAF3IP3, TNFSF11, CCL27, CXCL8, CXCL9, CXCL10, TNF, or a combination thereof. In some embodiments, the specificity is at least or about 70%, 75%, 80%, 85%, 90%, or more than 95% when detecting the gene expression level of TOX, LEF1, CCR4, POU2AF1, GTSF1, PLS3, MMP12, LCK, NEDD4L, or a combination thereof. In some embodiments, the gene expression level of additional genes can also be detected, including the genes provided in Table 1.

In some cases, the method also has an improved sensitivity. In some embodiments, the sensitivity is at least or about 70%, 75%, 80%, 85%, 90%, or more than 95% when detecting the gene expression levels of FYB, LEF1, GNLY, DMN3, ITK, IL26, STAT5, TRAF3IP3, TNFSF11, CCL27, CXCL8, CXCL9, CXCL10, TNF, or a combination thereof. In some embodiments, the sensitivity is at least or about 70%, 75%, 80%, 85%, 90%, or more than 95% when detecting the gene expression levels of TOX, LEF1, CCR4, POU2AF1, GTSF1, PLS3, MMP12, LCK, NEDD4L, or a combination thereof. In some embodiments, the method also has a sensitivity of about 70%, 75%, 80%, 85%, 90%, or more than 95% when detecting the gene expression levels of additional genes as provided in Table 1.

In some embodiments, a system and method described herein comprises detecting gene expression levels from a first gene classifier and a second gene classifier in a subject in need thereof, comprising: (a) isolating nucleic acids from a skin sample obtained from the subject, wherein the skin sample comprises cells from the stratum corneum; (b) detecting the expression levels of one or more genes from the first gene classifier: FYB, LEF1, GNLY, DMN3, ITK, IL26, STAT5, TRAF3IP3, TNFSF11, CCL27, CXCL8, CXCL9, CXCL10, and/or TNF, by contacting the isolated nucleic acids with a set of probes that recognizes one or more genes from the first gene classifier, and detects binding between one or more genes from the first gene classifier and the set of probes; and (c) detecting the expression levels of one or more genes from the second gene classifier: TOX, LEF1, CCR4, POU2AF1, GTSF1, PLS3, MMP12, LCK, and NEDD4L, by contacting the isolated nucleic acids with an additional set of probes that recognizes one or more genes from the second gene classifier, and detects binding between one or more genes from the second gene classifier and the additional set of probes. In some embodiments, gene expression levels can be included in the gene data that can be inputted into one or more diagnostic models to generate prediction data using one or more diagnostic models to generate output data to indicate if the tissue sample includes a skin disease. In some embodiments, one or more genes as disclosed in Table I can be detected with the first gene classifier and/or the second gene classifier.

In some embodiments, a method described herein further comprises use of one or more additional targets to determine the presence of a skin cancer (e.g., CTCL). In some instances, the one or more additional targets include a target suitable for assessing CD4 to CD8 ratios, e.g., a target obtained from an immunohistochemistry analyses. In some instances, the one or more additional targets include CD4, CD7, CD8, and related CD markers such as CD45RA and CD45RO. In some instances, the one or more additional targets include a target suitable for assessing a loss of CD7 within a skin sample. In some instances, the one or more additional targets include a target suitable for assessing Th2 function (e.g., an increased expression of IL-4, IL-5, IL-10, or TGF-beta). In some instances, the one or more additional targets include a chemokine receptor family member such as CCR4 and CCR7. In some instances, the one or more additional targets include cutaneous lymphocyte-associated antigen (CLA). In some instances, the one or more additional targets include a miRNA or mutation associated with non-cutaneous lymphomas.

In some embodiments, a number of probes in the set of probes described above is at least or about 1, 2, 3, 5, 6, 7, 8, 9, 10, 11, 12, 13, 14, 15, 16, 17, 18, 19, 20, 25, 30, or more than 30 probes. In some embodiments, the number of probes in the set of probes is about 6 probes. In some embodiments, the number of probes in the set of probes is about 7 probes. In some embodiments, the number of probes in the set of probes is about 8 probes. In some embodiments, the number of probes in the set of probes is about 9 probes. In some embodiments, the number of probes in the set of probes is about 13 probes.

In some embodiments, the set of probes comprises one or more primer pairs. In some embodiments, a number of primer pairs is at least or about 1, 2, 3, 5, 6, 7, 8, 9, 10, 11, 12, 13, 14, 15, 16, 17, 18, 19, 20, 25, 30, or more than 30 primer pairs. In some embodiments, the number of primer pairs is about 6 primer pairs. In some embodiments, the number of primer pairs is about 7 primer pairs. In some embodiments, the number of primer pairs is about 13 primer pairs.

In some embodiments, one or more probes in the set of probes is labeled. In some embodiments, the one or more probe is labeled with a radioactive label, a fluorescent label, an enzyme, a chemiluminescent tag, a colorimetric tag, an affinity tag or other labels or tags that are known in the art.

Exemplary affinity tags include, but are not limited to, biotin, desthiobiotin, histidine, polyhistidine, myc, hemagglutinin (HA), FLAG, glutathione S transferase (GST), or derivatives thereof. In some embodiments, the affinity tag is recognized by avidin, streptavidin, nickel, or glutathione.

In some embodiments, the fluorescent label is a fluorophore, a fluorescent protein, a fluorescent peptide, quantum dots, a fluorescent dye, a fluorescent material, or variations or combinations thereof.

Exemplary fluorophores include, but are not limited to, Alexa-Fluor dyes (e.g., Alexa Fluor® 350, Alexa Fluor® 405, Alexa Fluor® 430, Alexa Fluor® 488, Alexa Fluor® 500, Alexa Fluor® 514, Alexa Fluor® 532, Alexa Fluor® 546, Alexa Fluor® 555, Alexa Fluor® 568, Alexa Fluor® 594, Alexa Fluor® 610, Alexa Fluor® 633, Alexa Fluor® 647, Alexa Fluor® 660, Alexa Fluor® 680, Alexa Fluor® 700, and Alexa Fluor® 750), APC, Cascade Blue, Cascade Yellow and R-phycoerythrin (PE), DyLight 405, DyLight 488, DyLight 550, DyLight 650, DyLight 680, DyLight 755, DyLight 800, FITC, Pacific Blue, PerCP, Rhodamine, and Texas Red, Cy5, Cy5.5, and/or Cy7.

Examples of fluorescent peptides include but are not limited to GFP (Green Fluorescent Protein) or derivatives of GFP (e.g., EBFP, EBFP2, Azurite, mKalama1, ECFP, Cerulean, CyPet, YFP, Citrine, Venus, and YPet.

Examples of fluorescent dyes include, but are not limited to, xanthenes (e.g., rhodamines, rhodols and fluoresceins, and their derivatives); bimanes; coumarins and their derivatives (e.g., umbelliferone and aminomethyl coumarins); aromatic amines (e.g., dansyl; squarate dyes); benzofurans; fluorescent cyanines; indocarbocyanines; carbazoles; dicyanomethylene pyranes; polymethine; oxabenzanthrane; xanthene; pyrylium; carbostyl; perylene; acridone; quinacridone; rubrene; anthracene; coronene; phenanthrecene; pyrene; butadiene; stilbene; porphyrin; pthalocyanine; lanthanide metal chelate complexes; rare-earth metal chelate complexes; and derivatives of such dyes. In some embodiments, the fluorescein dye is, but not limited to, 5-carboxyfluorescein, fluorescein-5-isothiocyanate, fluorescein-6-isothiocyanate and 6-carboxyfluorescein. In some embodiments, the rhodamine dye is, but not limited to, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, and rhodamine 101 sulfonyl chloride (sold under the tradename of TEXAS RED®). In some embodiments, the cyanine dye is Cy3, Cy3B, Cy3.5, Cy5, Cy5.5, Cy7, IRDYE680, Alexa Fluor 750, IRDye800CW, or ICG.

In some embodiments, the gene expression levels of FYB, LEF1, GNLY, DMN3, ITK, IL26, STAT5, TRAF3IP3, TNFSF11, CCL27, CXCL8, CXCL9, CXCL10, TNF, or a combination thereof is measured using PCR. Examples of PCR techniques include, but are not limited to quantitative PCR (qPCR), single cell PCR, PCR-RFLP, digital PCR (dPCR), droplet digital PCR (ddPCR), single marker qPCR, hot start PCR, and Nested PCR.

In some embodiments, the gene expression levels of TOX, LEF1, CCR4, POU2AF1, GTSF1, PLS3, MMP12, LCK, NEDD4L, or a combination thereof is measured using PCR. Examples of PCR techniques include, but are not limited to quantitative PCR (qPCR), single cell PCR, PCR-RFLP, digital PCR (dPCR), droplet digital PCR (ddPCR), single marker qPCR, hot start PCR, and Nested PCR.

In some embodiments, the expression levels are measured using qPCR. In some embodiments, the qPCR comprises use of fluorescent dyes or fluorescent probes. In some embodiments, the fluorescent dye is an intercalating dye. Examples of intercalating dyes include, but are not limited to, intercalating dyes include SYBR green I, SYBR green II, SYBR gold, ethidium bromide, methylene blue, Pyronin Y, DAPI, acridine orange, Blue View, or phycoerythrin. In some embodiments, the qPCR comprises use of more than one fluorescent probe. In some embodiments, the use of more than one fluorescent probes allows for multiplexing. For example, different non-classical variants are hybridized to different fluorescent probes and can be detected in a single qPCR reaction. Some embodiments include detecting or measuring an amount of binding between genes of interest and a set of probes, and includes detecting or measuring a fluorescent dye or a fluorescent probe.

Disclosed herein, in some embodiments, are methods of determining the presence of a skin cancer or non-Hodgkin's lymphoma such as a cutaneous T cell lymphoma (CTCL). Some embodiments include isolating nucleic acids from a skin sample obtained from a subject. Some embodiments include measuring, detecting, receiving, or using an expression level of a target gene. Some embodiments include detecting an expression level of a target gene in the skin sample. Some embodiments include measuring an expression level of a target gene in the skin sample. Some embodiments include receiving an expression level of a target gene in the skin sample. Some embodiments include using an expression level of a target gene in the skin sample. Some embodiments include measuring an expression level of a target gene in the skin sample. Some embodiments include measuring or detecting an expression level of the target gene.

Some embodiments include multiple target genes. For example, multiple target genes may be measured, detected, or used as gene data inputted into the diagnostic model described herein for generating prediction data and generating output data. Some embodiments include determining the presence of a skin cancer (e.g. CTCL) based on a presence or expression level of a first target gene and based on a mutation in a second target gene. Some embodiments include determining the presence of a skin cancer (e.g. CTCL) based on a presence or expression level of multiple target genes. Some embodiments include determining the presence of a skin cancer (e.g. CTCL) based on mutations in multiple target genes. Some embodiments include determining the presence of a skin cancer (e.g. CTCL) based on a presence or expression level of multiple target genes and based on mutations in multiple target genes.

Some embodiments include more than one target gene (e.g., at least one target gene). For example, the method may include measuring, detecting, receiving, or using expression levels of multiple target genes. Some embodiments include 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, 850, 900, 950, 1000, or more target genes. Some embodiments include 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, 850, 900, 950, 1000, or more target genes, or a range of target genes defined by any two of the aforementioned integers. For example, some embodiments include measuring or detecting an expression level of 17 target genes. Some embodiments include measuring or detecting an expression level of 8 target genes. Some embodiments include measuring or detecting an expression level of 1-10 target genes. Some embodiments include measuring or detecting an expression level of 1-100 target genes. Some embodiments include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, or at least 100 target genes. Some embodiments include no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 target genes.

In some embodiments, the nucleic acids comprise RNA. In some embodiments, the nucleic acids comprise mRNA. In some embodiments, measuring or detecting the expression level of the target gene comprises measuring or detecting an amount of RNA or mRNA encoded by a nucleic acid comprising the target gene. In some embodiments, measuring or detecting the expression level of the target gene comprises measuring or detecting an amount of mRNA encoded by a nucleic acid comprising the target gene. In some embodiments, using or receiving the expression level of the target gene comprises using or receiving information on an amount of RNA or mRNA encoded by a nucleic acid comprising the target gene.

Disclosed herein, in some embodiments, are target gene mutations. In some embodiments, the target gene comprises a target gene mutation. In some embodiments, the target gene mutation includes a hotspot somatic mutation (e.g. driver mutation). In some embodiments, the target gene mutation includes a significantly mutated gene. In some embodiments, the target gene mutation includes a hotspot somatic mutation from a significantly mutated gene. In some embodiments, the target gene comprises TP53. In some embodiments, the target gene comprises ZEB1. In some embodiments, the target gene comprises ARID1A. In some embodiments, the target gene comprises DNMT3A. In some embodiments, the target gene comprises CDKN2A. In some embodiments, the target gene comprises FAS. In some embodiments, the target gene comprises STAT5B. In some embodiments, the target gene comprises PRKCQ. In some embodiments, the target gene comprises RHOA. In some embodiments, the target gene comprises DNMT3A. In some embodiments, the target gene comprises PLCG1. In some embodiments, the target gene comprises NFKB2. In some embodiments, the target gene mutation comprises a mutation in any of TP53, ZEB1, ARID1A, DNMT3A, CDKN2A, FAS, STAT5B, PRKCQ, RHOA, DNMT3A, PLCG1, or NFKB2.

Some embodiments comprise a deletion mutation in one or more of TP53, ZEB1, ARID1A, DNMT3A, FAS, or CDKN2A. In some embodiments, the deletion mutation occurs in a subject with CTCL. Some embodiments comprise a deletion mutation in TP53. Some embodiments comprise a deletion mutation in ZEB1. Some embodiments comprise a deletion mutation in ARID1A. Some embodiments comprise a deletion mutation in DNMT3A. Some embodiments comprise a deletion mutation in FAS. Some embodiments comprise a deletion mutation in CDKN2A.

Some embodiments comprise a truncation. In some embodiments, the truncation occurs in a subject with CTCL. Some embodiments comprise a truncation of NFKB2. In some embodiments, the truncation is a C-terminal truncation. Some embodiments comprise a C-terminal truncation of NFKB2.

Some embodiments include a TP53 mutation. In some embodiments, the TP53 mutation comprises a Ser34* mutation. In some embodiments, the TP53 mutation comprises a Ser94* mutation. In some embodiments, the TP53 mutation comprises a Thr155Asn mutation. In some embodiments, the TP53 mutation comprises an Arg196*mutation. In some embodiments, the TP53 mutation comprises an Ala215Val mutation. In some embodiments, the TP53 mutation comprises an Ile254Thr mutation. In some embodiments, the TP53 mutation comprises an Arg273Pro mutation.

Some embodiments include a CD28 mutation. In some embodiments, the CD28 mutation comprises a Phe51Ile mutation. In some embodiments, the CD28 mutation comprises a Phe51Val mutation. In some embodiments, the CD28 mutation comprises a Gln77Pro mutation. In some embodiments, the CD28 mutation comprises a Lys81Asn mutation.

Some embodiments include a RhoA mutation. In some embodiments, the RhoA mutation comprises an Arg70Lys mutation. In some embodiments, the RhoA mutation comprises an Asn117Ile mutation.

Some embodiments include a DNMT3A mutation. In some embodiments, the DNMT3A mutation comprises a Pro233Leu mutation. In some embodiments, the DNMT3A mutation comprises a Tyr584* mutation. In some embodiments, the DNMT3A mutation comprises a Ser669Phe mutation. In some embodiments, the DNMT3A mutation comprises a Pro777Leu mutation.

Some embodiments include a FAS mutation. In some embodiments, the FAS mutation comprises a Ser212Cys mutation. In some embodiments, the FAS mutation comprises a Glu261Lys mutation. In some embodiments, the FAS mutation comprises an Asp265Glu mutation.

Some embodiments include a PLCG1 mutation. In some embodiments, the PLCG1 mutation comprises an Arg48Trp mutation. In some embodiments, the PLCG1 mutation comprises an Asp342Asn mutation. In some embodiments, the PLCG1 mutation comprises a Ser345Phe mutation. In some embodiments, the PLCG1 mutation comprises a Glu1163Lys mutation.

Some embodiments include detecting the presence at least one genotype of one more target genes. Some embodiments include detecting the presence at least one genotype of one more target genes known to be mutated in subjects with CTCL, in nucleic acids isolated from the skin sample of a subject suspected of having CTCL. In some embodiments, the nucleic acids comprise or consist of DNA. Some embodiments include determining whether the subject has CTCL based on the presence of the at least one genotype. Some embodiments include methods of determining the presence of a skin cancer such as a cutaneous T cell lymphoma (CTCL), using a target gene mutation as described herein. Some embodiments comprise detecting a mutational change in a target gene. Some embodiments include detecting a mutational change of a target gene.

Some embodiments relate to detecting expression levels of one or more target genes, and detecting a target gene mutation in one or more other target genes. Some embodiments relate to detecting expression levels of one or more target genes, and detecting a target gene mutation in one or more of the same target genes.

In some instances, the mutation is a missense substitution, a nonsense substitution (*), a coding silent substitution, deletion (del), an insertion (ins), or a frameshift (fs). In some instances, both expression level and mutational change provide information regarding the skin cancer in the subject. Information regarding the disease includes, but is not limited to, identification of a skin cancer, likelihood of treatment success for a skin cancer, identification of progression of a skin cancer, and identification of a skin cancer stage. In some instances, at least one of expression level and mutational change are compared to a control sample for identification of the skin cancer, determining likelihood of treatment success for the skin cancer, identification of progression of the skin cancer, or identification of the skin cancer stage. In some instances, the control sample is any sample that is used for making any one of these determinations. In some instances, the control sample is from a healthy individual. In some instances, the control is a sample from an individual with a known disease or disorder. In some instances, the control is from a database or reference. In some instances, the control is a normal sample from the same individual. In some instances, the normal sample is a sample that does not comprise skin cancer, or a sample that would test negative for skin cancer. In some instances, the normal sample is assayed at the same time or at a different time.

Disclosed herein, in some embodiments, are methods of determining the presence of a skin cancer such as a cutaneous T cell lymphoma (CTCL), comprising isolating nucleic acids from a skin sample obtained from a subject, and detecting an expression level of a target gene. Some embodiments include measuring or detecting an expression level of the target gene. Some embodiments include detecting an expression level of the target gene. Some embodiments include measuring an expression level of the target gene. Some embodiments include more than one target gene (e.g., at least one target gene). In some embodiments, measuring or detecting the expression level of the target gene comprises measuring or detecting an amount of RNA or mRNA encoded by a nucleic acid comprising the target gene. In some embodiments, the expression level of the target genes can be included as gene data inputted into non-transitory computer readable media or system to generate prediction data for generating output data to identify CTCL and/or distinguish CTCL from non-cancerous skin conditions.

Disclosed herein, in some embodiments, are methods of determining the presence of cutaneous T cell lymphoma (CTCL) in a skin sample. Some embodiments include identifying a subject suspected of having CTCL. Some embodiments include isolating nucleic acids from a skin sample obtained from the subject. In some embodiments, the skin sample is obtained by applying an adhesive patch to a skin region of the subject. In some embodiments, the adhesive patch is applied in a manner sufficient to adhere skin sample cells to the adhesive patch. In some embodiments, the skin sample is further obtained by removing the adhesive patch from the skin sample in a manner sufficient to retain the adhered skin sample cells to the adhesive patch. In some embodiments, the skin sample cells comprise cells from the stratum corneum. In some embodiments, the skin sample cells consist of cells from the stratum corneum. Some embodiments include isolating nucleic acids from a skin sample obtained from the subject by applying an adhesive patch to a skin region of the subject in a manner sufficient to adhere skin sample cells to the adhesive patch, and removing the adhesive patch from the skin sample in a manner sufficient to retain the adhered skin sample cells to the adhesive patch, wherein the skin sample cells comprise or consist of cells from the stratum corneum. Some embodiments include measuring or detecting an expression level of at least one target gene. In some embodiments, the at least one target gene is known to be upregulated or downregulated in subjects with CTCL. Some embodiments include contacting the isolated nucleic acids with a set of probes that recognize the target gene. Some embodiments include detecting binding between the at least one target gene and the set of probes.

Disclosed herein, in some embodiments, are methods of determining the presence of cutaneous T cell lymphoma (CTCL) in a skin sample, comprising: identifying a subject suspected of having CTCL; isolating nucleic acids from a skin sample obtained from the subject by applying an adhesive patch to a skin region of the subject in a manner sufficient to adhere skin sample cells to the adhesive patch, and removing the adhesive patch from the skin sample in a manner sufficient to retain the adhered skin sample cells to the adhesive patch, wherein the skin sample cells comprise cells from the stratum corneum; and measuring or detecting an expression level of at least one target gene known to be upregulated or downregulated in subjects with CTCL, by contacting the isolated nucleic acids with a set of probes that recognize the target gene, and detecting binding between the at least one target gene and the set of probes. In some embodiments, the expression level of the target genes can be included as gene data inputted into non-transitory computer readable media or system to generate prediction data for generating output data to identify CTCL and/or distinguish CTCL from non-cancerous skin conditions.

Some embodiments include determining whether the subject has CTCL based on the expression level of the at least one target gene. In some embodiments, the expression level is upregulated compared to a gene expression level of an equivalent gene from a control sample. In some embodiments, the expression level is downregulated compared to a gene expression level of an equivalent gene from a control sample. In some embodiments, the at least one target gene comprises a gene encoding an adapter protein. In some embodiments, the at least one target gene comprises a gene encoding a tyrosine kinase. In some embodiments, the at least one target gene comprises a gene encoding an interleukin. In some embodiments, the at least one target gene comprises a gene encoding a transcription factor. In some embodiments, the at least one target gene comprises a gene encoding a TNF receptor associated factor protein. In some embodiments, the at least one target gene comprises a gene encoding a TNF. In some embodiments, the at least one target gene comprises a gene encoding a saposin-like protein. In some embodiments, the at least one target gene comprises a gene encoding a GTP-binding protein. In some embodiments, the at least one target gene comprises a gene encoding a chromatin associated protein. In some embodiments, the at least one target gene comprises a gene encoding a G-protein-coupled receptor. In some embodiments, the at least one target gene comprises a gene encoding a transcriptional coactivator. In some embodiments, the at least one target gene comprises a gene encoding a spermatogenesis protein. In some embodiments, the at least one target gene comprises a gene encoding an actin-binding protein. In some embodiments, the at least one target gene comprises a gene encoding a matrix metalloproteinase. In some embodiments, the at least one target gene comprises a gene encoding a ubiquitin ligase. In some embodiments, the at least one target gene comprises a gene encoding modulator of cell death. In some embodiments, the at least one target gene comprises a gene encoding an antimicrobial. In some embodiments, the at least one target gene comprises a gene encoding a cytokine. In some embodiments, the at least one target gene comprises a gene encoding a DNA-binding protein. In some embodiments, the at least one target gene comprises a FYN-binding protein family member. In some embodiments, the at least one target gene comprises a TEC kinase family member. In some embodiments, the at least one target gene comprises a STAT. In some embodiments, the at least one target gene comprises a TRAF3 interacting protein. In some embodiments, the at least one target gene comprises a dynamin family member. In some embodiments, the at least one target gene comprises a TNF superfamily member. In some embodiments, the at least one target gene comprises a thymocyte selection associated high mobility group box family member. In some embodiments, the at least one target gene comprises a lymphoid enhancer binding factor family member. In some embodiments, the at least one target gene comprises a C-C chemokine receptor type family member. In some embodiments, the at least one target gene comprises an Oct binding factor family member. In some embodiments, the at least one target gene comprises a gametocyte-specific family member. In some embodiments, the at least one target gene comprises a plastin family member. In some embodiments, the at least one target gene comprises a lymphocyte-specific protein tyrosine kinase family member. In some embodiments, the at least one target gene comprises a member of the NEDD4 family of E3 HECT domain ubiquitin ligases. In some embodiments, the at least one target gene comprises a C-C motif chemokine ligand family member. In some embodiments, the at least one target gene comprises a chemokine. In some embodiments, the at least one target gene comprises a CXC chemokine. In some embodiments, the at least one target gene comprises a gene from Table 1.

In some embodiments, the at least one target gene comprises a gene encoding a saposin-like protein, a gene encoding a FYN-binding protein family member, a gene encoding a TEC kinase family member, a gene encoding a STAT, a gene encoding a TRAF3 interacting protein, a gene encoding a CXC chemokine family member, and/or a combination thereof. In some embodiments, the at least one target gene is upregulated.

Disclosed herein, in some embodiments, are methods for non-invasively identifying a cutaneous T cell lymphoma (CTCL) in a subject suspected of having the CTCL. In some embodiments, the method includes isolating nucleic acids from a skin sample adhered to an adhesive patch, the skin sample having been obtained from the subject suspected of having the CTCL. Some embodiments include contacting the isolated nucleic acids with a set of probes that recognize one or more genes of interest implicated in the CTCL. Some embodiments include detecting or measuring an amount of binding between the genes of interest and the set of probes. Some embodiments include comparing the amount of binding between the genes of interest and the set of probes to a control or threshold amount of binding. Some embodiments include identifying the subject as having the CTCL, or as not having the CTCL, based on the amount of binding between the genes of interest and the set of probes relative to the control or threshold of binding. Some embodiments include administering an effective amount of a therapeutic agent to the subject identified as having the CTCL.

Disclosed herein, in some embodiments, are methods for non-invasively identifying a cutaneous T cell lymphoma (CTCL) in a subject suspected of having NMSC, the method comprising: isolating nucleic acids from a skin sample adhered to an adhesive patch, the skin sample having been obtained from the subject suspected of having the CTCL; contacting the isolated nucleic acids with a set of probes that recognize one or more genes of interest implicated in CTCL; and detecting or measuring an amount of binding between the genes of interest and the set of probes.

Disclosed herein, in some embodiments, are methods for non-invasively identifying a cutaneous T cell lymphoma (CTCL). Some embodiments include identifying a subject suspected of having the CTCL. Some embodiments include applying an adhesive patch to the subject's skin in a manner sufficient to adhere a skin sample to the adhesive patch. Some embodiments include removing the adhesive patch from the subject's skin in a manner sufficient to retain the skin sample adhered to the adhesive patch. Some embodiments include obtaining expression levels of genes of interest implicated in CTCL, or determining an amount of binding between the genes of interest and a set of probes that recognize the genes of interest.

Disclosed herein, in some embodiments, are methods for non-invasively identifying a cutaneous T cell lymphoma (CTCL), comprising: identifying a subject suspected of having the CTCL; applying an adhesive patch to the subject's skin in a manner sufficient to adhere a skin sample to the adhesive patch; removing the adhesive patch from the subject's skin in a manner sufficient to retain the skin sample adhered to the adhesive patch; and obtaining expression levels of genes of interest implicated in CTCL, or determining an amount of binding between the genes of interest and a set of probes that recognize the genes of interest.

Disclosed herein, in some embodiments, are methods for non-invasively identifying cutaneous T cell lymphoma (CTCL) in a subject suspected of having CTCL. In some embodiments, the method includes isolating nucleic acids from a skin sample adhered to an adhesive patch. In some embodiments, the skin sample was obtained from the stratum corneum of the subject suspected of having CTCL. Some embodiments include contacting the isolated nucleic acids with a set of probes that recognize target genes; and detecting or measuring an amount of binding between the nucleic acids and the set of probes.

Disclosed herein, in some embodiments, are methods for non-invasively identifying cutaneous T cell lymphoma (CTCL) in a subject suspected of having CTCL, the method comprising: isolating nucleic acids from a skin sample adhered to an adhesive patch, the skin sample having been obtained from the stratum corneum of the subject suspected of having CTCL; contacting the isolated nucleic acids with a set of probes that recognize target genes; and detecting or measuring an amount of binding between the nucleic acids and the set of probes.

Disclosed herein, in some embodiments, are methods for non-invasively identifying cutaneous T cell lymphoma (CTCL). In some embodiments, the method includes identifying a subject suspected of having CTCL. Some embodiments include applying an adhesive patch to the subject's skin in a manner sufficient to adhere a skin sample to the adhesive patch. Some embodiments include removing the adhesive patch from the subject's skin in a manner sufficient to retain the skin sample adhered to the adhesive patch. Some embodiments include obtaining expression levels of target genes implicated in CTCL. Some embodiments include determining an amount of binding between the genes of interest and a set of probes that recognize the target genes.

Disclosed herein, in some embodiments, are methods for non-invasively identifying cutaneous T cell lymphoma (CTCL), comprising: identifying a subject suspected of having CTCL; applying an adhesive patch to the subject's skin in a manner sufficient to adhere a skin sample to the adhesive patch; removing the adhesive patch from the subject's skin in a manner sufficient to retain the skin sample adhered to the adhesive patch; and obtaining expression levels of target genes implicated in CTCL, or determining an amount of binding between the genes of interest and a set of probes that recognize the target genes.

Some embodiments of the methods described herein include detecting the presence at least one genotype of one more additional target genes known to be mutated in subjects with CTCL, in the nucleic acids or in a separate set of nucleic acids isolated from the skin sample. In some embodiments, the nucleic acids or the separate set of nucleic acids comprise DNA. In some embodiments, determining whether the subject has CTCL further comprises determining whether the subject has CTCL based on the presence of the at least one genotype.

Described herein, in some embodiments, are methods of detecting gene expression levels and mutational changes in a skin sample. In some embodiments, the method includes isolating nucleic acids from the skin sample. Some embodiments include measuring or detecting expression levels of one or more target genes. Some embodiments include detecting a mutational change of one or more other target genes. In some embodiments, the gene expression levels are detected by contacting the isolated nucleic acids with a set of probes, and detecting binding between the target genes and the set of probes. Some embodiments include contacting the isolated nucleic acids with a set of probes. Some embodiments include contacting detecting binding between the target genes and the set of probes. Some embodiments include detecting the gene expression levels by contacting the isolated nucleic acids with a set of probes, and detecting binding between the target genes and the set of probes.

Described herein, in some embodiments, are methods of detecting gene expression levels and mutational changes in a skin sample, comprising: isolating nucleic acids from the skin sample; and detecting the expression levels of one or more target genes; and a mutational change of one or more other target genes; wherein the gene expression levels are detected by contacting the isolated nucleic acids with a set of probes, and detecting binding between the target genes and the set of probes.

### Methods of Treatment

Disclosed herein, in some embodiments, are methods of treating a subject suspected of having skin cancer. Some embodiments include methods of treating a subject with a skin cancer. In some embodiments, the method includes identifying a subject suspected of having the skin cancer. Some embodiments include isolating nucleic acids from a skin sample of the subject. In some embodiments, the skin sample is obtained from the subject by applying an adhesive patch to a skin region of the subject. In some embodiments, the adhesive patch is applied in a manner sufficient to adhere skin sample cells. In some embodiments, the skin sample is obtained from the subject further by removing the adhesive patch from the skin sample. In some embodiments, the adhesive patch is removed in a manner sufficient to retain the adhered skin sample cells to the adhesive patch. In some embodiments, the skin sample cells comprise cells from the stratum corneum. In some embodiments, the skin sample cells consist of cells from the stratum corneum. Some embodiments include measuring or detecting an expression level of at least one target gene. The target gene may include any of the target genes s described herein. In some embodiments, the at least one target gene is known to be upregulated or downregulated in subjects with the skin cancer. In some embodiments, the at least one target gene is upregulated or downregulated in the subject. Some embodiments include contacting the isolated nucleic acids with a set of probes that recognize the target gene. Some embodiments include detecting binding between the at least one target gene and the set of probes. In some embodiments, the expression level is detected or measured by contacting the isolated nucleic acids with a set of probes that recognize the target gene, and detecting binding between the at least one target gene and the set of probes. Some embodiments include receiving the expression level of the at least one target gene, wherein the expression level was measured or detected using a method as described herein. Some embodiments include determining whether the subject has the skin cancer based on the expression level of the at least one target gene. Some embodiments include administering a skin cancer treatment to the subject. Some embodiments include administering the skin cancer treatment to the subject when the subject is determined to have the skin cancer based on the expression level of the at least one target gene. Some embodiments include not administering the skin cancer treatment to the subject if the subject is not determined to have cancer based on the expression level of the at least one target gene. Some embodiments include withholding the skin cancer treatment from the subject when the subject is not determined to have skin cancer based on the expression level of the at least one target gene. In some embodiments, the subject has the skin cancer. In some embodiments, the skin cancer is cutaneous T cell lymphoma (CTCL). In some embodiments, the skin cancer treatment is a CTCL treatment.

Disclosed herein, in some embodiments, are methods of treating a subject with cutaneous T cell lymphoma (CTCL), comprising: identifying a subject suspected of having CTCL; isolating nucleic acids from a skin sample obtained from the subject by applying an adhesive patch to a skin region of the subject in a manner sufficient to adhere skin sample cells to the adhesive patch, and removing the adhesive patch from the skin sample in a manner sufficient to retain the adhered skin sample cells to the adhesive patch, wherein the skin sample cells comprise cells from the stratum corneum; detecting an expression level of at least one target gene known to be upregulated or downregulated in subjects with CTCL, by contacting the isolated nucleic acids with a set of probes that recognize the target gene, and detecting binding between the at least one target gene and the set of probes; determining whether the subject has CTCL based on the expression level of the at least one target gene; and administering a CTCL treatment to the subject when the subject is determined to have CTCL based on the expression level of the at least one target gene, and not administering the CTCL treatment to the subject when the subject is not determined to have CTCL based on the expression level of the at least one target gene.

Disclosed herein, in some embodiments, are methods of treating a subject with cutaneous T cell lymphoma (CTCL). Some embodiments include identifying a subject suspected of having CTCL. Some embodiments include obtaining a skin sample the subject by applying the adhesive patch to the subject's skin in a manner sufficient to adhere the skin sample to the adhesive patch, and removing the adhesive patch from the subject's skin in a manner sufficient to retain the skin sample adhered to the adhesive patch. Some embodiments include isolating nucleic acids from the skin sample. Some embodiments include contacting the isolated nucleic acids with a set of probes that recognize one or more genes of interest implicated in CTCL. Some embodiments include detecting or measuring the amount of binding between the genes of interest and the set of probes. Some embodiments include identifying the subject as having CTCL, or as not having CTCL, based on the amount of binding between the genes of interest and the set of probes. Some embodiments include administering a treatment for the CTCL based on the determination of whether the subject has CTCL.

Disclosed herein, in some embodiments, are methods of treating a subject with cutaneous T cell lymphoma (CTCL), comprising: identifying a subject suspected of having CTCL; obtaining a skin sample the subject by applying the adhesive patch to the subject's skin in a manner sufficient to adhere the skin sample to the adhesive patch, and removing the adhesive patch from the subject's skin in a manner sufficient to retain the skin sample adhered to the adhesive patch; isolating nucleic acids from the skin sample; contacting the isolated nucleic acids with a set of probes that recognize one or more genes of interest implicated in CTCL; detecting or measuring the amount of binding between the genes of interest and the set of probes; identifying the subject as having CTCL, or as not having CTCL, based on the amount of binding between the genes of interest and the set of probes; and administering a treatment for the CTCL based on the determination of whether the subject has CTCL.

Disclosed herein, in some embodiments, are methods of treating a subject suspected of having cutaneous T cell lymphoma (CTCL). In some embodiments, the method includes isolating nucleic acids from a skin sample adhered to an adhesive patch. In some embodiments, the skin sample has been obtained from the subject's stratum corneum. Some embodiments include contacting the isolated nucleic acids with a set of probes that recognize target genes. Some embodiments include detecting or measuring an amount of binding between the nucleic acids and the set of probes. Some embodiments include administering to the subject a treatment for CTCL when the amount of binding between the nucleic acids and the set of probes is altered in the skin sample relative to a control or threshold amount of binding. Some embodiments include determining that the subject has CTCL when the amount of binding between the nucleic acids and the set of probes in the skin sample is altered relative to the control or threshold amount of binding. In some embodiments, the amount of binding between the nucleic acids and the set of probes in the skin sample is greater than the control or threshold amount of binding. In some embodiments, the amount of binding between the nucleic acids and the set of probes in the skin sample is less than the control or threshold amount of binding.

Disclosed herein, in some embodiments, are methods of treating a subject suspected of having cutaneous T cell lymphoma (CTCL), comprising: isolating nucleic acids from a skin sample adhered to an adhesive patch, the skin sample having been obtained from the subject's stratum corneum; contacting the isolated nucleic acids with a set of probes that recognize target genes; detecting or measuring an amount of binding between the nucleic acids and the set of probes; and administering to the subject a treatment for CTCL when the amount of binding between the nucleic acids and the set of probes is altered in the skin sample relative to a control or threshold amount of binding.

Described herein, in some embodiments, are methods of treatment that include administering a skin cancer treatment such as a cutaneous T cell lymphoma (CTCL) treatment to a subject. Some embodiments include administering a CTCL treatment to the subject based on a determination of whether the subject has CTCL. In some embodiments, the CTCL treatment comprises a pharmaceutical composition. In some embodiments, the CTCL treatment comprises a steroid treatment. In some embodiments, the CTCL treatment comprises interferon treatment. In some embodiments, the CTCL treatment comprises chemotherapy. In some embodiments, the CTCL treatment comprises phototherapy. In some embodiments, the CTCL treatment comprises radiation therapy. In some embodiments, the CTCL treatment comprises a surgery. In some embodiments, the CTCL treatment comprises a transplant. In some embodiments, the CTCL treatment comprises a bone marrow transplant. In some embodiments, the CTCL treatment comprises a steroid, interferon, chemotherapy, phototherapy, radiation therapy, or a bone marrow transplant.

In some embodiments, the CTCL treatment includes administration of bexarotene to the subject. In some embodiments, the bexarotene is in a gel. In some embodiments, the CTCL treatment includes administration of mechlorethamine to the subject. In some embodiments, the mechlorethamine is in a gel. In some embodiments, the CTCL treatment includes administration of a retinoid to the subject. In some embodiments, the CTCL treatment includes administration of a corticosteroid to the subject. In some embodiments, the CTCL treatment includes administration of imiquimod to the subject. In some embodiments, the CTCL treatment includes administration of local radiation to the subject. In some embodiments, the CTCL treatment includes administration of ultraviolet light to the subject. In some embodiments, the CTCL treatment includes administration of extracorporeal photopheresis to the subject. In some embodiments, the CTCL treatment includes administration of acitretin to the subject. In some embodiments, the CTCL treatment includes administration of bexarotene to the subject. In some embodiments, the CTCL treatment includes administration of interferon to the subject. In some embodiments, the CTCL treatment includes administration of methotrexate to the subject. In some embodiments, the CTCL treatment includes administration of romidepsin to the subject. In some embodiments, the CTCL treatment includes administration of vorinostat to the subject.

Some embodiments include 1, 2, 3, 4, 5, 6, 7, 8, 9, 10, 11, 12, 13, 14, or 15, or more administrations of the skin cancer treatment. Some embodiments include a range defined by any two of 1, 2, 3, 4, 5, 6, 7, 8, 9, 10, 11, 12, 13, 14, or 15, administrations of the skin cancer treatment. Some embodiments include administration daily, weekly, biweekly, or monthly.

In some embodiments, the skin cancer treatment includes a pharmaceutical composition. In some embodiments, the pharmaceutical composition is sterile. In some embodiments, the pharmaceutical composition includes a pharmaceutically acceptable carrier. In some embodiments, the pharmaceutically acceptable carrier comprises water. In some embodiments, the pharmaceutically acceptable carrier comprises a buffer. In some embodiments, the pharmaceutically acceptable carrier comprises a saline solution. In some embodiments, the pharmaceutically acceptable carrier comprises water, a buffer, or a saline solution. In some embodiments, the composition comprises a liposome. In some embodiments, the pharmaceutically acceptable carrier comprises liposomes, lipids, nanoparticles, proteins, protein-antibody complexes, peptides, cellulose, nanogel, or a combination thereof.

In some embodiments, the skin cancer treatment results in prevention, inhibition, or reversion of the skin cancer in the subject. Some embodiments relate to use of a skin cancer treatment herein in the method of preventing, inhibiting, or reversing the skin cancer. Some embodiments relate to a method of preventing, inhibiting, or reversing a skin cancer such as cutaneous T cell lymphoma (CTCL) in a subject in need thereof. Some embodiments include administering a pharmaceutical composition to a subject with the skin cancer. In some embodiments, the administration prevents, inhibits, or reverses the skin cancer in the subject. In some embodiments, the pharmaceutical composition prevents, inhibits, or reverses the skin cancer in the subject.

Disclosed herein, in some embodiments, are methods of treating a subject suspected of having a non-cancerous skin condition, comprising: isolating nucleic acids from a skin sample adhered to an adhesive patch, the skin sample having been obtained from the subject's stratum corneum; contacting the isolated nucleic acids with a set of probes that recognize target genes; detecting or measuring an amount of binding between the nucleic acids and the set of probes; and administering to the subject a treatment for the non-cancerous skin condition when the amount of binding between the nucleic acids and the set of probes is altered in the skin sample relative to a control or threshold amount of binding. In some embodiments, the non-cancerous skin condition can be eczema, psoriasis, atopic dermatitis, and/or contact dermatitis.

Described herein, in some embodiments, are methods of treatment that include administering treatment for non-cancerous skin conditions described herein to a subject. Some embodiments include administering a non-cancerous skin condition treatment to the subject based on a determination of whether the subject has a non-cancerous skin condition. In some embodiments, the non-cancerous skin condition treatment comprises a pharmaceutical composition. In some embodiments, the non-cancerous skin condition treatment comprises a steroid treatment or corticosteroid treatment. In some embodiments, the non-cancerous skin condition treatment comprises interferon treatment. In some embodiments, the CTCL treatment comprises treatment with retinoids, vitamin D or its analogs, calcineurin inhibitors, and/or salicylic acid. In some embodiments, the non-cancerous skin condition treatment comprises phototherapy. In some embodiments, the non-cancerous skin condition treatment comprises cyclosporine, and/or methotrexate.

Some embodiments include 1, 2, 3, 4, 5, 6, 7, 8, 9, 10, 11, 12, 13, 14, or 15, or more administrations of the non-cancerous skin condition treatment. Some embodiments include a range defined by any two of 1, 2, 3, 4, 5, 6, 7, 8, 9, 10, 11, 12, 13, 14, or 15, administrations of the non-cancerous skin condition treatment. Some embodiments include administration daily, weekly, biweekly, or monthly.

In some embodiments, the non-cancerous skin condition treatment includes a pharmaceutical composition. In some embodiments, the pharmaceutical composition is sterile. In some embodiments, the pharmaceutical composition includes a pharmaceutically acceptable carrier. In some embodiments, the pharmaceutically acceptable carrier comprises water. In some embodiments, the pharmaceutically acceptable carrier comprises a buffer. In some embodiments, the pharmaceutically acceptable carrier comprises a saline solution. In some embodiments, the pharmaceutically acceptable carrier comprises water, a buffer, or a saline solution. In some embodiments, the composition comprises a liposome. In some embodiments, the pharmaceutically acceptable carrier comprises liposomes, lipids, nanoparticles, proteins, protein-antibody complexes, peptides, cellulose, nanogel, or a combination thereof.

In some embodiments, the non-cancerous skin condition treatment results in prevention, inhibition, or reversion of the non-cancerous skin condition in the subject. Some embodiments relate to use of a non-cancerous skin condition treatment herein in the method of preventing, inhibiting, or reversing the non-cancerous skin condition. Some embodiments relate to a method of preventing, inhibiting, or reversing a non-cancerous skin condition such as eczema, psoriasis, atopic dermatitis, and/or contact dermatitis in a subject in need thereof. Some embodiments include administering a pharmaceutical composition to a subject with the non-cancerous skin condition. In some embodiments, the administration prevents, inhibits, or reverses the non-cancerous skin condition in the subject.

Some embodiments include administering a skin cancer treatment. In some embodiments, administering comprises giving, applying or bringing the skin cancer treatment or non-cancerous skin condition treatment into contact with the subject. In some embodiments, administration is accomplished by any of a number of routes. In some embodiments, administration is accomplished by a topical, oral, subcutaneous, intramuscular, intraperitoneal, intravenous, intrathecal or intradermal route.

### Components of the Skin Collection Kit

In some embodiments, the adhesive patch from the sample collection kit described herein comprises a first collection area comprising an adhesive matrix and a second area extending from the periphery of the first collection area. The adhesive matrix is located on a skin facing surface of the first collection area. The second area functions as a tab, suitable for applying and removing the adhesive patch. The tab is sufficient in size so that while applying the adhesive patch to a skin surface, the applicant does not come in contact with the matrix material of the first collection area. In some embodiments, the adhesive patch does not contain a second area tab. In some instances, the adhesive patch is handled with gloves to reduce contamination of the adhesive matrix prior to use.

In some embodiments, the first collection area is a polyurethane carrier film. In some embodiments, the adhesive matrix is comprised of a synthetic rubber compound. In some embodiments, the adhesive matrix is a styrene-isoprene-styrene (SIS) linear block copolymer compound. In some instances, the adhesive patch does not comprise latex, silicone, or both. In some instances, the adhesive patch is manufactured by applying an adhesive material as a liquid-solvent mixture to the first collection area and subsequently removing the solvent. In some embodiments, the adhesive matrix is configured to adhere cells from the stratum corneum of a skin sample.

The matrix material is sufficiently sticky to adhere to a skin sample. The matrix material is not so sticky that is causes scarring or bleeding or is difficult to remove. In some embodiments, the matrix material is comprised of a transparent material. In some instances, the matrix material is biocompatible. In some instances, the matrix material does not leave residue on the surface of the skin after removal. In certain instances, the matrix material is not a skin irritant.

In some embodiments, the adhesive patch comprises a flexible material, enabling the patch to conform to the shape of the skin surface upon application. In some instances, at least the first collection area is flexible. In some instances, the tab is plastic. In an illustrative example, the adhesive patch does not contain latex, silicone, or both. In some embodiments, the adhesive patch is made of a transparent material, so that the skin sampling area of the subject is visible after application of the adhesive patch to the skin surface. The transparency ensures that the adhesive patch is applied on the desired area of skin comprising the skin area to be sampled. In some embodiments, the adhesive patch is between about 5 and about 100 mm in length. In some embodiments, the first collection area is between about 5 and about 40 mm in length. In some embodiments, the first collection area is between about 10 and about 20 mm in length. In some embodiments the length of the first collection area is configured to accommodate the area of the skin surface to be sampled, including, but not limited to, about 19 mm, about 20 mm, about 21 mm, about 22 mm, about 23 mm, about 24 mm, about 25 mm, about 30 mm, about 35 mm, about 40 mm, about 45 mm, about 50 mm, about 55 mm, about 60 mm, about 65 mm, about 70 mm, about 75 mm, about 80 mm, about 85 mm, about 90 mm, and about 100 mm. In some embodiments, the first collection area is elliptical.

In further embodiments, the adhesive patch of this invention is provided on a peelable release sheet in the adhesive skin sample collection kit. In some embodiments, the adhesive patch provided on the peelable release sheet is configured to be stable at temperatures between −80° C. and 30° C. for at least 6 months, at least 1 year, at least 2 years, at least 3 years, and at least 4 years. In some instances, the peelable release sheet is a panel of a tri-fold skin sample collector.

In some instances, nucleic acids are stable on adhesive patch or patches when stored for a period of time or at a particular temperature. In some instances, the period of time is at least or about 1 day, 2 days, 3 days, 4 days, 5 days, 6 days, 7 days, 2 weeks, 3 weeks, 4 weeks, or more than 4 weeks. In some instances, the period of time is about 7 days. In some instances, the period of time is about 10 days. In some instances, the temperature is at least or about −80° C., −70° C., −60° C., −50° C., −40° C., −20° C., −10° C., −4° C., 0° C., 5° C., 15° C., 18° C., 20° C., 25° C., 30° C., 35° C., 40° C., 45° C., 50° C., or more than 50° C. The nucleic acids on the adhesive patch or patches, in some embodiments, are stored for any period of time described herein and any particular temperature described herein. For example, the nucleic acids on the adhesive patch or patches are stored for at least or about 7 days at about 25° C., 7 days at about 30° C., 7 days at about 40° C., 7 days at about 50° C., 7 days at about 60° C., or 7 days at about 70° C. In some instances, the nucleic acids on the adhesive patch or patches are stored for at least or about 10 days at about −80° C.

The peelable release sheet, in certain embodiments, is configured to hold a plurality of adhesive patches, including, but not limited to, 12, 11, 10, 9, 8, 7, 6, 5, 4, 3, 2, 1, from about 2 to about 8, from about 2 to about 7, from about 2 to about 6, from about 2 to about 4, from about 3 to about 6, from about 3 to about 8, from about 4 to about 10, from about 4 to about 8, from about 4 to about 6, from about 4 to about 5, from about 6 to about 10, from about 6 to about 8, or from about 4 to about 8. In some instances, the peelable release sheet is configured to hold about 12 adhesive patches. In some instances, the peelable release sheet is configured to hold about 11 adhesive patches. In some instances, the peelable release sheet is configured to hold about 10 adhesive patches. In some instances, the peelable release sheet is configured to hold about 9 adhesive patches. In some instances, the peelable release sheet is configured to hold about 8 adhesive patches. In some instances, the peelable release sheet is configured to hold about 7 adhesive patches. In some instances, the peelable release sheet is configured to hold about 6 adhesive patches. In some instances, the peelable release sheet is configured to hold about 5 adhesive patches. In some instances, the peelable release sheet is configured to hold about 4 adhesive patches. In some instances, the peelable release sheet is configured to hold about 3 adhesive patches. In some instances, the peelable release sheet is configured to hold about 2 adhesive patches. In some instances, the peelable release sheet is configured to hold about 1 adhesive patch.

Provided herein, in certain embodiments, are methods and compositions for obtaining a sample using an adhesive patch, wherein the adhesive patch is applied to the skin and removed from the skin. After removing the used adhesive patch from the skin surface, the patch stripping method, in some instances, further comprise storing the used patch on a placement area sheet, where the patch remains until the skin sample is isolated or otherwise utilized. In some instances, the used patch is configured to be stored on the placement area sheet for at least 1 week at temperatures between −80° C. and 30° C. In some embodiments, the used patch is configured to be stored on the placement area sheet for at least 2 weeks, at least 3 weeks, at least 1 month, at least 2 months, at least 3 months, at least 4 months, at least 5 months, and at least 6 months at temperatures between −80° C. to 30° C.

In some instances, the placement area sheet comprises a removable liner, provided that prior to storing the used patch on the placement area sheet, the removable liner is removed. In some instances, the placement area sheet is configured to hold a plurality of adhesive patches, including, but not limited to, 12, 11, 10, 9, 8, 7, 6, 5, 4, 3, 2, 1, from about 2 to about 8, from about 2 to about 7, from about 2 to about 6, from about 2 to about 4, from about 3 to about 6, from about 3 to about 8, from about 4 to about 10, from about 4 to about 8, from about 4 to about 6, from about 4 to about 5, from about 6 to about 10, from about 6 to about 8, or from about 4 to about 8. In some instances, the placement area sheet is configured to hold about 12 adhesive patches. In some instances, the placement area sheet is configured to hold about 11 adhesive patches. In some instances, the placement area sheet is configured to hold about 10 adhesive patches. In some instances, the placement area sheet is configured to hold about 9 adhesive patches. In some instances, the placement area sheet is configured to hold about 8 adhesive patches. In some instances, the placement area sheet is configured to hold about 7 adhesive patches. In some instances, the placement area sheet is configured to hold about 6 adhesive patches. In some instances, the placement area sheet is configured to hold about 5 adhesive patches. In some instances, the placement area sheet is configured to hold about 4 adhesive patches. In some instances, the placement area sheet is configured to hold about 3 adhesive patches. In some instances, the placement area sheet is configured to hold about 2 adhesive patches. In some instances, the placement area sheet is configured to hold about 1 adhesive patch.

The used patch, in some instances, is stored so that the matrix containing, skin facing surface of the used patch is in contact with the placement area sheet. In some instances, the placement area sheet is a panel of the tri-fold skin sample collector. In some instances, the tri-fold skin sample collector further comprises a clear panel. In some instances, the tri-fold skin sample collector is labeled with a unique barcode that is assigned to a subject. In some instances, the tri-fold skin sample collector comprises an area for labeling subject information.

In an illustrative embodiment, the adhesive skin sample collection kit comprises the tri-fold skin sample collector comprising adhesive patches stored on a peelable release panel. In some instances, the tri-fold skin sample collector further comprises a placement area panel with a removable liner. In some instances, the patch stripping method involves removing an adhesive patch from the tri-fold skin sample collector peelable release panel, applying the adhesive patch to a skin sample, removing the used adhesive patch containing a skin sample and placing the used patch on the placement area sheet. In some instances, the placement area panel is a single placement area panel sheet. In some instances, the identity of the skin sample collected is indexed to the tri-fold skin sample collector or placement area panel sheet by using a barcode or printing patient information on the collector or panel sheet. In some instances, the indexed tri-fold skin sample collector or placement sheet is sent to a diagnostic lab for processing. In some instances, the used patch is configured to be stored on the placement panel for at least 1 week at temperatures between −80° C. and 25° C. In some embodiments, the used patch is configured to be stored on the placement area panel for at least 2 weeks, at least 3 weeks, at least 1 month, at least 2 months, at least 3 months, at least 4 months, at least 5 months, and at least 6 months at temperatures between −80° C. and 25° C. In some embodiments, the indexed tri-fold skin sample collector or placement sheet is sent to a diagnostic lab using UPS or FedEx or other delivery service.

In an exemplary embodiment, the patch stripping method further comprises preparing the skin sample prior to application of the adhesive patch. Preparation of the skin sample includes, but is not limited to, removing hairs on the skin surface, cleansing the skin surface and/or drying the skin surface. In some instances, the skin surface is cleansed with an antiseptic including, but not limited to, alcohols, quaternary ammonium compounds, peroxides, chlorhexidine, halogenated phenol derivatives and quinolone derivatives. In some instances, the alcohol is about 0 to about 20%, about 20 to about 40%, about 40 to about 60%, about 60 to about 80%, or about 80 to about 100% isopropyl alcohol. In some instances, the antiseptic is 70% isopropyl alcohol.

In some embodiments, the patch stripping method is used to collect a skin sample from the surfaces including, but not limited to, the face, head, neck, arm, chest, abdomen, back, leg, hand or foot. In some instances, the skin surface is not located on a mucous membrane. In some instances, the skin surface is not ulcerated or bleeding. In certain instances, the skin surface has not been previously biopsied. In certain instances, the skin surface is not located on the soles of the feet or palms.

The patch stripping method, devices, and systems described herein are useful for the collection of a skin sample from a skin lesion. A skin lesion is a part of the skin that has an appearance or growth different from the surrounding skin. For example, the skin lesion may be due to skin cancer, such as CTCL or a non-cancerous skin condition, such as eczema, psoriasis, atopic dermatitis, and/or contact dermatitis. In some instances, the skin lesion is pigmented. A pigmented lesion includes, but is not limited to, a mole, dark colored skin spot and a melanin containing skin area. In some embodiments, the skin lesion is from about 5 mm to about 16 mm in diameter. In some instances, the skin lesion is from about 5 mm to about 15 mm, from about 5 mm to about 14 mm, from about 5 mm to about 13 mm, from about 5 mm to about 12 mm, from about 5 mm to about 11 mm, from about 5 mm to about 10 mm, from about 5 mm to about 9 mm, from about 5 mm to about 8 mm, from about 5 mm to about 7 mm, from about 5 mm to about 6 mm, from about 6 mm to about 15 mm, from about 7 mm to about 15 mm, from about 8 mm to about 15 mm, from about 9 mm to about 15 mm, from about 10 mm to about 15 mm, from about 11 mm to about 15 mm, from about 12 mm to about 15 mm, from about 13 mm to about 15 mm, from about 14 mm to about 15 mm, from about 6 to about 14 mm, from about 7 to about 13 mm, from about 8 to about 12 mm and from about 9 to about 11 mm in diameter. In some embodiments, the skin lesion is from about 10 mm to about 20 mm, from about 20 mm to about 30 mm, from about 30 mm to about 40 mm, from about 40 mm to about 50 mm, from about 50 mm to about 60 mm, from about 60 mm to about 70 mm, from about 70 mm to about 80 mm, from about 80 mm to about 90 mm, and from about 90 mm to about 100 mm in diameter. In some instances, the diameter is the longest diameter of the skin lesion. In some instances, the diameter is the smallest diameter of the skin lesion.

The patch stripping method, devices, and systems described herein are useful for the collection of a skin sample from a normal or healthy region of skin. A normal or healthy region of skin is a part of the skin that has an appearance or growth similar or identical to the surrounding skin and does not present with symptoms or indications suggestive of cancer or non-cancerous skin condition. In some embodiments, the skin sample from normal or healthy region of the skin may be utilized as a control sample for gene expression and/or gene data for comparison with gene expression and/or gene data from the skin lesion.

The adhesive skin sample collection kit, in some embodiments, comprises at least one adhesive patch, a sample collector, and an instruction for use sheet. In an exemplary embodiment, the sample collector is a tri-fold skin sample collector comprising a peelable release panel comprising at least one adhesive patch, a placement area panel comprising a removable liner, and a clear panel. The tri-fold skin sample collector, in some instances, further comprises a barcode and/or an area for transcribing patient information. In some instances, the adhesive skin sample collection kit is configured to include a plurality of adhesive patches, including but not limited to 12, 11, 10, 9, 8, 7, 6, 5, 4, 3, 2, 1, from about 2 to about 8, from about 2 to about 7, from about 2 to about 6, from about 2 to about 4, from about 3 to about 6, from about 3 to about 8, from about 4 to about 10, from about 4 to about 8, from about 4 to about 6, from about 4 to about 5, from about 6 to about 10, from about 6 to about 8, or from about 4 to about 8. The instructions for use sheet provide the kit operator all of the necessary information for carrying out the patch stripping method. The instructions for use sheet preferably include diagrams to illustrate the patch stripping method.

In some instances, the adhesive skin sample collection kit provides all the necessary components for performing the patch stripping method. In some embodiments, the adhesive skin sample collection kit includes a lab requisition form for providing patient information. In some instances, the kit further comprises accessory components. Accessory components include, but are not limited to, a marker, a resealable plastic bag, gloves and a cleansing reagent. The cleansing reagent includes, but is not limited to, an antiseptic such as isopropyl alcohol. In some instances, the components of the skin sample collection kit are provided in a cardboard box.

In some embodiments, the kit includes a skin collection device. In some embodiments, the skin collection device includes a non-invasive skin collection device. In some embodiments, the skin collection device includes an adhesive patch as described herein. In some embodiments, the skin collection device includes a brush. In some embodiments, the skin collection device includes a swab. In some embodiments, the skin collection device includes a probe. In some embodiments, the skin collection device includes a medical applicator. In some embodiments, the skin collection device includes a scraper. In some embodiments, the skin collection device includes an invasive skin collection device such as a needle or scalpel. In some embodiments, the skin collection device includes a needle. In some embodiments, the skin collection device includes a microneedle. In some embodiments, the skin collection device includes a hook.

Disclosed herein, in some embodiments, are kits for determining the presence of cutaneous T cell lymphoma (CTCL) in a skin sample. In some embodiments, the kit includes an adhesive patch. In some embodiments, the adhesive patch comprises an adhesive matrix configured to adhere skin sample cells from the stratum corneum of a subject. Some embodiments include a nucleic acid isolation reagent. Some embodiments include a plurality of probes that recognize at least one target gene. In some embodiments, the at least one target gene is known to be upregulated or downregulated in subjects with CTCL. Disclosed herein, in some embodiments, are kits for determining the presence of cutaneous T cell lymphoma (CTCL) in a skin sample, comprising: an adhesive patch comprising an adhesive matrix configured to adhere skin sample cells from the stratum corneum of a subject; a nucleic acid isolation reagent; and a plurality of probes that recognize at least one target gene known to be upregulated or downregulated in subjects with CTCL. Disclosed herein, in some embodiments, are kits for determining the presence of eczema, psoriasis, atopic dermatitis, and/or contact dermatitis in a skin sample, comprising: an adhesive patch comprising an adhesive matrix configured to adhere skin sample cells from the stratum corneum of a subject; a nucleic acid isolation reagent; and a plurality of probes that recognize at least one target gene known to be upregulated or downregulated in subjects with CTCL.

Disclosed herein, in some embodiments, are kits for determining the presence of a non-cancerous skin condition in a skin sample. In some embodiments, the kit includes an adhesive patch. In some embodiments, the adhesive patch comprises an adhesive matrix configured to adhere skin sample cells from the stratum corneum of a subject. Some embodiments include a nucleic acid isolation reagent. Some embodiments include a plurality of probes that recognize at least one target gene. In some embodiments, the at least one target gene is known to be upregulated or downregulated in subjects with a non-cancerous skin condition. Disclosed herein, in some embodiments, are kits for determining the presence of non-cancerous skin condition in a skin sample, comprising: an adhesive patch comprising an adhesive matrix configured to adhere skin sample cells from the stratum corneum of a subject; a nucleic acid isolation reagent; and a plurality of probes that recognize at least one target gene known to be upregulated or downregulated in subjects with non-cancerous skin condition. Disclosed herein, in some embodiments, are kits for determining the presence of eczema, psoriasis, atopic dermatitis, and/or contact dermatitis in a skin sample, comprising: an adhesive patch comprising an adhesive matrix configured to adhere skin sample cells from the stratum corneum of a subject; a nucleic acid isolation reagent; and a plurality of probes that recognize at least one target gene known to be upregulated or downregulated in subjects with non-cancerous skin condition.

Examples of subjects include but are not limited to vertebrates, animals, mammals, dogs, cats, cattle, rodents, mice, rats, primates, monkeys, and humans. In some embodiments, the subject is a vertebrate. In some embodiments, the subject is an animal. In some embodiments, the subject is a mammal. In some embodiments, the subject is an animal, a mammal, a dog, a cat, cattle, a rodent, a mouse, a rat, a primate, or a monkey. In some embodiments, the subject is a human. In some embodiments, the subject is male. In some embodiments, the subject is female. In some embodiments, the subject has, or is suspected of having, CTCL. In some embodiments, the CTCL comprises mycosis fungoides. In some embodiments, the CTCL comprises Sezary syndrome. In some embodiments, the subject has, or is suspected of having, a non-cancerous skin condition. In some embodiments, the non-cancerous skin condition comprises eczema. In some embodiments, the non-cancerous skin condition comprises psoriasis. In some embodiments, the non-cancerous skin condition comprises atopic dermatitis. In some embodiments, the non-cancerous skin condition comprises contact dermatitis.

### Cellular Material and Sample Process

In some embodiments of the methods described herein, a skin sample is obtained from the subject by applying an adhesive patch to a skin region of the subject. In some embodiments, the skin sample is obtained using an adhesive patch. In some embodiments, the adhesive patch comprises tape. In some embodiments, the skin sample is not obtained with an adhesive patch. In some instances, the skin sample is obtained using a brush. In some instances, the skin sample is obtained using a swab, for example a cotton swab. In some cases, the skin sample is obtained using a probe. In some cases, the skin sample is obtained using a hook. In some instances, the skin sample is obtained using a medical applicator. In some instances, the skin sample is obtained by scraping a skin surface of the subject. In some cases, the skin sample is obtained through excision. In some instances, the skin sample is biopsied. In some embodiments, the skin sample is a biopsy. In some instances, the skin sample is obtained using one or more needles. For example, the needles may be microneedles. In some instances, the biopsy is a needle biopsy, or a microneedle biopsy. In some instances, the skin sample is obtained invasively. In some instances, the skin sample is obtained non-invasively.

In some embodiments, the skin sample comprises cells of the stratum corneum. In some embodiments, the skin sample consists of cells of the stratum corneum. In some embodiments, the skin sample does not include the basal layer of the skin. In some embodiments, the skin sample comprises or consists of a skin depth of 10 μm, 50 μm, 100 μm, 150 μm, 200 μm, 250 μm, 300 μm, 350 μm, 400 μm, 450 μm, 500 μm, or a range of skin depths defined by any two of the aforementioned skin depths. In some embodiments, the skin sample comprises or consists of a skin depth of 50-100 μm. In some embodiments, the skin sample comprises or consists of a skin depth of 100-200 μm. In some embodiments, the skin sample comprises or consists of a skin depth of 200-300 μm. In some embodiments, the skin sample comprises or consists of a skin depth of 300-400 μm. In some embodiments, the skin sample comprises or consists of a skin depth of 400-500 μm.

In some embodiments, the skin sample is no more than 10 μm thick. In some embodiments, the skin sample is no more than 50 μm thick. In some embodiments, the skin sample is no more than 100 μm thick. In some embodiments, the skin sample is no more than 150 μm thick. In some embodiments, the skin sample is no more than 200 μm thick. In some embodiments, the skin sample is no more than 250 μm thick. In some embodiments, the skin sample is no more than 300 μm thick. In some embodiments, the skin sample is no more than 350 μm thick. In some embodiments, the skin sample is no more than 400 μm thick. In some embodiments, the skin sample is no more than 450 μm thick. In some embodiments, the skin sample is no more than 500 μm thick.

In some embodiments, the skin sample is at least 10 μm thick. In some embodiments, the skin sample is at least 50 μm thick. In some embodiments, the skin sample is at least 100 μm thick. In some embodiments, the skin sample is at least 150 μm thick. In some embodiments, the skin sample is at least 200 μm thick. In some embodiments, the skin sample is at least 250 μm thick. In some embodiments, the skin sample is at least 300 μm thick. In some embodiments, the skin sample is at least 350 μm thick. In some embodiments, the skin sample is at least 400 μm thick. In some embodiments, the skin sample is at least 450 μm thick. In some embodiments, the skin sample is at least 500 μm thick.

In some embodiments, the adhesive patch removes a skin sample from the subject at a depth no greater than 10 μm. In some embodiments, the adhesive patch removes a skin sample from the subject at a depth no greater than 50 μm. In some embodiments, the adhesive patch removes a skin sample from the subject at a depth no greater than 100 μm. In some embodiments, the adhesive patch removes a skin sample from the subject at a depth no greater than 150 μm. In some embodiments, the adhesive patch removes a skin sample from the subject at a depth no greater than 200 μm. In some embodiments, the adhesive patch removes a skin sample from the subject at a depth no greater than 250 μm. In some embodiments, the adhesive patch removes a skin sample from the subject at a depth no greater than 300 μm. In some embodiments, the adhesive patch removes a skin sample from the subject at a depth no greater than 350 μm. In some embodiments, the adhesive patch removes a skin sample from the subject at a depth no greater than 400 μm. In some embodiments, the adhesive patch removes a skin sample from the subject at a depth no greater than 450 μm. In some embodiments, the adhesive patch removes a skin sample from the subject at a depth no greater than 500 μm.

In some embodiments, the adhesive patch removes 1, 2, 3, 4, or 5 layers of stratum corneum from a skin surface of the subject. In some embodiments, the adhesive patch removes a range of layers of stratum corneum from a skin surface of the subject, for example a range defined by any two of the following integers: 1, 2, 3, 4, or 5. In some embodiments, the adhesive patch removes 1-5 layers of stratum corneum from a skin surface of the subject. In some embodiments, the adhesive patch removes 2-3 layers of stratum corneum from a skin surface of the subject. In some embodiments, the adhesive patch removes 2-4 layers of stratum corneum from a skin surface of the subject. In some embodiments, the adhesive patch removes no more than the basal layer of a skin surface from the subject.

In some embodiments, the adhesive patch removes an area of skin that is wholly within a skin area having or suspected of having a skin condition (e.g., CTCL or a non-cancerous skin condition). In some embodiments, the adhesive patch removes an area of skin that includes both a region having or suspected of having a skin condition (e.g., CTCL or a non-cancerous skin condition) and a region of healthy skin. In some embodiments, when the adhesive patch covers both healthy skin and skin having or suspected of having a skin condition, the adhesive patch can be marked to indicate areas of suspected healthy skin and suspected skin condition.

The methods and devices provided herein, in certain embodiments, involve applying an adhesive or other similar patch to the skin in a manner so that an effective or sufficient amount of a tissue, such as a skin sample, adheres to the adhesive matrix of the adhesive patch. In some cases, the skin sample adhered to the adhesive matrix comprises or consists of cells from the stratum corneum of a subject. For example, the effective or sufficient amount of a skin sample is an amount that removably adheres to a material, such as the matrix or adhesive patch. The adhered skin sample, in certain embodiments, comprises cellular material including nucleic acids. In some instances, the nucleic acid is RNA or DNA. An effective amount of a skin sample contains an amount of cellular material sufficient for performing a diagnostic assay. In some instances, the diagnostic assay is performed using the cellular material isolated from the adhered skin sample on the used adhesive patch. In some instances, the diagnostic assay is performed on the cellular material adhered to the used adhesive patch. In some embodiments, an effect amount of a skin sample comprises an amount of RNA sufficient to perform a gene expression analysis. Sufficient amounts of RNA include, but not limited to, picogram, nanogram, and microgram quantities. In some embodiments, the RNA includes mRNA. In some embodiments, the RNA includes microRNAs. In some embodiments, the RNA includes mRNA and microRNAs. In some embodiments, an effect amount of a skin sample comprises an amount of DNA sufficient to perform a gene expression analysis. Sufficient amounts of DNA include, but not limited to, picogram, nanogram, and microgram quantities. In some embodiments, an effect amount of a skin sample comprises an amount of DNA and RNA sufficient to perform a gene expression analysis. Sufficient amounts of DNA and RNA include, but not limited to, picogram, nanogram, and microgram quantities of the DNA and RNA.

Some embodiments include collecting cells from the stratum corneum of a subject, for instance, by using an adhesive tape with an adhesive matrix to adhere the cells from the stratum corneum to the adhesive matrix. In some embodiments, the cells from the stratum corneum comprise T cells or components of T cells. In some embodiments, the cells from the stratum corneum comprise keratinocytes. In some embodiments, the skin sample does not comprise melanocytes. In some embodiments, a skin sample is obtained by applying a plurality of adhesive patches to a skin region of a subject in a manner sufficient to adhere skin sample cells to each of the adhesive patches, and removing each of the plurality of adhesive patches from the skin region in a manner sufficient to retain the adhered skin sample cells to each of the adhesive patches. In some embodiments, the skin region comprises a skin lesion.

In some instances, the nucleic acid is a RNA molecule or a fragmented RNA molecule (RNA fragments). In some instances, the RNA is a microRNA (miRNA), a pre-miRNA, a primary miRNA (pri-miRNA), a mRNA, a pre-mRNA, a viral RNA, a viroid RNA, a virusoid RNA, circular RNA (circRNA), a ribosomal RNA (rRNA), a transfer RNA (tRNA), a pre-tRNA, a long non-coding RNA (lncRNA), a small nuclear RNA (snRNA), a circulating RNA, a cell-free RNA, an exosomal RNA, a vector-expressed RNA, a RNA transcript, a synthetic RNA, or combinations thereof. In some instances, the RNA is mRNA. In some instances, the RNA is cell-free circulating RNA.

In some instances, the nucleic acid is DNA. DNA includes, but not limited to, genomic DNA, viral DNA, mitochondrial DNA, plasmid DNA, amplified DNA, circular DNA, circulating DNA, cell-free DNA, or exosomal DNA. In some instances, the DNA is single-stranded DNA (ssDNA), double-stranded DNA, denaturing double-stranded DNA, synthetic DNA, and combinations thereof. In some instances, the DNA is genomic DNA. In some instances, the DNA is cell-free circulating DNA.

In additional embodiments, the adhered skin sample comprises cellular material including nucleic acids such as RNA or DNA, in an amount that is at least about 1 picogram. In some embodiments, the amount of cellular material is no more than about 1 nanogram. In further or additional embodiments, the amount of cellular material is no more than about 1 microgram. In still further or additional embodiments, the amount of cellular material is no more than about 1 gram.

In further or additional embodiments, the amount of cellular material is from about 1 picogram to about 1 gram. In further or additional embodiments, the cellular material comprises an amount that is from about 50 microgram to about 1 gram, from about 100 picograms to about 500 micrograms, from about 500 picograms to about 100 micrograms, from about 750 picograms to about 1 microgram, from about 1 nanogram to about 750 nanograms, or from about 1 nanogram to about 500 nanograms.

In further or additional embodiments, the amount of cellular material, including nucleic acids such as RNA or DNA, comprises an amount that is from about 50 microgram to about 500 microgram, from about 100 microgram to about 450 microgram, from about 100 microgram to about 350 microgram, from about 100 microgram to about 300 microgram, from about 120 microgram to about 250 microgram, from about 150 microgram to about 200 microgram, from about 500 nanograms to about 5 nanograms, or from about 400 nanograms to about 10 nanograms, or from about 200 nanograms to about 15 nanograms, or from about 100 nanograms to about 20 nanograms, or from about 50 nanograms to about 10 nanograms, or from about 50 nanograms to about 25 nanograms.

In further or additional embodiments, the amount of cellular material, including nucleic acids such as RNA or DNA, is less than about 1 gram, is less than about 500 micrograms, is less than about 490 micrograms, is less than about 480 micrograms, is less than about 470 micrograms, is less than about 460 micrograms, is less than about 450 micrograms, is less than about 440 micrograms, is less than about 430 micrograms, is less than about 420 micrograms, is less than about 410 micrograms, is less than about 400 micrograms, is less than about 390 micrograms, is less than about 380 micrograms, is less than about 370 micrograms, is less than about 360 micrograms, is less than about 350 micrograms, is less than about 340 micrograms, is less than about 330 micrograms, is less than about 320 micrograms, is less than about 310 micrograms, is less than about 300 micrograms, is less than about 290 micrograms, is less than about 280 micrograms, is less than about 270 micrograms, is less than about 260 micrograms, is less than about 250 micrograms, is less than about 240 micrograms, is less than about 230 micrograms, is less than about 220 micrograms, is less than about 210 micrograms, is less than about 200 micrograms, is less than about 190 micrograms, is less than about 180 micrograms, is less than about 170 micrograms, is less than about 160 micrograms, is less than about 150 micrograms, is less than about 140 micrograms, is less than about 130 micrograms, is less than about 120 micrograms, is less than about 110 micrograms, is less than about 100 micrograms, is less than about 90 micrograms, is less than about 80 micrograms, is less than about 70 micrograms, is less than about 60 micrograms, is less than about 50 micrograms, is less than about 20 micrograms, is less than about 10 micrograms, is less than about 5 micrograms, is less than about 1 microgram, is less than about 750 nanograms, is less than about 500 nanograms, is less than about 250 nanograms, is less than about 150 nanograms, is less than about 100 nanograms, is less than about 50 nanograms, is less than about 25 nanograms, is less than about 15 nanograms, is less than about 1 nanogram, is less than about 750 picograms, is less than about 500 picograms, is less than about 250 picograms, is less than about 100 picograms, is less than about 50 picograms, is less than about 25 picograms, is less than about 15 picograms, or is less than about 1 picogram.

In some embodiments, isolated RNA from a collected skin sample is reverse transcribed into cDNA, for example for amplification by PCR to enrich for target genes. The expression levels of these target genes are quantified by quantitative PCR in a gene expression test. In some instances, in combination with quantitative PCR, a software program performed on a computer is utilized to quantify RNA isolated from the collected skin sample. In some instances, a software program or module is utilized to relate a quantity of RNA from a skin sample to a gene expression signature, wherein the gene expression signature is associated with a disease such as skin cancer. In some embodiments, a software program or module scores a sample based on gene expression levels. In some embodiments, the sample score is compared with a reference sample score to determine if there is a statistical significance between the gene expression signature and a disease.

In some instances, the layers of skin include epidermis, dermis, or hypodermis. The outer layer of epidermis is the stratum corneum layer, followed by stratum lucidum, stratum granulosum, stratum spinosum, and stratum basale. In some instances, the skin sample is obtained from the epidermis layer. In some cases, the skin sample is obtained from the stratum corneum layer. In some instances, the skin sample is obtained from the dermis.

In some instances, cells from the stratum corneum layer are obtained, which comprises keratinocytes. In some instances, cells from the stratum corneum layer comprise T cells or components of T cells. In some cases, melanocytes are not obtained from the skin sample.

Following extraction of nucleic acids from a biological sample, the nucleic acids, in some instances, are further purified. In some instances, the nucleic acids are RNA. In some instances, the nucleic acids are DNA. In some instances, the RNA is human RNA. In some instances, the DNA is human DNA. In some instances, the RNA is microbial RNA. In some instances, the DNA is microbial DNA. In some instances, human nucleic acids and microbial nucleic acids are purified from the same biological sample. In some instances, nucleic acids are purified using a column or resin based nucleic acid purification scheme. In some instances, this technique utilizes a support comprising a surface area for binding the nucleic acids. In some instances, the support is made of glass, silica, latex or a polymeric material. In some instances, the support comprises spherical beads.

Methods for isolating nucleic acids, in certain embodiments, comprise using spherical beads. In some instances, the beads comprise material for isolation of nucleic acids. Exemplary material for isolation of nucleic acids using beads include, but not limited to, glass, silica, latex, and a polymeric material. In some instances, the beads are magnetic. In some instances, the beads are silica coated. In some instances, the beads are silica-coated magnetic beads. In some instances, a diameter of the spherical bead is at least or about 0.5 μm, 1 μm, 1.5 μm, 2 μm, 2.5 μm, 3 μm, 3.5 μm, 4 μm, 4.5 μm, 5 μm, 5.5 μm, 6 μm, 6.5 μm, 7 μm, 7.5 μm, 8 μm, 8.5 μm, 9 μm, 9.5 μm, 10 μm, or more than 10 μm.

In some cases, a yield of the nucleic acids products obtained using methods described herein is about 500 picograms or higher, about 600 picograms or higher, about 1000 picograms or higher, about 2000 picograms or higher, about 3000 picograms or higher, about 4000 picograms or higher, about 5000 picograms or higher, about 6000 picograms or higher, about 7000 picograms or higher, about 8000 picograms or higher, about 9000 picograms or higher, about 10000 picograms or higher, about 20000 picograms or higher, about 30000 picograms or higher, about 40000 picograms or higher, about 50000 picograms or higher, about 60000 picograms or higher, about 70000 picograms or higher, about 80000 picograms or higher, about 90000 picograms or higher, or about 100000 picograms or higher.

In some cases, a yield of the nucleic acids products obtained using methods described herein is about 100 picograms, 500 picograms, 600 picograms, 700 picograms, 800 picograms, 900 picograms, 1 nanogram, 5 nanograms, 10 nanograms, 15 nanograms, 20 nanograms, 21 nanograms, 22 nanograms, 23 nanograms, 24 nanograms, 25 nanograms, 26 nanograms, 27 nanograms, 28 nanograms, 29 nanograms, 30 nanograms, 35 nanograms, 40 nanograms, 50 nanograms, 60 nanograms, 70 nanograms, 80 nanograms, 90 nanograms, 100 nanograms, 500 nanograms, or higher.

In some cases, methods described herein provide less than less than 10%, less than 8%, less than 5%, less than 2%, less than 1%, or less than 0.5% product yield variations between samples.

In some cases, methods described herein provide a substantially homogenous population of a nucleic acid product.

In some cases, methods described herein provide less than 30%, less than 25%, less than 20%, less than 15%, less than 10%, less than 8%, less than 5%, less than 2%, less than 1%, or less than 0.5% contaminants.

In some instances, following extraction, nucleic acids are stored. In some instances, the nucleic acids are stored in water, Tris buffer, or Tris-EDTA buffer before subsequent analysis. In some instances, this storage is less than 8° C. In some instances, this storage is less than 4° C. In certain embodiments, this storage is less than 0° C. In some instances, this storage is less than −20° C. In certain embodiments, this storage is less than −70° C. In some instances, the nucleic acids are stored for about 1, 2, 3, 4, 5, 6, or 7 days. In some instances, the nucleic acids are stored for about 1, 2, 3, or 4 weeks. In some instances, the nucleic acids are stored for about 1, 2, 3, 4, 5, 6, 7, 8, 9, 10, 11, or 12 months.

In some instances, nucleic acids isolated using methods described herein are subjected to an amplification reaction following isolation and purification. In some instances, the nucleic acids to be amplified are RNA including, but not limited to, human RNA and human microbial RNA. In some instances, the nucleic acids to be amplified are DNA including, but not limited to, human DNA and human microbial DNA. Non-limiting amplification reactions include, but are not limited to, quantitative PCR (qPCR), self-sustained sequence replication, transcriptional amplification system, Q-Beta Replicase, rolling circle replication, or any other nucleic acid amplification known in the art. In some instances, the amplification reaction is PCR. In some instances, the amplification reaction is quantitative such as qPCR.

Provided herein are methods for detecting an expression level of one or more genes of interest from nucleic acids isolated from a biological sample. In some instances, the expression level is detected following an amplification reaction. In some instances, the nucleic acids are RNA. In some instances, the RNA is human RNA. In some instances, the RNA is microbial RNA. In some instances, the nucleic acids are DNA. In some instances, the DNA is human DNA. In some instances, the DNA is microbial DNA. In some instances, the expression level is determined using PCR. In some instances, the expression level is determined using qPCR. In some instances, the expression level is determined using a microarray. In some instances, the expression level is determined by sequencing.

Some embodiments include measuring a miRNA. In some embodiments, the measurement includes use of a stem-loop primer. Some embodiments include the use of poly-A tailing. Some embodiments include a pre-amplification of miRNAs.

Provided herein are methods and compositions for detecting a mutational change of one or more genes of interest from nucleic acids isolated from a biological sample. In some instances, the mutational change is detected following an amplification reaction. In some instances, the nucleic acids are RNA. In some instances, the nucleic acids are DNA. In some instances, the mutational change is detected using allele specific PCR. In some instances, the mutational change is detected using sequencing. In some instances, the sequencing is performed using the Sanger sequencing method. In some instances, the sequencing involves the use of chain terminating dideoxynucleotides. In some instances, the sequencing involves gel-electrophoresis. In some instances, the sequencing is performed using a next generation sequencing method. In some instances, sequencing includes, but not limited to, single-molecule real-time (SMRT) sequencing, Polony sequencing, sequencing by synthesis, sequencing by ligation, reversible terminator sequencing, proton detection sequencing, ion semiconductor sequencing, nanopore sequencing, electronic sequencing, pyrosequencing, Maxam-Gilbert sequencing, chain termination sequencing, +S sequencing, and sequencing by synthesis.

In some embodiments, the target gene mutation is detected using PCR. In some embodiments, the target gene mutation is detected using qPCR. In some embodiments, the target gene mutation is detected using sequencing. In some embodiments, the target gene mutation is detected using next generation sequencing. In some embodiments, the target gene mutation is detected using Sanger sequencing. In some embodiments, the target gene mutation is detected using an array. In some embodiments, the target gene mutation is detected using a mass spectrometry. In some embodiments, the target gene mutation is detected using a MassArray.

In some embodiments, the MassArray comprises mass spectrometry. In some embodiments, the MassArray includes DNA ionization, RNA separation, RNA detection, and/or an analysis of the detected RNAs. Some embodiments include a workflow including multiplex PCR, a mutant-specific extension protocol, and/or a MassArray analysis, followed by data analysis.

### Certain Terminologies

Unless defined otherwise, all technical and scientific terms used herein have the same meaning as is commonly understood by one of skill in the art to which the claimed subject matter belongs. It is to be understood that the detailed description are exemplary and explanatory only and are not restrictive of any subject matter claimed. In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

As used herein, the term “gene data(s)” refers to gene expression, gene mutation (e.g., nucleotide substitution, insertion, and/or deletion), gene duplication, and/or gene deletion within a tissue sample that can be measured or assessed by conventional means. Gene data can be stored on or inputted into the non-transitory computer readable media and system described herein. Gene data can be used by methods utilizing the non-transitory computer readable media and system to generate prediction data with one or more diagnostic models to indicate if and/or whether the tissue sample includes a skin disease.

FYN binding protein (FYB), also known as tyrosine-protein kinase FYN, Src-like kinase, tyrosine kinase P59fyn(T), or Src/Yes-related Novel, encodes a member of the protein-tyrosine kinase oncogene family. In some instances, FYB has National Center for Biotechnology Information (NCBI) Gene ID: 2534.

IL2 inducible T-cell kinase (ITK), also known as T-cell-specific kinase, tyrosine-protein kinase LYK, or IL2-inducible T-cell kinase, encodes an intracellular tyrosine kinase expressed in T-cells. In some instances, ITK has NCBI Gene ID: 3702.

Interleukin 26 (IL26) is also known as AK155 or protein AK155. In some instances, IL26 has NCBI Gene ID: 55801.

Signal transducer and activator of transcription 5A (STAT5A), also known as epididymis secretory sperm binding protein, encodes a member of the STAT family of transcription factors. In some instances, STAT5A has NCBI Gene ID: 6776.

TRAF3 interacting protein 3 (TRAF3IP3), (also known as TNF receptor associated factor 3, RING-type E3 ubiquitin transferase TRAF3, CD40 receptor associated factor 1, or T3JAM, encodes a member of the TNF receptor associated factor protein family. In some instances, TRAF3IP3 has NCBI Gene ID: 80342.

Granulysin (GNLY), also known as T-lymphocyte activation gene 519 or lymphokine LAG-2, encodes a member of the saposin-like protein family. In some instances, GNLY has NCBI Gene ID: 10578.

Dynamin 3 (DNM3), also known as T-dynamin, encodes a member of a family of guanosine triphosphate (GTP)-binding proteins. In some instances, DNM3 has Gene ID: 26052.

Tumor necrosis factor superfamily member 11 (TNFSF11), also known as osteoclast differentiation factor or osteoprotegerin ligand, encodes a member of TNF cytokine family of proteins. In some instances, TNFSF11 has NCBI Gene ID: 8600.

Thymocyte selection associated high mobility group box (TOX), also known as thymus high mobility group box protein TOX, encodes a protein containing a HMG box DNA binding domain. In some instances, TOX has NCBI Gene ID: 9760.

Lymphoid enhancer binding factor 1 (LEF1), also known as T cell-specific transcription factor 1-alpha or TCF7L3, encodes a transcription factor protein. In some instances, LEF1 has NCBI Gene ID: 51176.

C-C motif chemokine receptor 4 (CCR4), also known as CMKBR4, encodes a member of the G-protein-coupled receptor family. In some instances, CCR4 has NCBI Gene ID: 1233.

C-C chemokine ligand 27 (CCL27), also known as ALP, ILC, CTAK, CTACK, PESKY, ESKINE, and SCYA27, encodes a member of C-C chemokine ligands. In some instances, CCL27 has NCBI Gene ID: 10850.

C-X-C chemokine ligand 8 (CXCL8), also known as IL8, NAF, or GCP1, encodes a member of the CXC ligand. In some instances, CXCL8 has NCBI Gene ID: 3576.

C-X-C chemokine ligand 9 (CXCL9), also known as CMK, MIG, or SCYB9, encodes a member of the CXC ligand. In some instances, CXCL9 has NCBI Gene ID: 4283.

C-X-C chemokine ligand 10 (CXCL10), also known as IP10 or SCYB10, encodes a member of the CXC ligand. In some instances, CXCL10 has NCBI Gene ID: 3627.

POU class 2 associating factor 1 (POU2AF1), also known as B-cell-specific coactivator OBG-1, OCT-Binding factor 1, BOB-1, or OCA-B, is a protein coding gene. In some instances, POU2AF1 has NCBI Gene ID: 5450.

Gametocyte specific factor 1 (GTSF1), also known as family with sequence similarity 112, member B or FAM112B, encodes a protein involved in spermatogenesis. In some instances, GTSF1 has NCBI Gene ID: 121355.

Plastin 3 (PLS3), also known as T-Plastin, T fimbrin, or BMND18, encodes a family of the actin-binding proteins. In some instances, PLS3 has NCBI Gene ID: 5358.

Matrix metallopeptidase 12 (MMP12), also known as HME or macrophage elastase, encodes a member of the peptidase M10 family of matrix metalloproteinases. In some instances, MMP12 has NCBI Gene ID: 4321.

LCK proto-oncogene, Src family tyrosine kinase (LCK), also known as lymphocyte cell-specific protein-tyrosine kinase, T cell-specific protein-tyrosine kinase, or protein YT16, encodes a member of the Src family of protein tyrosine kinases. In some instances, LCK has NCBI Gene ID: 3932.

Neural precursor cell expressed, developmentally down-regulated (NEDD4L), also known as 4-like, E3 ubiquitin protein ligase, HECT-type E3 ubiquitin transferase NED4L, or NEDD4.2, encodes a member of the Nedd4 family of HECT domain E3 ubiquitin ligases. In some instances, NEDD4L has NCBI Gene ID: 23327.

Additional genes can include one or more of the genes disclosed in Table 1.

| | | | | | |
|---|---|---|---|---|---|
| AADACL2 | CXCL1 | IFN-γ | KRT15 | miR-186 | PTEN |
| AD000090.1 | CXCL11 | IGFL1 | KRT2 | miR-203 | R3GCC1L |
| AKT | CXCL5 | IKBKB | KRT33A | miR-205 | RAB5B |
| AKT1 | CXCLH | IL12B | KRT33B | miR-21 | RAC1 |
| AKT1S1 | CYCS | IL13 | KRT5 | miR-214 | RBM10 |
| AL139247.1 | CYP24A1 | IL13R | KRT6C | miR-221 | RHOA |
| ALDH2 | DDIT4 | IL13RA1 | KRT80 | miR-27b | RPL22 |
| ARID1A | DEFB4A | IL17 | KRT85 | miR-29b | RTP4 |
| ARID1B | DEFB4A, | IL17A | KRTAP1-1 | miR-30c | S100A7 |
| ARID2 | DEPTOR | IL17C | KRTAP11-1 | miR-326 | S100A8 |
| BRAF | DNMT1 | IL17F | KRTAP1-5 | miR-34a | S100A9 |
| C6orf218 | DNMT3A | IL17RA | KRTAP19-5 | miR-486 | SCAF11 |
| CAPZA2 | DNMT3B | IL17RC | KRTAP2-1 | miR-663b | SCD5 |
| CASC15 | DNMT3L | IL18R1 | KRTAP2-2 | miR-711 | SLC2A3 |
| CASP14 | EDN1 | IL1B | KRTAP3-1 | miR-93 | SMAD3 |
| CBFA2T3 | EEF2 | IL21 | KRTAP3-3 | MMP-1 | SOX11 |
| Cbl | EIF4A1 | IL21A | KRTAP8-1 | MT-ND3 | SPINK5 |
| CCL17 | EIF4E | IL22 | KRTAP9-4 | MT-ND4 | SPP1 |
| CCL18 | EIF4EBP1 | IL23 | LCE1F | MTOR | STAT3 |
| CCL19 | EIFRB | IL23A | LCE2C | MUC22 | STAT5B |
| CCL20 | EMSY | IL24 | LCE3A-E | NAMPT | STAT6 |
| CCL22 | EPAS1 | IL31 | LCE5A | NCOA1 | STEAP-AS2 |
| CCL26 | FABP5 | IL31RA | LCE6A | NF1 | STK4 |
| CCL4 | FAS | IL33 | LCN2 | NFKB1A | STRA6 |
| CCL5 | FAT1 | IL36RN | LINC00518 | NFKB2 | TAGLN |
| CCND1 | FBXW11 | IL37 | LINC02167 | NLRP10 | TERT |
| CCNE1 | FDCSP | IL4 | LINC02571 | NOS2 | TGase5 |
| CCT8 | FGFR3 | IL4R | LMNA | NOTCH1 | TIMP1 |
| CD4 | FH | IL6 | MAP2K1 | NOTCH2 | TIMP2 |
| CD45RA | FLG | IL8 | MAP2K2 | NOTCH3 | TNFRSF1A |
| CD45RO | FLG-AS1 | IRF7 | MAPK | NRAS | TNFRSF6B |
| CD68 | FMNL3 | ISR1 | MAPK1 | OAS3 | TNF-α |
| CD7 | FOS | IVL | MAPK3 | orRAS | TNXB |
| CD8 | FOXI3 | IVNS1ABP | MCPCCL2 | PBX2 | TP53 |
| CDK2 | FP236383.4 | JPH3 | MED16 | PIK3CA | TPM4 |
| CDKN2A | FP236383.5 | KIF1B | MGMT | PPP2R1A | TSBP1 |
| CIC | FSTL1 | KIT | miR- | PPP6C | TSLP |
| | | | 141/200c | | |
| CLDN1 | GBP1 | KLF4 | miR-142 | PRAME | TSLP/R |
| CLIP1 | GNAS | KPNA3 | miR-146 | PRKACA | UPP1 |
| CMPK2 | HCP5 | KRAS | miR-148a | PRKCQ | VEGFA |
| COL5A2 | HLA-B | KRT1 | miR-152 | PRR5L | WARS1 |
| CRCT1 | HLA-C | KRT10 | miR-155 | PSORS1C1 | ZEB1 |
| CTNNB1 | HRAS | KRT14 | miR-181a/b | PTCH1 | ZNF750 |

### EXAMPLES

These examples are provided for illustrative purposes only and not to limit the scope of the claims provided herein.

### Example 1

Epidermal skin samples (lesional and non-lesional samples) were collected with non-invasive adhesive patches. DNA and RNA were prepared by melting the adhesive patch with mineral oil to separate the cells from the patch. Cells were lysed with detergents and enzymes were added to stabilize the nucleic acids.

### Example 2

Epidermal skin samples (lesional and non-lesional samples) were collected with non-invasive adhesive patches. Total RNA was extracted from the skin samples on adhesive patches with a silica-coated magnetic bead-based extraction method. qRT-PCR was utilized for measurement of gene expression of both target genes and a house keeping gene. Quantification of the target expression utilized a Ct measurement of both the target and housekeeping genes measured in parallel in the qRT-PCR, and changes of the target gene expression in test samples were presented as ΔCt, where ΔCt=Ct.target−Ct.housekeeping. A smaller ΔCt value indicates a stronger (or increased) gene expression in the test samples, and vice versa. Changes of the target gene expression in lesional samples compared to control samples (either non-lesional or normal skin samples) were calculated from the ΔCt values of both lesional and control samples, and presented as ΔΔCt, where ΔΔCt=ΔCt.lesion−ΔCt.control. A smaller ΔΔCt indicates a smaller change of the target gene expression between the lesional and control samples, and these changes were presented as fold of changes (FC), calculated from the ΔΔCt value as FC=2^{-ΔΔCt}.

Similar expression patterns or T-cell receptor rearrangements in different lesions from the same subject are indicative of clonality which can also be indicative of the presence of CTCL or helpful in the diagnosis of CTCL.

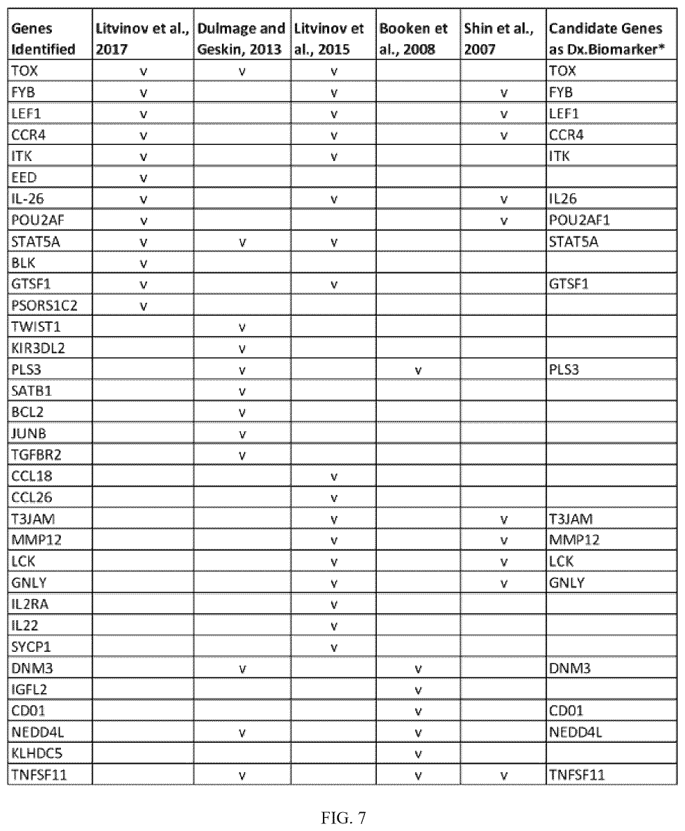

FIG. 7 illustrates exemplary gene expression biomarkers obtained from skin samples and tested for use as a diagnostic marker. The ‘V’ denotes genes displaying differential expression between CTCL tumor and normal skin samples, in FFPE tissues from biopsies, as reported in the respective study shown in the top row of the Figure.

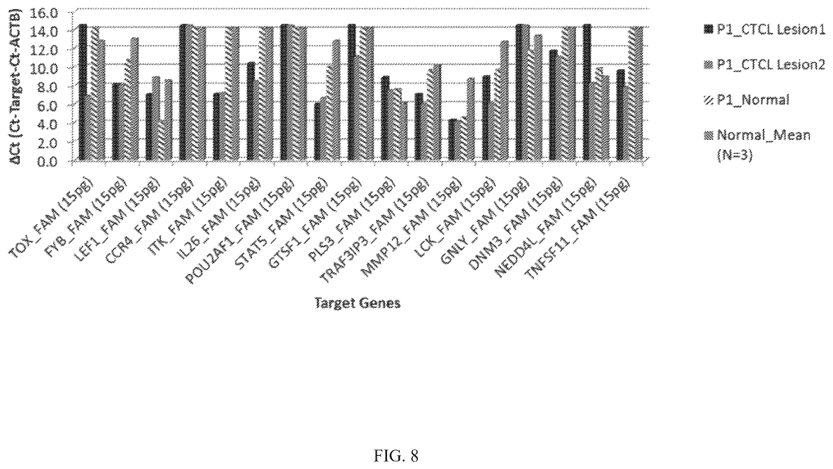

FIG. 8 shows the expression results of 17 exemplary genes tested in lesional, non-lesional, and healthy unaffected control skin samples obtained non-invasively via adhesive patches.

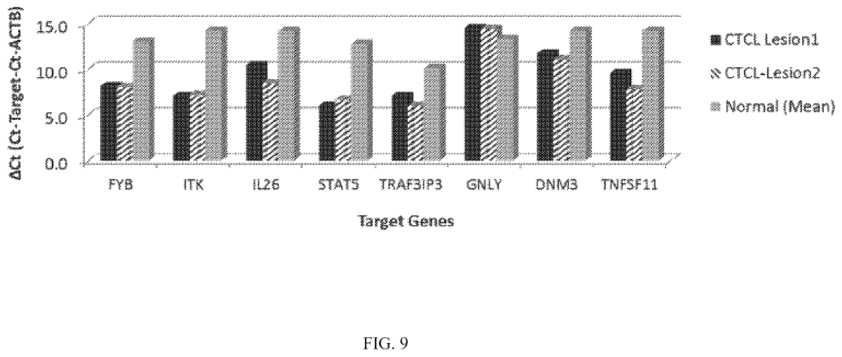

FIG. 9 shows the expression levels of exemplary target genes normalized to housekeeping genes analyzed in parallel (shown as ΔCt (=Ct.target−Ct.HouseKeeping).

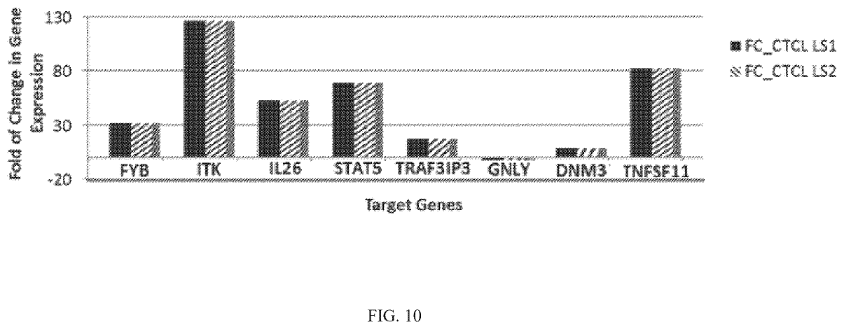

FIG. 10 shows fold change (FC) of the target genes from FIG. 9 in CTCL lesional skin samples compared to healthy unaffected controls (normal skin).

STAT5 shown in FIG. 8-FIG. 10 refers to STAT5A.

### Example 3

Additional skin samples, all collected with adhesive patches, were analyzed for gene expression changes by RT-qPCR following the procedures in Example 1. A total of 23 samples were included in the analysis. The samples included 12 CTCL samples and 11 normal skin samples, among which 6 were paired lesional and normal skin samples (i.e. each pair of sample came from one test subject or patient) and the rest were unpaired samples (lesional and normal skin samples from different test subjects).

The gene expression analysis included Ct values of target and housekeeping gene (ACTB) in qPCR from each sample; ΔCt values (=Ct_target−Ct_ACTB), for normalized gene expression levels in each sample; ΔΔCt values (=ΔCt_Lesion−ΔCt_NML), for changes in gene expression in lesional skins compared to normal skins in the paired samples (only the paired samples); P-values from statistical analysis of gene expression differences (based on ΔCt values) between the 2 groups of skin samples (lesional vs. normal/non-lesional); statistical analysis (with P-values). Five additional genes were included in the analysis performed in this example. Information relating to the genes in the gene expression analysis is included in FIGS. 6A-6B.

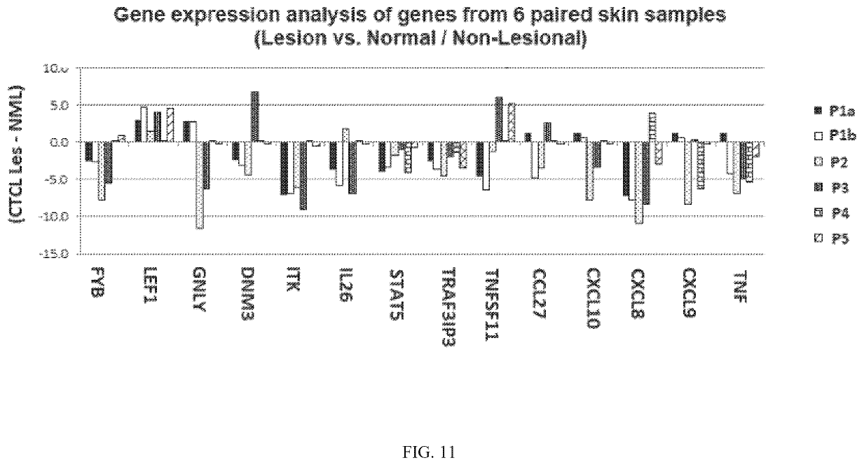

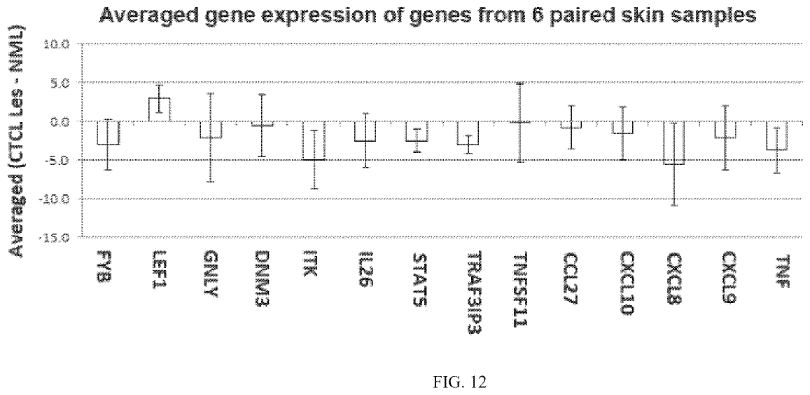

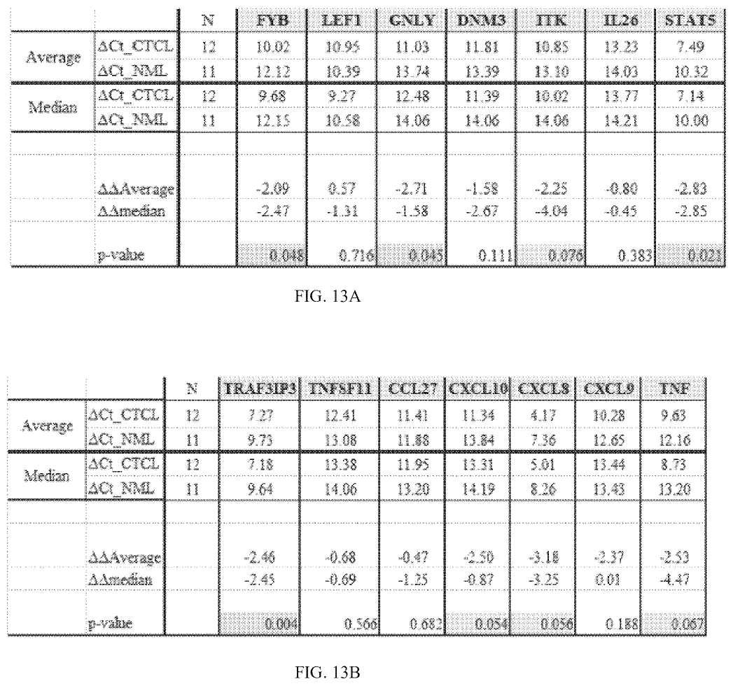

Gene expression data are shown in FIGS. 11-13B. A negative ΔΔCt value indicates an increased gene expression in lesional skin sample. The gene expression data show that 8 tested target genes had p-values below or close to 0.05, indicating that they may be used as target genes. The data indicated that the 8 genes may be used for a CTCL rule-out test. Of the 9 previously picked genes from Example 1, 4 had p-values below 0.05, and I had a p-value below 0.1 (p=0.076). Three of the 5 additional genes (compared to Example 1) showed increased gene expression matching increased protein levels reported in CTCL lesional skin samples.

### Example 4

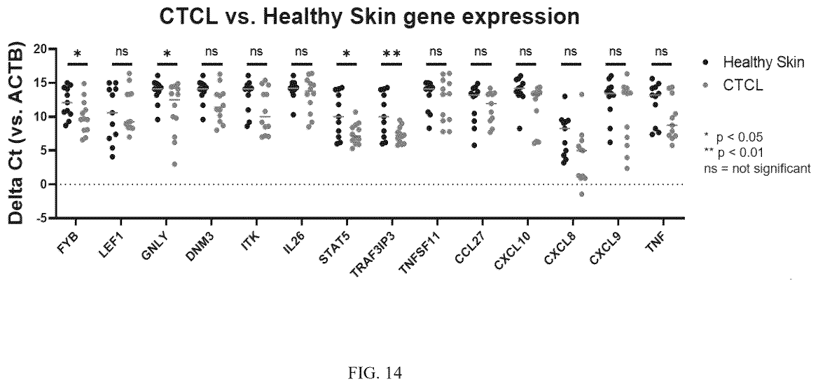

Skin samples were collected with adhesive patches and analyzed for changes in gene expression levels in CTCL lesion samples compared to paired normal (e.g., non-cancerous, healthy) skin samples. Expression levels of the following genes were determined: FYB, LEF1, GNLY, DNM3, ITK, IL26, STAT5, TRAF3IP3, CCL27, CXCL10, CXCL8, CXCL9, and TNF. The gene expression was inputted into an exemplary non-transitory, computer readable media as gene data and analyzed. CTCL samples showed decreased gene expression for the FYB, GNLY, STAT5A, and TRAF3IP3 genes as compared to normal skin, as shown in FIG. 14.

### Example 5

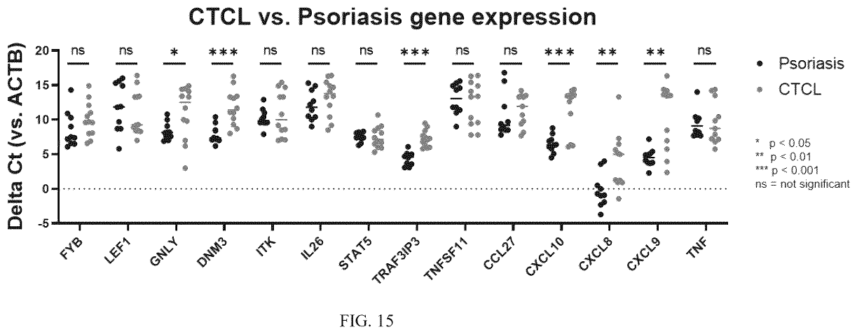

Skin samples were collected with adhesive patches and analyzed for changes in gene expression levels in CTCL lesion samples compared to paired psoriasis (e.g., non-cancerous) lesion samples. Expression levels of the following genes were determined: FYB, LEF1, GNLY, DNM3, ITK, IL26, STAT5, TRAF3IP3, CCL27, CXCL10, CXCL8, CXCL9, and TNF. The gene expression was inputted into an exemplary non-transitory, computer readable media as gene data and analyzed. CTCL samples showed increased gene expression for the GNLY, DNM3, TRAF3IP3, CXCL8, CXCL9, and CXCL10 genes as compared to psoriasis skin, as shown in FIG. 15.

### Example 6

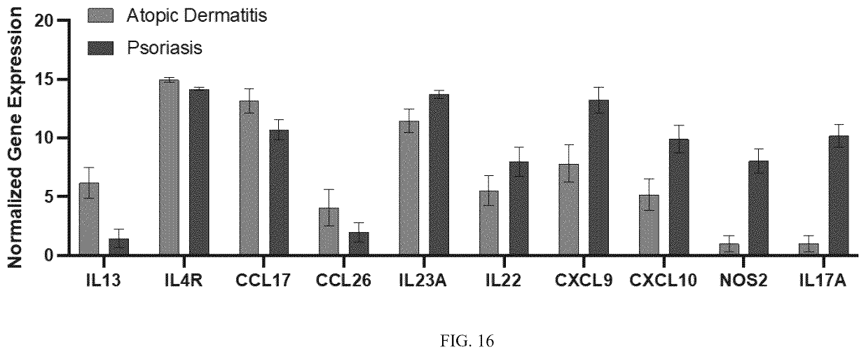

Skin samples were collected with adhesive patches and analyzed for changes in gene expression levels in atopic dermatitis lesion samples compared to paired psoriasis lesion samples. Expression levels of the following genes were determined: IL13, IL4R, CCL17, CCL26, IL23A, IL22, CXCL9, CXCL10, NOS2, and IL17A. The gene expression was inputted into an exemplary non-transitory, computer readable media as gene data and analyzed. Atopic dermatitis samples showed increased gene expression for the IL13, and CCL17 genes and decreased gene expression for the IL23a, IL22, CXCL9, CXCL10, NOS2, AND IL17A as compared to psoriasis skin, as shown in FIG. 16.

### Example 7

Skin samples will be collected with adhesive patches, and analyzed for changes in microRNA expression levels in CTCL lesion samples compared to paired normal skin samples. The expression levels of the following microRNAs will be analyzed to determine which are upregulated or downregulated compared to the control skin samples: miR-21, miR-27b, miR-29b, miR-30c, miR-34a, miR-93, miR-141/200c, miR-142, miR-146, miR-148a, miR-152, miR-155, miR-181a/b, miR-186, miR-203, miR-205, miR-214, miR-221, miR-326, miR-486, miR-663b, and miR-711. In some embodiments, the microRNA comprises miR-21, miR-29b, miR-155, miR-186, miR-214, and miR-221.

MicroRNA data will be grouped into with gene expression data from Example 2 to determine groupings of genes whose expression levels work exceptionally well for differentiating CTCL lesions from non-CTCL samples, compared to the individual gene expression levels.

### Example 8

Skin samples will be collected with adhesive patches, and analyzed for the presence and amount of in target gene mutations compared to paired normal skin samples. The mutational status of the following genes will be assessed: TP53, ZEB1, ARID1A, DNMT3A, CDKN2A, FAS, STAT5B, PRKCQ, RHOA, DNMT3A, PLCG1, and NFKB2.

Target gene mutation data will be assessed in combination with gene expression data from Examples 3 and/or 4 to determine groupings of target gene mutations and target gene expression levels that work exceptionally well for differentiating CTCL lesions from non-CTCL samples, compared to individual target gene mutations and expression levels.

### Example 9

A review of electronic and archived medical records of a private dermatology clinic was performed to identify all pigmented lesion assay (PLA) tests performed during the 9-year period between 2015 and 2024. The PLA was typically recommended when a lesion appeared clinically suspicious but was not deemed to require immediate biopsy. Patients with a positive PLA result were advised to return to clinic for shave biopsy, while those with a negative result were followed with clinical monitoring. For lesions that were biopsied, histopathologic diagnoses were categorized as benign, atypical (nevus with mild or moderate atypia), melanoma in situ (MIS, including nevi with severe atypia), or invasive melanoma (MM). For the purposes of this analysis, lesions diagnosed as MIS and those with severe atypia were grouped together due to their similar pathological features and management.

Study population characteristics and exclusion criteria are summarized in Table 2. In total, 5,758 PLA tests were performed over the 9-year period. Of these, 634 were positive for LINC00518, PRAME, or both. The remaining were either negative or could not be processed due to insufficient genomic material or sample contamination. Among the 634 PLA-positive lesions, 590 underwent biopsy or excision, with the corresponding pathology reviewed by an external dermatopathologist. The remaining 44 cases were either lost to follow-up or clinically monitored despite the recommendation for biopsy.

Of the 590 PLA-positive lesions with a corresponding biopsy, 344 were LINC00518+/PRAME− (17.7% (95% CI: 15.6-20.1%)), 110 were LINC00518−/PRAME+ (37.5% (95% CI: 31.4-44.1%)), and 136 were LINC00518+/PRAME+ (75.0% (95% CI: 68.1-80.8%)). Pathology results revealed 217 benign lesions, 168 atypical nevi, 194 MIS, and 11 invasive melanomas.

These PPVs were higher than previously reported values of 15%, 19%, and 67% respectively. Overall, 28.1 PLA tests were required to detect one melanoma. Importantly, use of the PLA led to monitoring—rather than biopsy—of over 5000 lesions, potentially reducing the number needed to biopsy per melanoma diagnosis to 21.4.5 Of the lesions that warranted a biopsy, 2.88 biopsies were needed to detect one melanoma.

Over time, the positivity rate increased, suggesting that clinician diagnostic skill improved with experience. There was a significant surge in positivity in 2021-22, however. During that period, which was the height of the COVID-19 pandemic, telemedicine was initiated. Rather than biopsy a suspicious lesion, which would have been possible in an in-person visit, clinicians sent patients a kit to perform PLA at home. Some lesions that were clearly atypical were sampled, possibly contributing to the sharp but transient rise in positivity rates.

In conclusion, the data show that dermoscopic evaluation prior to PLA testing improves the test's PPV. These findings support the use of dermoscopy and non-invasive gene expression testing to reduce unnecessary biopsies while maintaining high melanoma detection rates.

Furthermore, any term of degree such as, but not limited to, “substantially,” as used in the description and the appended claims, should be understood to include an exact, or a similar, but not exact configuration. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees.

Lastly, the terms “or” and “and/or,” as used herein, are to be interpreted as inclusive or meaning any one or any combination. Therefore, “A, B, or C” or “A, B, and/or C” mean any of the following: “A,” “B,” or “C”; “A and B”; “A and C”; “B and C”; “A, B and C.” An exception to this definition will occur only when a combination of elements, functions, steps or acts are in some way inherently mutually exclusive.

While the present disclosure has been described with reference to various implementations, it will be understood that these implementations are illustrative and that the scope of the present disclosure is not limited to them. Many variations, modifications, additions, and improvements are possible. More generally, implementations in accordance with the present disclosure have been described in the context of particular implementations. Functionality may be separated or combined differently in various implementations of the disclosure or described with different terminology. These and other variations, modifications, additions, and improvements may fall within the scope of the disclosure as defined in the claims that follow.

What is claimed is:

1. A non-transitory computer readable media storing computer-executable instructions that, when executed by at least one processor, cause a computing device to:
receive gene data, the gene data extracted from a tissue sample collected using an adhesive skin sample collector;
input the gene data into one or more diagnostic models;
generate prediction data using the one or more diagnostic models, the prediction data indicating if the tissue sample includes a skin disease based on the gene data; and
generate output data indicating if the tissue sample includes the skin disease.

2. The non-transitory computer readable media of claim 1, wherein the skin disease includes at least one of cutaneous T cell lymphoma (CTCL), eczema, psoriasis, atopic dermatitis, or contact dermatitis.

3. The non-transitory computer readable media of claim 1, wherein the gene data is obtained by isolating nucleic acids from the tissue sample, the tissue sample comprising cells from a stratum corneum.

4. The non-transitory computer readable media of claim 3, wherein the nucleic acids comprise mRNA.

5. The non-transitory computer readable media of claim 1, wherein the one or more diagnostic models is trained using historic gene data.

6. The non-transitory computer readable media of claim 3, wherein the tissue sample comprises cells from a subject having or suspected of having CTCL, eczema, psoriasis, atopic dermatitis, or contact dermatitis.

7. The non-transitory computer readable media of claim 1, wherein the gene data comprises expression level of one or more genes selected from a group consisting of FYB, LEF1, GNLY, DMN3, ITK, IL26, STAT5, TRAF3IP3, TNFSF11, CCL27, CXCL8, CXCL9, CXCL10, and TNF.

8. A method for detecting a skin condition, the method comprising:
receiving gene data, the gene data extracted from a tissue sample collected using an adhesive patch;
inputting the gene data into one or more diagnostic models;
generating prediction data using the one or more diagnostic models, the prediction data indicating if the tissue sample includes the skin condition based on the gene data; and
generating output data indicating if the tissue sample includes the skin condition.

9. The method of claim 8, wherein the skin condition includes at least one of cutaneous T cell lymphoma (CTCL), psoriasis, or atopic dermatitis.

10. The method of claim 8, wherein the gene data is obtained by isolating nucleic acids from the tissue sample, the tissue sample comprising cells from a stratum corneum.

11. The method of claim 10, wherein the nucleic acids comprise mRNA.

12. The method of claim 8, wherein the one or more diagnostic models is trained using historic gene data.

13. The method of claim 10, the tissue sample comprises cells from a subject having or suspected of having CTCL, eczema, psoriasis, atopic dermatitis, or contact dermatitis.

14. The method of claim 8, the gene data comprises expression level of one or more genes selected from a group consisting of FYB, LEF1, GNLY, DMN3, ITK, IL26, STAT5, TRAF3IP3, TNFSF11, CCL27, CXCL8, CXCL9, CXCL10, and TNF.

15. A system for detecting a skin disease, the system comprising:
a skin diagnostic system in communication with a computing device and one or more databases over a network, the skin diagnostic system receiving gene data, the gene data extracted from a tissue sample collected by a non-invasive skin sample collector;
one or more diagnostic models processing the gene data to generate prediction data associated with the skin disease; and
an output generation system generating output data indicating if the tissue sample includes the skin disease.

16. The system of claim 15, wherein the skin disease includes at least one of cutaneous T cell lymphoma (CTCL), psoriasis, or atopic dermatitis.

17. The system of claim 15, wherein the gene data is obtained by isolating nucleic acids from the tissue sample, the tissue sample comprising cells from a stratum corneum.

18. The system of claim 15, wherein the one or more diagnostic models is trained using historic gene data.

19. The system of claim 17, the tissue sample comprises cells from a subject having or suspected of having CTCL, eczema, psoriasis, atopic dermatitis, or contact dermatitis.

20. The system of claim 15, the gene data comprises expression level of one or more genes selected from a group consisting of FYB, LEF1, GNLY, DMN3, ITK, IL26, STAT5, TRAF3IP3, TNFSF11, CCL27, CXCL8, CXCL9, CXCL10, and TNF.
